(19) United States
(12) Patent Application Publication
(10) Pub. No.: US 2018/0186855 A1
(43) Pub. Date: Jul. 5, 2018

(54) CHIMERIC RECEPTORS AND METHODS OF USE THEREOF

(71) Applicant: ALECTOR LLC, South San Francisco, CA (US)

(72) Inventor: Arnon ROSENTHAL, Woodside, CA (US)

(73) Assignee: ALECTOR LLC, South San Francisco, CA (US)

(21) Appl. No.: 15/466,541

(22) Filed: Mar. 22, 2017

Related U.S. Application Data
(60) Provisional application No. 62/312,375, filed on Mar. 23, 2016.

(51) Int. Cl. C07K 14/725; A61K 35/14; A61K 49/00; C07K 14/715; C07K 16/18; C07K 14/705; A61K 39/00

(52) U.S. Cl. CPC C07K 14/7051; A61K 35/14; A61K 49/0004; C07K 14/7153; A61K 38/00; C07K 14/70517; A61K 39/0007; C07K 2319/02; C07K 2319/03; C07K 16/18

(57) ABSTRACT

The present disclosure is related to compositions that include polynucleotides encoding chimeric receptors, methods of delivering polynucleotides encoding chimeric receptors to immune cells, and methods of using immune cells encoding chimeric receptors to treat or prevent a neurological disease, disorder, or injury.

### CROSS-REFERENCE TO RELATED APPLICATIONS

This application claims the benefit of U.S. Provisional Application No. 62/312,375, filed Mar. 23, 2016, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022001100SEQLIST.TXT, date recorded: Mar. 21, 2017, size: 143 KB).

### FIELD OF THE INVENTION

The present disclosure relates to chimeric receptors and therapeutic uses of such chimeric receptors.

### BACKGROUND OF THE INVENTION

Human genome-wide association studies have demonstrated a key role for the innate immune system in neurological diseases (Wyss-Coray and Rogers, Cold Spring Harb Perspect Med, a006346, 2012, Heppner, Ransohoff et al., Nat Rev Neurosci, 358-372, 2015) (Gate, Rezai-Zadeh et al., J Neural Transm (Vienna), 961-970, 2010). The innate immune system is composed of myeloid cells such as monocytes, dendritic cells, neutrophils, circulating macrophages, and tissue macrophages including Kupfer cells, microglia, and Langerhans cells. Both resident myeloid cells in the CNS, such as microglia, and peripheral myeloid cells that traffic to the CNS, such as macrophages, are found in the context of pathology in Alzheimer's disease, frontotemporal dementia, and Parkinson's disease (Prinz, Priller et al., Nat Neurosci, 1227-1235, 2011; Meyer-Luehmann and Prinz, Trends Neurosci, 659-668, 2015).

Myeloid cells can have both protective and pathologic functions in the context of neurological diseases. Pathological processes include reduced repair functionality and a diminished ability to clear disease proteins (e.g. Abeta, Tau, and alpha synuclein). In addition, myeloid cells can contribute to the neuroinflammation that underlies many neurological diseases. Conversely, myeloid cells are capable of providing neuroprotective and anti-inflammatory effects, such as clearance of dead neurons and disease proteins and increased levels of growth factors.

Therefore, myeloid cells represent an attractive therapeutic target for neurological diseases, disorders, and injuries. For example, targeted modification of myeloid cell trafficking, activation, and function in the CNS may have a substantial positive impact on disease pathology. Accordingly, there is a need for approaches that modulate one or more myeloid cell activities, such as pro-repair functions, production of repair-associated cytokines, and clearance of pathological proteins in order to treat neurological diseases, disorders, and injuries.

All references cited herein, including patent applications and publications, are hereby incorporated by reference in their entirety.

### SUMMARY OF THE INVENTION

In order to meet the above needs, certain aspects of the present disclosure relate to a polynucleotide encoding a chimeric receptor, wherein the chimeric receptor comprises: (1) an extracellular ligand-binding domain, wherein the ligand is an agent associated with a neurological disease, disorder, or injury; (2) a transmembrane domain; and (3) a signaling domain, wherein binding of the ligand to the chimeric receptor expressed in an immune cell activates the signaling domain, and the activated signaling domain induces and/or enhances (i) cell survival of the immune cell, (ii) proliferation of the immune cell, (iii) migration of the immune cell, (iv) functionality of the immune cell, or any combination thereof. In some embodiments, the polynucleotide comprises a nucleic acid sequence selected from SEQ ID NOs: 38-53. Other aspects of the present disclosure relate to a polynucleotide comprising a nucleic acid sequence selected from SEQ ID NOs: 38-53.

In some embodiments that may be combined with any of the preceding embodiments, the chimeric receptor comprises an amino acid sequence selected form the group consisting of SEQ ID NOs: 22-37. In some embodiments that may be combined with any of the preceding embodiments, the ligand-binding domain is selected from a single-domain antibody, a nanobody, a heavy-chain antibody, a V_{NAR} fragment, a single-chain Fv domain (scFv), a V_{L} domain linked to a V_{H} domain by a flexible linker, an antibody Fab, and an extracellular domain of a receptor. In some embodiments that may be combined with any of the preceding embodiments, the agent associated with a neurological disease, disorder, or injury is selected from amyloid beta, Tau protein, and alpha-synuclein. In some embodiments that may be combined with any of the preceding embodiments, the agent associated with a neurological disease, disorder, or injury is selected from antisense GGCCCC (G2C4) repeat-expansion RNA, amyloid beta, oligomeric amyloid beta, amyloid beta plaques, amyloid precursor protein or fragments thereof, Tau protein, phosphorylated or truncated Tau protein, LAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides. In some embodiments that may be combined with any of the preceding embodiments, the ligand-binding domain is selected from an anti-amyloid beta single-chain variable fragment (scFv) domain, an anti-tau-NFT single-chain variable fragment (scFv) domain, and an anti-alpha-synuclein single-chain variable fragment (scFv) domain. In some embodiments that may be combined with any of the preceding embodiments, the neurological disease, disorder, or injury is selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, multiple sclerosis, and CNS herpes. In some embodiments that may be combined with any of the preceding embodiments, the transmembrane domain is a transmembrane domain from a protein selected from a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, a TLR (toll-like receptor), TLR5, Myd88, lymphocyte receptor chain, IL-2 receptor, IgE, IgG, CD16α, FcγRIII, FcγRII, CD28, 4-1BB, CD4, and CD8. In some embodiments that may be combined with any of the preceding embodiments, the transmembrane domain is a transmembrane domain selected from a CD8 transmembrane domain, a DAP12 transmembrane domain, a CASF-1R transmembrane domain, and a TLR5 transmembrane domain. In some embodiments that may be combined with any of the preceding embodiments, the signaling domain is a signaling domain are from one or more proteins selected from a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-KappaB, a TLR (toll-like receptor), TLR5, Myd88, TOR/CD3 complex, lymphocyte receptor chain, IL-2 receptor, IgE, IgG, CD16α, FcγRIII, FcγII, CD28, 4-1BB, and any combination thereof. In some embodiments that may be combined with any of the preceding embodiments, the signaling domain is a signaling domain selected from a 4-1BB intracellular domain, a CD3-zeta ITAM domain, a CD3-zeta intracellular domain, a CSF-1R receptor tyrosine kinase (RTK) intracellular domain, a DAP12 intracellular domain, a TCR-zeta intracellular domain, a TLR5 intracellular domain, a CD28 intracellular domain, a DAP10 intracellular domain, an FcR-gamma intracellular domain, and any combination thereof. In some embodiments that may be combined with any of the preceding embodiments, the immune cell is an innate immune cell. In some embodiments that may be combined with any of the preceding embodiments, the innate immune cell is a myeloid cell. In some embodiments that may be combined with any of the preceding embodiments, the innate immune cell is an innate immune cell selected from macrophages, M1 macrophages, activated M1 macrophages, M2 macrophages, neutrophils, NK cells, dendritic cells, monocytes, osteoclasts, Langerhans cells, Kupffer cells, microglia, M1 microglia, activated M1 microglia, M2 microglia, astrocytes, A1 astrocytes, A2 astrocytes, myeloid derived suppressor cells, and any combination thereof. In some embodiments that may be combined with any of the preceding embodiments, the immune cell is an adaptive immune cell. In some embodiments that may be combined with any of the preceding embodiments, the adaptive immune cell is an adaptive immune cell selected from T cells, T helper cells, cytotoxic T cells, memory T cells, regulatory T cells, natural killer T cells, mucosal associate invariant T cells, gamma delta T cells, B cells, memory B cells, follicular B cells, marginal zone B cells, B-1 cells, B-2 cells, regulatory B cells, and any combination thereof. In some embodiments that may be combined with any of the preceding embodiments, the chimeric receptor further comprises a flexible linker located between the transmembrane domain and the signaling domain. In some embodiments that may be combined with any of the preceding embodiments, the flexible linker is a flexible linker selected from a CD8 hinge domain, a TLR5 hinge domain, and a CSF-1R linker domain. In some embodiments that may be combined with any of the preceding embodiments, the chimeric receptor further comprises a signal peptide at the N-terminus of the chimeric receptor. In some embodiments that may be combined with any of the preceding embodiments, the signal peptide is a CD8 secretory signal peptide. In some embodiments that may be combined with any of the preceding embodiments, the chimeric receptor further comprises a heterodimerization domain. In some embodiments that may be combined with any of the preceding embodiments, the heterodimerization domain is an inducible heterodimerization domain. In some embodiments that may be combined with any of the preceding embodiments, the heterodimerization domain is a FK506 binding protein (FKBP) heterodimerization domain. In some embodiments that may be combined with any of the preceding embodiments, the heterodimerization domain is a T2089L mutant of FKBP-rapamycin binding domain (FRB*) heterodimerization domain. In some embodiments that may be combined with any of the preceding embodiments, binding of the ligand to the chimeric receptor expressed in the immune cell induces one or more activities selected from: a. TREM1 phosphorylation; b. DAP12 phosphorylation; c. activation of one or more tyrosine kinases; d. activation of phosphatidylinositol 3-kinase (PI3K); e. activation of protein kinase B; f. recruitment of phospholipase C-gamma (PLC-gamma) to a cellular plasma membrane, activation of PLC-gamma, or both; g. recruitment of TEC-family kinase dVav to a cellular plasma membrane; h. activation of nuclear factor-kB (NF-kB); i. inhibition of MAPK signaling; j. phosphorylation of linker for activation of T cells (LAT), linker for activation of B cells (LAB), or both; k. activation of IL-2-induced tyrosine kinase (Itk); l. modulation of one or more pro-inflammatory mediators selected from IFN-γ, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, MCP-1, and any combination thereof; m. modulation of one or more anti-inflammatory mediators selected from IL-4, IL-10, TGF-β, IL-13, IL-35, IL-16, IFN-α, IL-1Rα, VEGF, G-CSF, soluble receptors for TNF, soluble receptors for IL-6, and any combination thereof; n. phosphorylation of extracellular signal-regulated kinase (ERK); o. modulated expression of C-C chemokine receptor 7 (CCR7); p. induction of microglial cell chemotaxis toward CCL19 and CCL21 expressing cells; q. normalization of disrupted ITAM-dependent gene expression; r. recruitment of Syk, ZAP70, or both to an ITAM complex; s. increased activity of one or more ITAM-dependent genes or CSF-1R-dependent genes; t. increased maturation of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof; u. increased ability of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof to prime or modulate the function of T cells; v. enhanced ability, normalized ability, or both of bone marrow-derived dendritic cells to prime or modulate function of antigen-specific T cells; w. induction of osteoclast production, increased rate of osteoclastogenesis, or both; x. increased survival of dendritic cells, macrophages, M1 macrophages, activated M1 macrophages, M2 macrophages, monocytes, osteoclasts, Langerhans cells, Kupffer cells, microglia, M1 microglia, activated M1 microglia, M2 microglia, Astrocytes, A1 astrocytes, A2 astrocytes, or any combination thereof; y. increased function of dendritic cells, macrophages, M1 macrophages, activated M1 macrophages, M2 macrophages, microglia, M1 microglia, activated M1 microglia, M2 microglia, astrocytes, A1 astrocytes, A2 astrocytes, or any combination thereof; z. increasing phagocytosis by dendritic cells, macrophages, M1 macrophages, activated M1 macrophages, M2 macrophages, monocytes, microglia, M1 microglia, activated M1 microglia, M2 microglia, astrocytes, A1 astrocytes, A2 astrocytes, or any combination thereof; aa. induction of one or more types of clearance selected from apoptotic neuron clearance, nerve tissue debris clearance, non-nerve tissue debris clearance, bacteria clearance, other foreign body clearance, disease-causing protein clearance, disease-causing peptide clearance, disease-causing nucleic acid clearance, and any combination thereof; optionally wherein the disease-causing protein is selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA; bb. induction of phagocytosis of one or more of apoptotic neurons, nerve tissue debris, non-nerve tissue debris, dysfunctional synapses, bacteria, other foreign bodies, disease-causing proteins, disease-causing peptides, disease-causing nucleic acids, or any combination thereof; optionally wherein the disease-causing protein is selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA; cc. increased expression of one or more stimulatory molecules selected from CD83, CD86 MHC class II, CD40, and any combination thereof; dd. modulated expression of one or more proteins selected from C1qa, C1qB, C1qC, C1s, C1R, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, VEGF, PDL-1, PDL-2, ICOS, B7-H3, B7-H4, OX40L, FOXP3, IDO, CD39, CD73, CD80, CD86, CD83, CD11b, CD14, CD33, Siglec-5, Siglec-7, Siglec-9, IFN-gamma, IFN-alpha,IFN-beta, IL-18, IL-12, IL-10, IL-6, IL-2, IL-1 (beta and alpha), TNF-alpha, TGF-beta, IRF1, IRF3, STAT1, STAT3, HIF1-alpha, GMZA, GMZB, GZMH, PRF1, GNLY, CXCL9, CXCL10, CCL5, CX3CL1, CCL2, MADCAM1, ICAM1, VCAM1, VEGF, GMCSF, MCSF, S1c7a2, Cxc19, Serpinb2, Ptgs2, Cxc13, Cd38, Arg1, Mg12, Retn1a, Ear11, Tmem26, Mrc1, Socs2, Ch25h, Chi313, S1c17a2, Flt1, TIM3, LAG3, CD137, GAL9, OX40, GITR, Osteopontin, MID1, AXL, ITGAX, LPL, SPP1, ATP6VoD2, SIGLECH, CD33, TMEM119, EMR1, CDH23, GLO1, RASGRF2, and any combination thereof; ee. increased memory; and ff. reduced cognitive deficit. In some embodiments that may be combined with any of the preceding embodiments, the polynucleotide is a DNA polynucleotide. In some embodiments that may be combined with any of the preceding embodiments, the polynuzcleotide is an RNA polynucleotide.

Other aspects of the present disclosure relate to a vector comprising the polynucleotide of any of the preceding embodiments. In some embodiments, the vector is a lentiviral vector, a retroviral vector, a sleeping beauty vector, an AAV vector, or a non-viral plasmid vector.

Other aspects of the present disclosure relate to an isolated chimeric receptor encoded by the polynucleotide of any of the preceding embodiments.

In some embodiments that may be combined with any of the preceding embodiments, the ligand-binding domain binds a ligand associated with Alzheimer's disease pathology. In some embodiments that may be combined with any of the preceding embodiments, the ligand is Amyloid beta or tau. In some embodiments that may be combined with any of the preceding embodiments, the ligand-binding domain binds a ligand associated with Parkinson's disease pathology. In some embodiments that may be combined with any of the preceding embodiments, the ligand is alpha-synuclein. In some embodiments that may be combined with any of the preceding embodiments, the ligand-binding domain binds a ligand associated with amyotrophic lateral sclerosis pathology. In some embodiments that may be combined with any of the preceding embodiments, the ligand is a dipeptide repeat derived by RAN translation at the C9ORF72 gene.

Other aspects of the present disclosure relate to an isolated host cell comprising the polynucleotide of any of the preceding embodiments. Other aspects of the present disclosure relate to an isolated host cell comprising the vector of any of the preceding embodiments. Other aspects of the present disclosure relate to an isolated host cell comprising the chimeric receptor of any of the preceding embodiments.

In some embodiments that may be combined with any of the preceding embodiments, the isolated host cell is an immune cell. In some embodiments that may be combined with any of the preceding embodiments, the immune cell is an innate immune cell. In some embodiments that may be combined with any of the preceding embodiments, the innate immune cell is a myeloid cell. In some embodiments that may be combined with any of the preceding embodiments, the innate immune cell is selected from a macrophage, an M1 macrophage, an activated M1 macrophage, an M2 macrophage, a neutrophil, a NK cell, a dendritic cell, a monocyte, an osteoclast, a Langerhans cell, a Kupffer cell, a microglial cell, an M1 microglial cell, an activated M1 microglial cell, an M2 microglial cell, an astrocyte, an A1 astrocyte, and an A2 astrocyte. In some embodiments that may be combined with any of the preceding embodiments, the immune cell is an adaptive immune cell. In some embodiments that may be combined with any of the preceding embodiments, the adaptive immune cell is selected from a T cell, a T helper cell, a cytotoxic T cell, a memory T cell, a regulatory T cell, a natural killer T cell, a mucosal associate invariant T cell, a gamma delta T cell, a B cell, a memory B cell, a follicular B cell, a marginal zone B cell, a B-1 cell, a B-2 cell, and a regulatory B cell. In some embodiments that may be combined with any of the preceding embodiments, the host cell lacks one or more genes encoding one or more immune molecules that allow for recognition by the adaptive immune system. In some embodiments that may be combined with any of the preceding embodiments, the one or more immune molecules are MHC class I molecules, MHC class I co-receptors, MHC class II molecules, MHC class II co-receptors, or any combination thereof. In some embodiments that may be combined with any of the preceding embodiments, the one or more genes were deleted using a nuclease selected from a Cas9 nuclease, a TALEN, and a ZFN.

Other aspects of the present disclosure relate to an isolated myeloid cell expressing the chimeric receptor of any of the preceding embodiments, wherein the cell phenotype is modified in vitro or in vivo by addition of pro-inflammatory or anti-inflammatory agents or cytokines selected from GM-CSF, MCSF, IL-1, IL4, IL10, IL12, TNFα, TGF-beta, and LPS.

Other aspects of the present disclosure relate to an isolated myeloid cell comprising: (1) a first polynucleotide encoding: (i) a chimeric receptor, wherein the chimeric receptor comprises an extracellular ligand-binding domain, wherein the ligand is an agent associated with a neurological disease, disorder, or injury; (ii) a flexible linker; (iii) a transmembrane domain, and (iv) a heterodimerization domain; and (2) a second polynucleotide encoding: (i) a flexible linker, (ii) a transmembrane domain, (iii) a signaling domains, and (iv) a heterodimerization domain.

In some embodiments that may be combined with any of the preceding embodiments, the ligand-binding domain of the chimeric receptor is a single-chain Fv domain (scFv), the agent associated with a neurological disease, disorder, or injury of the chimeric receptor is amyloid beta, the flexible linker of the chimeric receptor is a CD8 hinge domain, the transmembrane domain of the chimeric receptor is a CD8 transmembrane domain, and the heterodimerization domain of the chimeric receptor is an inducible FK506 binding protein (FKBP) heterodimerization domain. In some embodiments that may be combined with any of the preceding embodiments, the flexible linker encoded by the second polynucleotide is a CSF-1R linker domain, the transmembrane domain encoded by the second polynucleotide is a CSF-1R 1 transmembrane domain, the one or more signaling domains encoded by the second polynucleotide are a CSF-1R receptor tyrosine kinase (RTK) intracellular domain and a CD3-zeta ITAM domain, and the heterodimerization domain encoded by the second polynucleotide is an inducible T2089L mutant of FKBP-rapamycin binding domain (FRB*) heterodimerization domain. In some embodiments that may be combined with any of the preceding embodiments, the first polynucleotide and the second polynucleotide each encode a polypeptide further comprising a CD8 secretory signal peptide at the N-terminus of the encoded polypeptide.

Other aspects of the present disclosure relate to a method of producing an immune cell expressing a chimeric receptor, comprising: (a) isolating an immune cell; (b) introducing the vector of any of the preceding embodiments into the cell; and (c) culturing the cell so that the chimeric receptor is expressed. In some embodiments, the immune cell is an innate immune cell. In some embodiments, the innate immune cell is a myeloid cell. In some embodiments, the innate immune cell is selected from a macrophage, an M1 macrophage, an activated M1 macrophage, an M2 macrophage, a neutrophil, a NK cell, a dendritic cell, a monocyte, an osteoclast, a Langerhans cell, a Kupffer cell, a microglial cell, an M1 microglial cell, an activated M1 microglial cell, an M2 microglial cell, an astrocyte, an A1 astrocyte, and an A2 astrocyte. In some embodiments, the immune cell is an adaptive immune cell. In some embodiments, the adaptive immune cell is selected from a T cell, a T helper cell, a cytotoxic T cell, a memory T cell, a regulatory T cell, a natural killer T cell, a mucosal associate invariant T cell, a gamma delta T cell, a B cell, a memory B cell, a follicular B cell, a marginal zone B cell, a B-1 cell, a B-2 cell, and a regulatory B cell.

Other aspects of the present disclosure relate to an isolated immune cell comprising a chimeric receptor produced by the method of any of the preceding embodiments.

In some embodiments that may be combined with any of the preceding embodiments, the cell further expresses one or more signaling factors that promote an M2 phenotype by inhibiting a TNF-alpha/NF-KappaB pathway a TLR/MyD88 pathway, or both. In some embodiments that may be combined with any of the preceding embodiments, the one or more signaling factors that promote an M2 phenotype by inhibiting a TNF-alpha/NF-KappaB pathway are one or more signaling factors selected from a dominant negative IKK-alpha, a dominant negative IKK-alpha IKK-beta, a dominant negative IKK-alpha IKBa (IKBa-DN), a MEKK isoform, and any combination thereof. In some embodiments that may be combined with any of the preceding embodiments, the one or more signaling factors that promote an M2 phenotype by inhibiting a TLR/MyD88 pathway are one or more dominant negative forms of MyD88.

Other aspects of the present disclosure relate to a pharmaceutical composition comprising the polynucleotide o of any of the preceding embodiments, and a pharmaceutically acceptable carrier. Other aspects of the present disclosure relate to a pharmaceutical composition comprising the vector of any of the preceding embodiments, and a pharmaceutically acceptable carrier. Other aspects of the present disclosure relate to a pharmaceutical composition comprising the chimeric receptor of any of the preceding embodiments, and a pharmaceutically acceptable carrier. Other aspects of the present disclosure relate to a pharmaceutical composition comprising the isolated cell of any of the preceding embodiments, and a pharmaceutically acceptable carrier.

Other aspects of the present disclosure relate to a method of preventing, reducing risk, or treating a neurological disease, disorder, or injury comprising administering to an individual in need thereof a therapeutically effective amount of the isolated cell of any of the preceding embodiments. Other aspects of the present disclosure relate to the isolated cell of any of the preceding embodiments for use in preventing, reducing risk, or treating a neurological disease, disorder, or injury in an individual in need thereof. Other aspects of the present disclosure relate to use of the isolated cell of any of the preceding embodiments in the manufacture of a medicament for preventing, reducing risk, or treating a neurological disease, disorder, or injury in an individual in need thereof.

Other aspects of the present disclosure relate to a method of preventing, reducing risk, or treating a neurological disease, disorder, or injury in an individual in need thereof, comprising: (a) obtaining a plurality of isolated immune cells; (b) introducing the vector of any of the preceding embodiments into the plurality of isolated immune cells; and (c) administering to the individual a therapeutically effective amount of the plurality of isolated immune cells containing the vector. Other aspects of the present disclosure relate to isolated immune cells containing the vector of any of the preceding embodiments for use in preventing, reducing risk, or treating a neurological disease, disorder, or injury in an individual in need thereof. Other aspects of the present disclosure relate to use of isolated immune cells containing the vector of any of the preceding embodiments in the manufacture of a medicament for preventing, reducing risk, or treating a neurological disease, disorder, or injury in an individual in need thereof.

In some embodiments that may be combined with any of the preceding embodiments, the plurality of isolated immune cells is administered peripherally into the individual without irradiation. In some embodiments that may be combined with any of the preceding embodiments, the vector contained in the plurality of isolated immune cells is expressed after administration of the plurality of immune cells to the individual. In some embodiments that may be combined with any of the preceding embodiments, the administered plurality of isolated immune cells infiltrates the central nervous system of the individual.

Other aspects of the present disclosure relate to a method for therapeutic delivery of cells to a central nervous system for preventing, reducing risk, or treating a neurological disease, disorder, or injury in an individual in need thereof, comprising: (a) obtaining a blood sample, a bone marrow sample, or a brain tissue sample from a suitable donor; (b) immunolabeling a plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells in the blood sample, the bone marrow sample, or the brain tissue sample; (c) isolating the immunolabeled plurality of cells from the blood sample, the bone marrow sample, or the brain tissue sample; (d) introducing the vector of any of the preceding embodiments into the isolated plurality of cells; and (e) administering to the periphery of the individual, without irradiation of the individual or any portion of the individual, a therapeutically effective amount of the plurality of cells containing the vector, wherein the administered plurality of cells infiltrates the central nervous system of the individual.

In some embodiments that may be combined with any of the preceding embodiments, the expression of the vector in the administered cells increases recruitment of the cells across the blood-brain barrier and into the central nervous system of the individual. In some embodiments that may be combined with any of the preceding embodiments, the administered cells further recruit myeloid cells having an M2-like protective phenotype or an A2-like protective phenotype into the central nervous system of the individual. In some embodiments that may be combined with any of the preceding embodiments, the cells induce production of endogenous neuronal survival agents, trophic agents, or both locally in the central nervous system of the individual. In some embodiments that may be combined with any of the preceding embodiments, binding of the ligand to the chimeric receptor induces an increase in myeloid cell activation, proliferation, survival, phagocytosis, and/or functionality. In some embodiments that may be combined with any of the preceding embodiments, the neurological disease, disorder, or injury is Alzheimer's disease and the cells infiltrate Amyloid beta plaques in the central nervous system of the individual. In some embodiments that may be combined with any of the preceding embodiments, the neurological disease, disorder, or injury is selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, multiple sclerosis, and CNS herpes. In some embodiments that may be combined with any of the preceding embodiments, the cells are one or more innate immune cells. In some embodiments that may be combined with any of the preceding embodiments, the one or more innate immune cells are myeloid cells. In some embodiments that may be combined with any of the preceding embodiments, the one or more innate immune cells are selected from macrophages, M1 macrophages, activated M1 macrophages, M2 macrophages, neutrophils, NK cells, dendritic cells, monocytes, osteoclasts, Langerhans cells, Kupffer cells, microglia, M1 microglia, activated M1 microglia, M2 microglia, astrocytes, A1 astrocytes, A2 astrocytes, and any combination thereof. In some embodiments that may be combined with any of the preceding embodiments, the cells are one or more adaptive immune cells. In some embodiments that may be combined with any of the preceding embodiments, the one or more adaptive immune cells are selected from T cells, T helper cells, cytotoxic T cells, memory T cells, regulatory T cells, natural killer T cells, mucosal associate invariant T cells, gamma delta T cells, B cells, memory B cells, follicular B cells, marginal zone B cells, B-1 cells, B-2 cells, regulatory B cells, and any combination thereof. In some embodiments that may be combined with any of the preceding embodiments, the administering induces one or more activities selected from: a. TREM1 phosphorylation; b. DAP12 phosphorylation; c. activation of one or more tyrosine kinases; d. activation of phosphatidylinositol 3-kinase (PI3K); e. activation of protein kinase B; f. recruitment of phospholipase C-gamma (PLC-gamma) to a cellular plasma membrane, activation of PLC-gamma, or both; g. recruitment of TEC-family kinase dVav to a cellular plasma membrane; h. activation of nuclear factor-kB (NF-kB); i. inhibition of MAPK signaling; j. phosphorylation of linker for activation of T cells (LAT), linker for activation of B cells (LAB), or both; k. activation of IL-2-induced tyrosine kinase (Itk); l. modulation of one or more pro-inflammatory mediators selected from IFN-γ, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, and MCP-1; m. modulation of one or more anti-inflammatory mediators selected from IL-4, IL-10, TGF-β, IL-13, IL-35, IL-16, IFN-α, IL-1Rα, VEGF, G-CSF, soluble receptors for TNF, and soluble receptors for IL-6; n. phosphorylation of extracellular signal-regulated kinase (ERK); o. modulated expression of C-C chemokine receptor 7 (CCR7); p. induction of microglial cell chemotaxis toward CCL19 and CCL21 expressing cells; q. normalization of disrupted ITAM-dependent gene expression; r. recruitment of Syk, ZAP70, or both to an ITAM complex; s. increased activity of one or more ITAM-dependent genes or CSF-1R-dependent genes; t. increased maturation of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof; u. increased ability of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof to prime or modulate the function of T cells; v. enhanced ability, normalized ability, or both of bone marrow-derived dendritic cells to prime or modulate function of antigen-specific T cells; w. induction of osteoclast production, increased rate of osteoclastogenesis, or both; x. increased survival of dendritic cells, macrophages, M1 macrophages, activated M1 macrophages, M2 macrophages, monocytes, osteoclasts, Langerhans cells, Kupffer cells, microglia, M1 microglia, activated M1 microglia, M2 microglia, Astrocytes, A1 astrocytes, A2 astrocytes, or any combination thereof; y. increased function of dendritic cells, macrophages, M1 macrophages, activated M1 macrophages, M2 macrophages, microglia, M1 microglia, activated M1 microglia, M2 microglia, astrocytes, A1 astrocytes, A2 astrocytes, or any combination thereof; z. increasing phagocytosis by dendritic cells, macrophages, M1 macrophages, activated M1 macrophages, M2 macrophages, monocytes, microglia, M1 microglia, activated M1 microglia, M2 microglia, astrocytes, A1 astrocytes, A2 astrocytes, or any combination thereof; aa. induction of one or more types of clearance selected from apoptotic neuron clearance, nerve tissue debris clearance, non-nerve tissue debris clearance, bacteria clearance, other foreign body clearance, disease-causing protein clearance, disease-causing peptide clearance, disease-causing nucleic acid clearance, and any combination thereof; optionally wherein the disease-causing protein is selected from amyloid beta, oligomeric amyloid beta, amyloid beta plaques, amyloid precursor protein or fragments thereof, Tau, LAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA; bb. induction of phagocytosis of one or more of apoptotic neurons, nerve tissue debris, non-nerve tissue debris, dysfunctional synapses, bacteria, other foreign bodies, disease-causing proteins, disease-causing peptides, disease-causing nucleic acids, or any combination thereof; optionally wherein the disease-causing protein is selected from amyloid beta, oligomeric amyloid beta, amyloid beta plaques, amyloid precursor protein or fragments thereof, Tau, LAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA; cc. increased expression of one or more stimulatory molecules selected from CD83, CD86 MHC class II, CD40, and any combination thereof; dd. modulated expression of one or more proteins selected from C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOX5AP, ITGAM, SLC7A7, CD4, ITGAX, PYCARD, VEGF, PDL-1, PDL-2, ICOS, B7-H3, B7-H4, OX40L, FOXP3, IDO, CD39, CD73, CD80, CD86, CD83, CD11b, CD14, CD33, Siglec-5, Siglec-7, Siglec-9, IFN-gamma, IFN-alpha,IFN-beta, IL-18, IL-12, IL-10, IL-6, IL-2, IL-1 (beta and alpha), TNF-alpha, TGF-beta, IRF1, IRF3, STAT1, STAT3, HIF1-alpha, GMZA, GMZB, GZMH, PRF1, GNLY, CXCL9, CXCL10, CCL5, CX3CL1, CCL2, MADCAM1, ICAM1, VCAM1, VEGF, GMCSF, MCSF, S1c7a2, Cxc19, Serpinb2, Ptgs2, Cxc13, Cd38, Arg1, Mg12, Retn1a, Ear11, Tmem26, Mrc1, Socs2, Ch25h, Chi313, S1c17a2, Flt1, TIM3, LAG3, CD137, GAL9, OX40, GITR, Osteopontin, MID1, AXL, ITGAX, LPL, SPP1, ATP6VoD2, SIGLECH, CD33, TMEM119, EMR1, CDH23, GLO1, RASGRF2, and any combination thereof; ee. increased memory; and ff. reduced cognitive deficit.

Other aspects of the present disclosure relate to a method of testing efficacy of therapeutic delivery of cells to a central nervous system for preventing, reducing risk, or treating a neurological disease, disorder, or injury in an individual in need thereof, comprising: obtaining a blood sample or a bone marrow sample from a suitable donor; immunolabeling a plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells in the blood sample or the bone marrow sample; isolating the immunolabeled plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells from the blood sample or the bone marrow sample; introducing the vector of any of the preceding embodiments into the isolated plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells; administering to the periphery or brain of the individual, without irradiation of the individual or any portion of the individual, a therapeutically effective amount of the plurality of cells containing the vector, wherein the administered plurality of cells infiltrates the locus of the neurological disease, disorder, or injury within the central nervous system of the individual; detecting the presence of the administered plurality of cells at the locus of the neurological disease, disorder, or injury within the central nervous system of the individual; and determining the therapeutic effect of the administered plurality of cells at the locus of the neurological disease, disorder, or injury.

### BRIEF DESCRIPTION OF THE DRAWINGS

FIG. 1A and FIG. 1B show a schematic of the SMART2 chimeric receptor structure (FIG. 1A) and a schematic of a vector that harbors this receptor cloned into pCDNA3.4-Topo from Life Technologies (FIG. 1B). SMART2 is composed of the elements: CD8 secretory signal sequence (SS)>>antiAbeta scFv>>CD8 Hinge domain>>CD8 transmembrane domain (TM)>>CD3Zeta ITAM domain.

FIG. 2A and FIG. 2B show a schematic of the SMART3 chimeric receptor structure (FIG. 2A) and a schematic of a vector that harbors this receptor cloned into pCDNA3.4-Topo from Life Technologies (FIG. 2B). SMART3 is composed of the elements: CD8SS>>anti-Abeta scFv>>CD8 Hinge>>CD8TM>>CSF1R receptor tyrosine kinase (RTK) Intracellular domain.

FIG. 3A and FIG. 3B show a schematic of the SMART4 chimeric receptor structure (FIG. 3A) and a schematic of a vector that harbors this receptor cloned into pCDNA3.4-Topo from Life Technologies (FIG. 3B). SMART4 is composed of the elements CD8 SS>>anti-Abeta scFv>>CD8 Hinge>>CD8TM>>CSF1R RTK Intracellular domain>>CD3Zeta ITAM domain. The sequences for SMART4 are sequence24 and sequence25.

FIG. 4A and FIG. 4B show a schematic of the SMART5 chimeric receptor structure (FIG. 4A) and a schematic of a vector that harbors this receptor cloned into pCDNA3.4-Topo from Life Technologies (FIG. 4B). SMART5 is composed of the elements: CD8 SS>>anti-Abeta scFv>>CD8 Hinge>>DAP12 transmembrane and full-length Intracellular domains.

FIG. 5A and FIG. 5B show a schematic of the SMART6 chimeric receptor structure (FIG. 5A) and a schematic of a vector that harbors this receptor cloned into pCDNA3.4-Topo from Life Technologies (FIG. 5B). SMART6 is composed of the elements: CD8 SS>>svFv anti-Tau_NFT>>CD8 Hinge>>DAP12 transmembrane and full-length intracellular domain.

FIG. 6A and FIG. 6B show a schematic of the SMART7 chimeric receptor structure (FIG. 6A) and a schematic of a vector that harbors this receptor cloned into pCDNA3.4-Topo from Life Technologies (FIG. 6B). SMART7 is composed of the elements CD8SS>>anti-Tau_NFT scFv>>CD8 Hinge>>CD8 transmembrane>TCRzeta intracellular domain.

FIG. 7A and FIG. 7B show a schematic of the SMART8 chimeric receptor structure (FIG. 7A) and a schematic of a vector that harbors this receptor cloned into pCDNA3.4-Topo from Life Technologies (FIG. 7B). SMART8 is composed of the elements: CD8SS>>anti-Tau_NFT scFv>>CD8 Hinge>>CD8 transmembrane>CSF1R RTK intracellular domain.

FIG. 8A and FIG. 8B show a schematic of the SMART9 chimeric receptor structure (FIG. 8A) and a schematic of a vector that harbors this receptor cloned into pCDNA3.4-Topo from Life Technologies (FIG. 8B). SMART9 is composed of the elements CD8 SS>>anti-Abeta scFv>>CSF1R linker, Transmembrane and RTK Intracellular domains.

FIG. 9A and FIG. 9B show a schematic of the SMART10 chimeric receptor structure (FIG. 9A) and a schematic of a vector that harbors this receptor cloned into pCDNA3.4-Topo from Life Technologies (FIG. 9B). SMART10 is composed of the elements CD8 SS>>anti-Abeta scFv>>CSF1R Linker, Transmembrane, and RTK Intracellular domain>>CD3Zeta ITAM domain.

FIG. 10A and FIG. 10B show a schematic of the SMART17 chimeric receptor structure (FIG. 10A) and a schematic of a vector that harbors this receptor cloned into pCDNA3.4-Topo from Life Technologies (FIG. 10B). SMART17 is composed of the elements CD8SS>>anti-alpha-Synuclein scFv>>CD8Hinge>>CD8TM>>CD3Zeta ITAM domain.

FIG. 11A and FIG. 11B show a schematic of the SMART18 chimeric receptor structure (FIG. 11A) and a schematic of a vector that harbors this receptor cloned into pCDNA3.4-Topo from Life Technologies (FIG. 11B). SMART18 is composed of the elements CD8SS>>anti-alpha-Synuclein scFv>>CD8Hinge>>CD8TM>>CD3Zeta ITAM domain.

FIG. 12A and FIG. 12B show a schematic of the SMART19 chimeric receptor structure (FIG. 12A) and a schematic of a vector that harbors this receptor cloned into pCDNA3.4-Topo from Life Technologies (FIG. 12B). SMART19 is composed of the elements CD8 SS>>anti-AlphaSynuclein>>CD8 Hinge>>DAP12 transmembrane and full-length Intracellular domains.

FIG. 13A and FIG. 13B show a schematic of the SMART20A chimeric receptor component structure (FIG. 13A) and a schematic of a vector that harbors this receptor component cloned into pCDNA3.4-Topo from Life Technologies (FIG. 13B). SMART20A is composed of the elements CD8 SS>>anti-Abeta scFv>>the CD8 Hinge>>CD8 transmembrane domain>>4-1BB signaling domain ahead of the FKBP inducible dimerization domain. SMART20A is part of the two-component inducible SMART20 receptor, which includes SMART20B.

FIG. 14A and FIG. 14B show a schematic of the SMART20B chimeric receptor component structure (FIG. 14A) and a schematic of a vector that harbors this receptor component cloned into pCDNA3.4-Topo from Life Technologies (FIG. 14B). SMART20B is composed of the elements CD8SS>>CD8 Hinge>>DAP12 full length>>FRB+ dimerization domain. SMART20B is part of the two-component inducible SMART20 receptor, which includes SMART20A.

FIG. 15A and FIG. 15B show a schematic of the SMART21A chimeric receptor component structure (FIG. 15A) and a schematic of a vector that harbors this receptor component cloned into pCDNA3.4-Topo from Life Technologies (FIG. 15B). SMART21A is composed of the elements CD8SS>>anti-Abeta>>CD8 Hinge>>CD8TM>>FKBP. SMART21A is part of the two-component inducible SMART21 receptor, which includes SMART21B.

FIG. 16A and FIG. 16B show a schematic of the SMART21B chimeric receptor component structure (FIG. 16A) and a schematic of a vector that harbors this receptor component cloned into pCDNA3.4-Topo from Life Technologies (FIG. 16B). SMART21B is composed of the elements CD8SS>>CSF1R Linker, Transmembrane, and RTK Intracellular domain>>FRB+>>CD3Zeta ITAM domain. SMART21B is part of the two-component inducible SMART21 receptor, which includes SMART21A.

### DETAILED DESCRIPTION OF THE INVENTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Definitions

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to effect a measurable improvement of a particular disease, disorder, or condition. A therapeutically effective amount herein may vary according to factors such as the disease state, age, sex, and weight of the patient, and the ability of the chimeric receptors to elicit a desired response in the individual. A therapeutically effective amount is also one in which any toxic or detrimental effects of the chimeric receptors are outweighed by the therapeutically beneficial effects.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment includes an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment includes of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

“Functional fragments” of antibodies, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)).

As use herein, the term “specifically recognizes” or “specifically binds” refers to measurable and reproducible interactions such as attraction or binding between a ligand and a chimeric receptor that is determinative of the presence of the target in the presence of a heterogeneous population of molecules including biological molecules. For example, a chimeric receptor of the present disclosure, that specifically or preferentially binds to a ligand or target with greater affinity, avidity, more readily, and/or with greater duration than it binds to other ligands or targets. It is also understood by reading this definition that, for example, a chimeric receptor that specifically or preferentially binds to a first target may or may not specifically or preferentially bind to a second target. As such, “specific binding” or “preferential binding” does not necessarily require (although it can include) exclusive binding. An chimeric receptor that specifically binds to a target may have an association constant of at least about 10³M⁻¹ or 10⁴M⁻¹, sometimes about 10⁵M⁻¹ or 10⁶M⁻¹, in other instances about 10⁶M⁻¹ or 10⁷M⁻¹, about 10⁸M⁻¹ to 10⁹M⁻¹, or about 10¹⁰M⁻¹ to 10¹¹M⁻¹ or higher. A variety of immunoassay formats can be used to select chimeric receptors specifically immunoreactive with a particular protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

The term “isolated” refers a molecule or cell that is identified and separated from at least one contaminant molecule or cell with which it is ordinarily associated in the environment in which it was produced. Preferably, the isolated molecule or cell is free of association with all components associated with the production environment. The isolated molecule or cell is in a form other than in the form or setting in which it is found in nature.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vitro, in vivo, or ex vivo with a polynucleotide(s) of the present disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

The term “ligand” as used herein refers to a molecule that binds to another molecule, such as a receptor or an antibody. For example, as used herein, a ligand is any compound or agent bound by a chimeric receptor's ligand-binding domain. Exemplary ligands include nucleic acids, peptides, or proteins associated with a neurological disease, disorder, or injury.

The term “nanobody,” also called a single-domain antibody, as used herein refers to an antibody fragment that includes a single monomeric variable antibody domain that binds to a specific antigen. Nanobodies may include a peptide chain of about 110 amino acids and may have one variable domain of a heavy-chain antibody or of a common IgG.

The term “V_{NAR}” as used herein refers to a single variable new antigen receptor (NAR) domain antibody fragment. V_{NAR} fragments are single-domain antibody fragments derived from heavy-chain antibodies, such as shark immunoglobulin new antigen receptor antibodies (IgNARs).

The term “extracellular receptor domain” as used herein refers to the portion of a cell bound receptor protein that is found externally on a cell. The extracellular receptor domain functions by binding to a ligand. For example, nucleic acids, peptides, proteins, or atomic ions may each bind to an extracellular receptor domain as a ligand.

The term “autologous” refers to any material derived from the same individual to whom it is later to be re-introduced.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the disclosures described herein include “comprising,” “consisting,” and “consisting essentially of” aspects and embodiments.

### Overview

The present disclosure relates to chimeric receptors comprising an extracellular ligand-binding domain that binds an agent associated with a neurological disease, disorder, or injury; a transmembrane domain; and a signaling domain. The present disclosure includes polynucleotides encoding chimeric receptors, and vectors comprising said polynucleotides. The present disclosure also includes immune cells expressing said chimeric receptors and methods of producing such immune cells by introducing polynucleotides or vectors encoding chimeric receptors into the cells. In some embodiments, immune cells expressing chimeric receptors of the present disclosure are administered to an individual to treat or prevent a neurological disease, disorder, or injury. In some embodiments, binding of the ligand to the chimeric receptor expressed in an immune cell activates the signaling domain, and the activated signaling domain induces and/or enhances cell survival of the immune cell, proliferation of the immune cell, migration of the immune cell, and/or functionality of the immune cell.

In some embodiments, the chimeric receptors expressed in myeloid cells of the present disclosure can be used to promote the survival of myeloid cells, to polarize these cells towards a protective and therapeutic phenotype, to promote their accumulation in the CNS at sites of disease pathology, and to enhance their activity against pathologies associated with neurological diseases, disorders, or injuries.

### Chimeric Receptors

Certain aspects of the present disclosure relate to a chimeric receptor. A chimeric receptor, as used herein, refers to a set of polypeptides, which when in an immune cell, provides the cell with specificity for a target ligand and with intracellular signal generation. In some aspects, the set of polypeptides are contiguous with each other, e.g., are in the same polypeptide chain (e.g., comprise a chimeric fusion protein). In some embodiments, the set of polypeptides are not contiguous with each other, e.g., are in different polypeptide chains. A chimeric receptor described herein at least comprises an extracellular ligand-binding domain, a transmembrane domain, and a cytoplasmic signaling domain (also referred to herein as “an intracellular signaling domain”) comprising a functional signaling domain derived from a stimulatory molecule and/or costimulatory molecule. In one aspect, the cytoplasmic signaling domain further comprises one or more functional signaling domains derived from at least one costimulatory molecule. In some embodiments, the extracellular domain of the chimeric receptor binds a ligand and transmits a signal to the cytoplasmic domain which transduces an effector function signal to the cell in which the receptor is expressed.

In some embodiments, the chimeric receptor includes two proteins, with each protein including one or more domains. For example, a chimeric receptor of the present disclosure can be a two-component receptor. Two-component chimeric receptors include two separate polypeptides that can associate, dimerize, or multimerize through an interaction domain. In some embodiments the chimeric receptor further comprises a flexible linker located between the transmembrane domain and the signaling domain. The flexible linker allows the ligand-binding domain to orient in different directions to facilitate ligand recognition and binding. Exemplary flexible linkers include, without limitation, a CD8 hinge domain, a TLR5 hinge domain, and a CSF-1R linker domain. In some embodiments, the chimeric receptor further comprises a signal peptide at the N-terminus of the chimeric receptor. The signal peptide directs the nascent chimeric receptor protein into the endoplasmic reticulum. This allows the receptor to be glycosylated and anchored in the cell membrane. In some embodiments, the signal peptide is a CD8 secretory signal peptide.

Ligand-Binding Domains

In some embodiments, chimeric receptors of the present disclosure include a ligand-binding domain. A ligand-binding domain refers to any suitable protein which binds to a specific ligand. The binding domain may include a part of antibody that binds to an antigen, such as an immunoglobulin chain or fragment comprising at least one immunoglobulin variable domain sequence. The portion of the chimeric receptor that includes an antibody or antibody fragment may exist in a variety of forms where the antigen binding domain is expressed as part of a contiguous polypeptide chain. The ligand-binding domain can be any domain that binds to a ligand including but not limited to a monoclonal antibody, a polyclonal antibody, a recombinant antibody, a murine antibody, a human antibody, a humanized antibody, and a functional fragment thereof, including but not limited to a single-domain antibody such as a heavy chain variable domain (VH), a light chain variable domain (VL), a variable domain (VHH) of a camelid derived nanobody, a heavy-chain antibody, a single domain antibody fragment (V_{NAR}) fragment, a single-chain Fv domain (scFv), a V_{L} domain linked to a V_{H} domain by a flexible linker, or an antibody Fab. In some embodiments, the ligand-binding domain is a scFv. ScFv molecules can be produced by linking VH and VL regions together using flexible polypeptide linkers. In some embodiments, scFv molecules comprise a linker (e.g., a Ser-Gly linker) with an optimized length and/or amino acid composition. The linker length can greatly affect how the variable regions of a scFv fold and interact. The linker sequence may comprise any naturally occurring amino acid. In some embodiments, the linker sequence comprises amino acids glycine and serine.

In some instances, it is beneficial for the ligand-binding domain to be derived from the same species in which the chimeric receptor will ultimately be used in. For example, for use in humans, it may be beneficial for the ligand-binding domain of the chimeric receptor to comprise human or humanized residues for the ligand-binding domain of an antibody or antibody fragment.

The ligand-binding domain may alternatively include a ligand-binding portion of a cell receptor protein. For example, the ligand-binding portion can include an extracellular receptor domain. An extracellular receptor domain includes the portion of a cell bound receptor protein that is found externally on the cell. Exemplary extracellular receptor domains, include, without limitation, those derived from a TCRs, MHC molecules, TNF receptor proteins, Immunoglobulin-like proteins, cytokine receptors, integrins, signaling lymphocytic activation molecules (SLAM proteins), and activating NK cell receptors. Examples of such molecules include, TREM1, TREM2, CD33, Siglec-5, 5, Siglec-7, Siglec-9, Siglec-11, CD27, CD28, 4-1BB (CD137), OX40, GITR, CD30, CD40, ICOS, BAFFR, HVEM, ICAM-1, lymphocyte function-associated antigen-1 (LFA-1), CD2, CD8, CD7, CD287, NKG2C, NKG2D, SLAMF7, NKp80, NKp30, NKp44, NKp46, CD160, B7-H3. and a ligand that specifically binds with CD83.

The ligand-binding domain of the present disclosure may bind any suitable ligand. Exemplary ligands include, without limitation, peptidies, proteins, sialic acids, lipids, lipoprotein and nucleic acids. The choice of extracellular ligand-binding domain depends upon the type and number of ligands that define the target of the chimeric receptor. In some embodiments, the ligand-binding domain may be chosen to recognize an agent that is associated with a disease state. In some embodiments, the ligand-binding domain may bind an agent associated with a neurological diseases, disorder, or injury. Exemplary neurological diseases, disorders, or injuries include, without limitation, dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, multiple sclerosis, and CNS herpes.

In some embodiments, the chimeric receptor-mediated immune cell response can be directed to an agent of interest by way of engineering a ligand-binding domain that specifically binds a desired agent into the chimeric receptor. The ligand-binding domain can be designed to specifically target an agent associated with a neurological disease, disorder, or injury. In some embodiments, the agent is a nucleic acid, peptide, lipid, lipoprotein, sialic acid, or protein associated with a neurological disease, disorder, or injury. In some embodiments, the agent is a wild-type nucleic acid, peptide, or protein. In some embodiments, the agent is a mutant nucleic acid, peptide, or protein. Exemplary agents associated with neurological diseases, disorders, or injuries include, without limitation, amyloid beta, Tau protein, and alpha-synuclein, antisense GGCCCC (G2C4) repeat-expansion RNA, oligomeric amyloid beta, amyloid beta plaques, amyloid precursor protein or fragments thereof, phosphorylated or truncated Tau protein, Islet amyloid polypeptide (IAPP), transactive response DNA binding protein 43 (TDP-43), Fused in Sarcoma (FUS) protein, chromosome 9 open reading frame 72 (C9orf72), chromosome 9 Repeat-associated non-ATG (c9RAN) protein, prion protein, prior protein scrapie associated (PrPSc), huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein A1, serum amyloid A, medin, prolactin, transthyretin, lysozyme, beta 2 microglobulin, gelsolin, keratoepithelin, cystatin, immunoglobulin light chain AL, sporadic inclusion body myosititis (S-IBM) protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides.

In some embodiments, the ligand-binding domain binds a ligand associated with Alzheimer's disease pathology. Exemplary ligands associated with Alzheimer's disease include amyloid beta protein or tau protein. Amyloid beta is a peptide 36-43 amino acids in length, which is formed by cleavage of amyloid precursor protein (APP) by beta secretase and gamma secretase. Amyloid beta molecules can aggregate to form oligomers and are the main components of amyloid plaques found during Alzheimer's disease. Tau protein is a highly soluble microtubule-associated protein (MAP). Abnormal phosphorylation of tau can result in the formation of tau neurofibrillary tangles (tau-NFTs) in the CNS. In some embodiments, the ligand is a wild-type amyloid beta protein or wild-type tau protein. In some embodiments, the ligand is a mutant amyloid beta protein or mutant tau protein. In some embodiments, the ligand is a plaque, aggregate or protein multimer comprising Abeta or Tau protein. In some embodiments, the ligand-binding domain binds a ligand associated with Parkinson's disease pathology, such as alpha-synuclein. In some embodiments, the ligand is wild-type alpha-synuclein protein. In some embodiments, the ligand is a mutant alpha-synuclein protein. In some embodiments, the ligand is a plaque, aggregate or protein multimer comprising alpha-synucleic protein. In some embodiments, the ligand-binding domain binds a ligand associated with amyotrophic lateral sclerosis pathology. For example, in some embodiments, the ligand is a dipeptide repeat derived by RAN translation at the C9ORF72 gene.

In some embodiments, the ligand-binding domain includes a single-chain variable fragment (scFv) domain that binds to a specific disease agent. Exemplary scFV domains include, without limitation, an anti-amyloid beta scFv domain, an anti-tau-NFT scFv domain, and an anti-alpha-synuclein scFv domain, an anti-antisense GGCCCC (G2C4) repeat-expansion RNA scFV domain, an anti-Islet amyloid polypeptide (IAPP) scFV domain, an anti-transactive response DNA binding protein 43 (TDP-43) scFV domain, an anti-Fused in Sarcoma (FUS) protein scFV domain, an anti-chromosome 9 open reading frame 72 (C9orf72) protein scFV domain, an anti-chromosome 9 Repeat-associated non-ATG (c9RAN) protein scFV domain, an anti-prion protein scFV domain, an anti-prion protein scrapie associated (PrPSc) scFV domain, an anti-huntingtin scFV domain, an anti-calcitonin scFV domain, an anti-superoxide dismutase scFV domain, an anti-ataxin scFV domain, an anti ataxin-1 scFV domain, an anti ataxin-2 scFV domain, an anti a an anti ataxin-3 scFV domain, an anti ataxin-7 scFV domain, an anti ataxin-8 scFV domain, taxin-10 scFV domain, an anti-Lewy body scFV domain, an anti-atrial natriuretic factor scFV domain, an anti-apolipoprotein A1 scFV domain, an anti-serum amyloid A scFV domain, an anti-lysozyme scFV domain, an anti-beta 2 microglobulin scFV domain, an anti-gelsolin scFV domain, an anti-keratoepithelin scFV domain, an anti-cystatin scFV domain, an anti-immunoglobulin light chain AL scFV domain, an anti-sporadic inclusion body myosititis (S-IBM) protein scFV domain, an anti-Repeat-associated non-ATG (RAN) translation product scFV domain, an anti-DiPeptide repeat (DPR) peptide scFV domain, an anti-glycine-alanine (GA) repeat peptide scFV domain, an anti-glycine-proline (GP) repeat peptide scFV domain, an anti-glycine-arginine (GR) repeat peptide scFV domain, an anti-proline-alanine (PA) repeat peptide scFV domain, an anti-ubiquitin scFV domain, an-anti proline-arginine (PR) repeat peptide scFV domain, and an anti-prion protein (PrP) scFV domain. In some embodiments, the anti-amyloid beta scFv domain binds an amyloid beta protein, plaque, aggregate, or protein multimer comprising amyloid beta protein. In some embodiments, the anti-tau-NFT scFV domain binds a tau protein, plaque, aggregate, or protein multimer comprising tau-NFT protein. In some embodiments, the anti-alpha-synuclein scFV domain binds an alpha-synuclein protein, plaque, aggregate, or protein multimer comprising alpha-synuclein protein. In some embodiments, the anti-antisense GGCCCC (G2C4) repeat-expansion RNA scFV domain binds GGCCCC (G2C4) repeat-expansion RNA. In some embodiments, the anti-IAPP scFV domain binds IAPP. In some embodiments, the anti-TDP-43 scFV domain bindsTDP-43 or TDP-43 peptides. In some embodiments, the ant-FUS protein scFV domain binds FUS. In some embodiments, the anti-C9orf72 protein scFV domain binds C9orf72. In some embodiments, the anti-c9RAN protein scFV domain binds c9RAN. In some embodiments, the anti-prion protein scFV domain binds prion protein. In some embodiments, the anti-PrPSc scFV domain binds PrPSc. In some embodiments the anti-huntingtin scFV domain binds huntingtin. In some embodiments the anti-calcitonin scFV domain binds calcitonin. In some embodiments the anti-superoxide dismutase scFV domain binds superoxide dismutase. In some embodiments the anti-ataxin scFV domain binds ataxin. In some embodiments the anti ataxin-1 scFV domain binds ataxin-1. In some embodiments the anti-ataxin-2 scFV domain binds ataxin-2. In some embodiments the anti-ataxin-3 scFV domain binds ataxin-3. In some embodiments the anti-ataxin-7 scFV domain binds ataxin-7. In some embodiments the anti-ataxin-8 scFV domain binds ataxin-8. In some embodiments the anti-ataxin-10 scFV domain binds ataxin-10. In some embodiments the anti-Lewy body scFV domain binds Lewy body. In some embodiments the anti-atrial natriuretic factor scFV domain binds atrial natriuretic factor. In some embodiments the anti-apolipoprotein A1 scFV domain binds apolipoprotein A1. In some embodiments the anti-serum amyloid A scFV domain binds serum amyloid A. In some embodiments the anti-lysozyme scFV domain binds lysozyme. In some embodiments the anti-beta 2 microglobulin scFV domain binds beta 2 microglobulin. In some embodiments the anti-gelsolin scFV domain binds gelsolin. In some embodiments the anti-keratoepithelin scFV domain binds keratoepithelin. In some embodiments the anti-cystatin scFV domain binds cystatin. In some embodiments the anti-immunoglobulin light chain AL scFV domain binds immunoglobulin light chain AL. In some embodiments the anti-S-IBM protein scFV domain binds S-IBM. In some embodiments the anti-RAN translation products scFV domain binds Repeat-associated non-ATG (RAN) translation product. In some embodiments the anti-DPR peptide scFV domain binds DiPeptide repeat (DPR) peptides. In some embodiments the anti-glycine-alanine (GA) repeat peptide scFV domain binds glycine-alanine (GA) repeat peptides. In some embodiments the anti-glycine-proline (GP) repeat peptide scFV domain binds glycine-proline (GP) repeat peptides. In some embodiments the anti-glycine-arginine (GR) repeat peptide scFV domain binds glycine-arginine (GR) repeat peptides. In some embodiments the anti-proline-alanine (PA) repeat peptide scFV domain binds proline-alanine (PA) repeat peptides. In some embodiments the anti-ubiquitin scFV domain binds ubiquitin. In some embodiments the anti-proline-arginine (PR) repeat peptide scFV domain binds proline-arginine (PR) repeat peptides. In some embodiments the anti-PrP scFV domain binds prion protein.

Transmembrane Domains

In some embodiments, chimeric receptors of the present disclosure comprise a transmembrane domain. As used herein, a transmembrane domain refers to a portion of a protein structure that is located in a membrane. Transmembrane domains may be a single alpha helix, a transmembrane beta barrel, or any other structure which is thermodynamically stable in a membrane. The transmembrane domain of the chimeric receptor may be derived from any membrane bound or transmembrane protein.

In some embodiments, the chimeric receptor may be designed to include a transmembrane domain that is fused to the extracellular ligand-binding domain of the chimeric receptor. In one embodiment, the transmembrane domain that naturally is associated with one of the domains in the chimeric receptor is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived from a natural source. For example, the domain may be derived from any membrane bound or transmembrane protein. Transmembrane regions for use in the chimeric receptors disclosed herein may be derived from a protein including, without limitation, a receptor tyrosine kinase (RTK), an macrophage colony-stimulating factor (M-CSF) receptor, colony stimulating factor 1 receptor (CSF-1R), Kit, Tetrahymena insertion-homing endonuclease 3 (TIE3), an immunoreceptor tyrosine-based activation motif (ITAM)-containing protein, DNAX-activation protein 12 (DAP12), DNAX-activation protein 10 (DAP10), an Fc receptor, FcR-gamma, FcR-epsilon, FcR-beta, T cell receptor zeta (TCR-zeta), cluster of differentiation (CD) 3-gamma, CD3-delta, CD3-epsilon, CD3-zeta, CD3-eta, CD5, CD22, CD79a, CD79b, CD66d, tumor necrosis factor (TNF)-alpha, nuclear factor kappa-light-chain-enhancer of activated B cells (NF-kappaB), a toll-like receptor (TLR), TLR5, myeloid differentiation primary response gene 88 (Myd88), lymphocyte receptor chain, interleukin-2 (IL-2) receptor, Immunoglobulin E (IgE), Immunoglobulin G (IgG), CD16α, FcγRIII, FcγRII, CD28, 4-1BB, CD4, and CD8. In some embodiments, the transmembrane domain is a CD8 transmembrane domain, a DAP12 transmembrane domain, a cCSF-1R transmembrane domain, or a TLR5 transmembrane domain.

Signaling Domains

In some embodiments, chimeric receptors of the present disclosure comprise a signaling domain. As used herein, a signaling domain refers to the functional portion of a protein which acts by transmitting information within the cell to regulate cellular activity via defined signaling pathways by generating second messengers or functioning as effectors by responding to such messengers.

In some embodiments, a signaling domain of the present disclosure may refer to the portion of a chimeric receptor which transduces the effector function signal, resulting in functional activities of the immune cell in which the chimeric receptor has been placed. Functional activities of an immune cell, for example, may be phagocytosis, secretion of cytokines, or trafficking. In some embodiments, the signaling domains promote function, migration, survival, and proliferation of immune cells. The entire intracellular signaling domain can be employed, or a truncated portion of the intracellular signaling domain can be used. To the extent that a truncated portion of the intracellular signaling domain is used, such truncated portion may be used in place of the entire intracellular signaling domain as long as it transduces the effector function signal. The term intracellular signaling domain is thus meant to include any truncated portion of the intracellular signaling domain sufficient to transduce the effector function signal.

In some embodiments binding of the ligand to the chimeric receptor expressed in an immune cell activates the signaling domain, and the activated signaling domain induces and/or enhances an immune cell function including, without limitation, cell survival of the immune cell, proliferation of the immune cell, migration of the immune cell, or functionality of the immune cell. In some embodiments, signaling is induced through mutimerization or clustering of the chimeric receptors upon binding to ligand. In some embodiments, signaling is induced when multiple copies of the ligand are present, such as at protofibrillar or fibrillar aggregates of Abeta in Alzheimer's disease. In some embodiments, ligand binding and subsequent signaling through the chimeric receptors may be involved in survival and localization of immune cells at cites of pathology occurring in neurological diseases, disorders, or injury.

Examples of intracellular signaling domains for use in the chimer receptor include the cytoplasmic sequences of the T cell receptor (TCR) and co-receptors that act in concert to initiate signal transduction following receptor engagement, as well as any derivative or variant of these sequences and any recombinant sequence that has the same functional capability. It is known that signals generated through the TCR alone are insufficient for full activation of the cell and that a secondary and/or costimulatory signal is also required. Thus, cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. Examples of ITAM containing primary intracellular signaling domains that may be used in the chimeric receptors disclosed herein include those of CD3 zeta, common FcR gamma (FCERIG), Fc gamma Rlla, FcR beta (Fc Epsilon Rib), CD3 gamma, CD3 delta, CD3 epsilon, CD79a, CD79b, DAP10, and DAP12. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs. Further examples of molecules containing a primary intracellular signaling domain for use in the chimeric receptors disclosed herein include those of DAP10, DAP12, and CD32.

The intracellular signaling domain of the chimeric receptor can comprise the CD3-zeta signaling domain by itself or it can be combined with any other desired intracellular signaling domain(s). For example, the intracellular signaling domain of the chimeric receptor can comprise a CD3 zeta chain portion and a costimulatory signaling domain. The costimulatory signaling domain refers to a portion of the chimeric receptor comprising the intracellular domain of a costimulatory molecule. As used herein, a costimulatory molecule refers to the cognate binding partner on a cell that specifically binds with a costimulatory ligand, thereby mediating a costimulatory response by the cell. Costimulatory molecules are cell surface molecules other than antigen receptors or their ligands that contribute to an efficient immune response. Examples of such costimulatory molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83. Further examples of such costimulatory molecules include CD8, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In some embodiments, the signaling domain is from one or more proteins including, without limitation, a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-KappaB, a TLR (toll-like receptor), TLR5, Myd88, target of rapamycin (TOR)/CD3 complex, lymphocyte receptor chain, IL-2 receptor, IgE, IgG, CD16α, FcγRIII, FcγRII, CD28, or 4-1BB. In some embodiments, the signaling domain selected from a 4-1BB intracellular domain, a CD3-zeta ITAM domain, a CD3-zeta intracellular domain, a CSF-1R receptor tyrosine kinase (RTK) intracellular domain, a DAP12 intracellular domain, a TCR-zeta intracellular domain, a TLR5 intracellular domain, a CD28 intracellular domain, a DAP10 intracellular domain, or an FcR-gamma intracellular domain.

Signaling through DAP12 or TCR3Zeta receptor ITAM intracellular domains leads to downstream signaling events such as Syk kinase activation, which promotes survival, functionality, phagocytosis, and proliferation in cells (Turnbull and Colonna, Nat Rev Immunol, 155-161, 2007) (Poliani, Wang et al., J Clin Invest, 2161-2170, 2015) (Wang, Ou j et al., Zhongguo Shi Yan Xue Ye Xue Za Zhi, 568-572, 2015). Major signaling pathways that lead to cell survival are derived from CSF1R and other receptor tyrosine kinase family members such as Kit, the TREM receptor family, and other signaling pathways such as PI3K/AKT. CSF1R and other tyrosine receptor kinase (RTK) activation lead to a pro-survival and proliferation signal for microglia and other immune and/or support cells in the brain, such as astrocytes (Hamilton, Nat Rev Immunol, 533-544, 2008). Furthermore, TREM2, an endogenous receptor that signals through DAP12, is protective in human and in mouse models of Alzheimer's disease and multiple sclerosis.

The intracellular signaling sequences within the cytoplasmic portion of the chimeric receptor may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine-serine doublet can be used as a suitable linker. In one embodiment, a single amino acid, e.g., an alanine, a glycine, can be used as a suitable linker. In one embodiment, the intracellular signaling domain is designed to comprise two or more, e.g., 2, 3, 4, 5, or more, costimulatory signaling domains. hi an embodiment, the two or more, e.g., 2, 3, 4, 5, or more, costimulatory signaling domains, are separated by a linker molecule, e.g., a linker molecule described herein.

Two-Component Chimeric Receptors

In some embodiments, a chimeric receptor of the present disclosure can be a two-component receptor. Two-component chimeric receptors include two separate polypeptides that can associate, dimerize, or multimerize through an interaction domain. In some embodiments, the chimeric receptor comprises a heterodimerization domain, such as an inducible heterodimerization domain. This two-component approach allows one component to harbor a ligand binding domain, together with a linker, transmembrane domain, and inducible heterodimerization domain, and the second polypeptide to harbor a transmembrane domain along with signaling domains and an inducible heterodimerization domain. In some embodiments, one or more signaling components can be located on one of the two-components whereas other signaling domains are located on the other component.

The components can be delivered via two lentiviral vectors or by transfection and selection using two selectable markers. In a exemplary embodiment of a two-component receptor system, a host cell contains (e.g., has been transduced with): (1) a vector containing a polynucleotide that encodes an extracellular ligand-binding domain, wherein the ligand is an agent associated with a neurological disease, disorder, or injury; a flexible linker; a transmembrane domain, and a heterodimerization domain; and (2) a second vector containing a second polynucleotide encoding: a flexible linker, a transmembrane domain, a signaling domain, and a heterodimerization domain. Upon addition of a dimerization-inducing agent, signaling is enhanced due to dimerization or multimerization of both components.

The chimeric receptor can be expressed constitutively after transfer or inducibly to allow for regulation. Induction can be achieved through induced expression using a doxycycline responsive promoter vector or through small molecule-induced receptor dimerization, such as with rapamycin or Rapasyn, a rapamycin analog that is less immunosuppressive. Such an inducible system can allow for limiting the receptor activation period, and/or limiting the location of receptor activation so as to minimize toxicity and maximize dosing. In some embodiments, the inducible heterodimerization domain is a FK506 binding protein (FKBP) heterodimerization domain. In some embodiments, the inducible heterodimerization domain is a T2089L mutant of FKBP-rapamycin binding domain (FRB*) heterodimerization domain.

In another exemplary embodiment of a two-component receptor system, an isolated cell contains (1) a first polynucleotide encoding a chimeric receptor, wherein the chimeric receptor comprises an extracellular ligand-binding domain, wherein the ligand is an agent associated with a neurological disease, disorder, or injury, a flexible linker, a transmembrane domain, and a heterodimerization domain; and (2) a second polynucleotide encoding a flexible linker, a transmembrane domain, a signaling domains, and a heterodimerization domain. In some embodiments, the ligand-binding domain of the chimeric receptor is a single-chain Fv domain (scFv), the agent associated with a neurological disease, disorder, or injury of the chimeric receptor is amyloid beta, the flexible linker of the chimeric receptor is a CD8 hinge domain, the transmembrane domain of the chimeric receptor is a CD8 transmembrane domain, and the heterodimerization domain of the chimeric receptor is an inducible FK506 binding protein (FKBP) heterodimerization domain. In some embodiments, the flexible linker encoded by the second polynucleotide is a CSF-1R linker domain, the transmembrane domain encoded by the second polynucleotide is a CSF-1R 1 transmembrane domain, the one or more signaling domains encoded by the second polynucleotide are a CSF-1R receptor tyrosine kinase (RTK) intracellular domain and a CD3-zeta ITAM domain, and the heterodimerization domain encoded by the second polynucleotide is an inducible T2089L mutant of FKBP-rapamycin binding domain (FRB*) heterodimerization domain. In some embodiments, the first polynucleotide and the second polynucleotide each encode a polypeptide further comprising a CD8 secretory signal peptide at the N-terminus of the encoded polypeptide. In this example, upon addition of rapamycin, FKBP binds FRB*, resulting in association of the first and second components of the two-component chimeric receptor.

### Functional Activities of Chimeric Receptors

In some embodiments, binding of the ligand to the chimeric receptor expressed in an immune cell activates the signaling domain, and the activated signaling domain induces and/or enhances one or more activities, including, without limitation, cell survival of the immune cell, proliferation of the immune cell, migration of the immune cell, phagocytosis, or functionality of the immune cell. These activities can include, without limitation, TREM1 or DAP12 phosphorylation; activation of one or more tyrosine kinases; activation of phosphatidylinositol 3-kinase (PI3K); activation of protein kinase B; recruitment of phospholipase C-gamma (PLC-gamma) to a cellular plasma membrane; activation of PLC-gamma; recruitment of TEC-family kinase dVav to a cellular plasma membrane; activation of nuclear factor-kB (NF-kB), inhibition of MAPK signaling; phosphorylation of linker for activation of T cells (LAT) or linker for activation of B cells (LAB); activation of IL-2-induced tyrosine kinase (Itk); modulation of one or more pro-inflammatory mediators; modulation of one or more anti-inflammatory mediators; phosphorylation of extracellular signal-regulated kinase (ERK); modulated expression of C-C chemokine receptor 7 (CCR7); induction of microglial cell chemotaxis toward CCL19 and CCL21 expressing cells; normalization of disrupted ITAM-dependent gene expression; recruitment of Syk, ZAP70, or both to an ITAM complex; increased activity of one or more ITAM-dependent genes or CSF-1R-dependent genes; increased maturation or survival of dendritic cells, monocytes, microglia, M1 microglia, activated M1 microglia, and M2 microglia, macrophages, M1 macrophages, activated M1 macrophages, M2 macrophages, astrocytes, A1 astrocytes, or A2 astrocytes; increased ability of dendritic cells, monocytes, microglia, M1 microglia, activated M1 microglia, and M2 microglia, macrophages, M1 macrophages, activated M1 macrophages, M2 macrophages, astrocytes, A1 astrocytes, or A2 astrocytes, to prime or modulate the function of T cells; enhanced or normalized ability of bone marrow-derived dendritic cells to prime or modulate function of antigen-specific T cells; modulate function of B cells, modulate antibody production by B cells; induction of osteoclast production; increased rate of osteoclastogenesis; increasing phagocytosis by dendritic cells, macrophages, M1 macrophages, activated M1 macrophages, M2 macrophages, monocytes, microglia, M1 microglia, activated M1 microglia, M2 microglia, astrocytes, A1 astrocytes, A2 astrocytes; induction of one or more types of clearance including apoptotic neuron clearance, nerve tissue debris clearance, non-nerve tissue debris clearance, bacteria clearance, other foreign body clearance, disease-causing protein clearance, disease-causing peptide clearance, disease-causing nucleic acid clearance; induction of phagocytosis of one or more of apoptotic neurons, nerve tissue debris, non-nerve tissue debris, dysfunctional synapses, bacteria, other foreign bodies, disease-causing proteins, disease-causing peptides, disease-causing nucleic acids; increased expression of one or more stimulatory molecules; modulated expression of one or more proteins; increased memory; and reduced cognitive deficit.

TREM2 and/or DAP12 Phosphorylation

In some embodiments, binding of the ligand to the chimeric receptor expressed in the immune cell may induce TREM2 phosphorylation after binding to a TREM2 and/or DAP12 protein expressed by a cell. In other embodiments, binding of the ligand to the chimeric receptor expressed in the immune cell may induce DAP12 phosphorylation after binding to a TREM2 and/or DAP12 protein expressed in a cell. In other embodiments, TREM2 and/or DAP12 phosphorylation is induced by one or more SRC family tyrosine kinases. Examples of Src family tyrosine kinases include, without limitation, Src, Yes, Fyn, Fgr, Lck, Hck, Blk, Lyn, and Frk.

DAP12 is variously referred to as TYRO protein tyrosine kinase-binding protein, TYROBP, KARAP, and PLOSL. DAP12 is a transmembrane signaling protein that contains an immunoreceptor tyrosine-based activation motif (ITAM) in its cytoplasmic domain. In certain embodiments, binding of the ligand to the chimeric receptor expressed in the immune cell may induce DAP12 phosphorylation in its ITAM motif. Any method known in the art for determining protein phosphorylation, such as DAP12 phosphorylation, may be used.

In some embodiments, DAP12 is phosphorylated by SRC family kinases, resulting in the recruitment and activation of the Syk kinase, ZAP70 kinase, or both, to DAP12. Thus, in certain embodiments, the binding of the ligand to the chimeric receptor expressed in the immune cell may recruit Syk, ZAP70, or both to a DAP12/TREM2 complex.

Without wishing to be bound by theory, it is believed that binding of the ligand to the chimeric receptor expressed in the immune cell may be useful for preventing, lowering the risk of, or treating conditions and/or diseases associated with decreased levels of DAP12 activity, DAP12 phosphorylation, or recruitment of Syk, ZAP70, or both to a DAP12/TREM2 complex, including neurological diseases, disorders, or injury.

PI3K Activation

In some embodiments, binding of the ligand to the chimeric receptor expressed in the immune cell may induce may induce PI3K activation in a cell.

PI3Ks are a family of related intracellular signal transducer kinases capable of phosphorylating the 3-position hydroxyl group of the inositol ring of phosphatidylinositol (PtdIns). The PI3K family is divided into three different classes (Class I, Class II, and Class III) based on primary structure, regulation, and in vitro lipid substrate specificity.

Activated PI3K produces various 3-phosphorylated phosphoinositides, including without limitation, PtdIns3P, PtdIns(3,4)P2, PtdIns(3,5)P2, and PtdIns(3,4,5)P3. These 3-phosphorylated phosphoinositides function in a mechanism by which signaling proteins are recruited to various cellular membranes. These signaling proteins contain phosphoinositide-binding domains, including without limitation, PX domains, pleckstrin homology domains (PH domains), and FYVE domains. Any method known in the art for determining PI3K activation may be used.

Without wishing to be bound by theory, it is believed that binding of the ligand to the chimeric receptor expressed in the immune cell may be beneficial for preventing, lowering the risk of, or treating conditions and/or diseases associated with decreased levels of PI3K activity, including neurological diseases, disorders, or injury.

Modulated Expression of Anti-Inflammatory Mediators

In some embodiments, binding of the ligand to the chimeric receptor expressed in the immune cell may modulate (e.g. increase or decrease) anti-inflammatory activities. In certain embodiments, binding of the ligand to the chimeric receptor expressed in the immune cell increases or decreases the expression of anti-inflammatory mediators (e.g., cytokines) and/or modulates the expression of pro-inflammatory mediators.

Inflammation is part of a complex biological response of vascular tissues to harmful stimuli, such as pathogens, damaged cells, and irritants. The classical signs of acute inflammation are pain, heat, redness, swelling, and loss of function. Inflammation is a protective attempt by an organism to remove the injurious stimuli and to initiate the healing process. Inflammation can be classified as either acute inflammation or chronic inflammation. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Chronic inflammation is prolonged inflammation that leads to a progressive shift in the type of cells present at the site of inflammation and is characterized by simultaneous destruction and healing of the tissue from the inflammatory process.

As used herein, anti-inflammatory mediators are proteins involved either directly or indirectly (e.g., by way of an anti-inflammatory signaling pathway) in a mechanism that reduces, inhibits, or inactivates an inflammatory response. Any method known in the art for identifying and characterizing anti-inflammatory mediators may be used. Examples of anti-inflammatory mediators include, without limitation, cytokines, such as IL-4, IL-10, TGF-β, IL-13, IL-35, IL-16, IFN-α, IL-1Rα, VEGF, G-CSF, soluble receptors for TNF, and soluble receptors for IL-6.

In some embodiments, binding of the ligand to the chimeric receptor expressed in the immune cell may modulate expression of anti-inflammatory mediators, such as IL-4, IL-10, TGF-β, IL-13, IL-35, IL-16, IFN-α, IL-1Rα, VEGF, G-CSF, soluble receptors for TNF, and soluble receptors for IL-6. In certain embodiments, modulated expression of the anti-inflammatory mediators occurs in macrophages, dendritic cells, and/or microglial cells. Modulated expression may include, without limitation, modulated in gene expression, modulated transcriptional expression, or modulated protein expression. Any method known in the art for determining gene, transcript (e.g., mRNA), and/or protein expression may be used. For example, Northern blot analysis may be used to determine anti-inflammatory mediator gene expression levels, RT-PCR may be used to determine the level of anti-inflammatory mediator transcription, and Western blot analysis may be used to determine anti-inflammatory mediator protein levels.

As used herein, an anti-inflammatory mediator may have increased expression if its expression in one or more cells expressing a chimeric receptor of the present disclosure is greater than the expression of the same anti-inflammatory mediator expressed in one or more cells that is not expressing a chimeric receptor. In some embodiments, binding of the ligand to the chimeric receptor expressed in the immune cell may increase anti-inflammatory mediator expression in one or more cells by at least 10%, at least 50%, at least 100%, or at least 200% for example, as compared to anti-inflammatory mediator expression in one or more cells that does not express a chimeric receptor. In other embodiments, binding of the ligand to the chimeric receptor expressed in the immune cell increases anti-inflammatory mediator expression in one or more cells by at least 1.5 fold, at least 2.0 fold, or at least 10 fold, for example, as compared to anti-inflammatory mediator expression in one or more cells that does not express a chimeric receptor.

As used herein, an anti-inflammatory mediator may have decreased expression if its expression in one or more cells expressing a chimeric receptor of the present disclosure is less than the expression of the same anti-inflammatory mediator expressed in one or more cells that is not expressing a chimeric receptor. In some embodiments, binding of the ligand to the chimeric receptor expressed in the immune cell may decrease anti-inflammatory mediator expression in one or more cells by at least 10%, at least 50%, at least 100%, or at least 200% for example, as compared to anti-inflammatory mediator expression in one or more cells that does not express a chimeric receptor. In other embodiments, binding of the ligand to the chimeric receptor expressed in the immune cell decreases anti-inflammatory mediator expression in one or more cells by at least 1.5 fold, at least 2.0 fold, or at least 10 fold, for example, as compared to anti-inflammatory mediator expression in one or more cells that does not express a chimeric receptor.

Without wishing to be bound by theory, it is believed that, in some embodiments, binding of the ligand to the chimeric receptor expressed in the immune cell is useful for preventing, lowering the risk of, or treating conditions and/or diseases associated with decreased or increased levels of one or more anti-inflammatory mediators, including neurological diseases, disorders, or injury.

Modulated Expression of Pro-Inflammatory Mediators

In some embodiments, binding of the ligand to the chimeric receptor expressed in the immune cell may modulate (e.g. increase or decrease) the expression of pro-inflammatory mediators in a cell.

As used herein, pro-inflammatory mediators are proteins involved either directly or indirectly (e.g., by way of pro-inflammatory signaling pathways) in a mechanism that induces, activates, promotes, or otherwise increases an inflammatory response. Any method known in the art for identifying and characterizing pro-inflammatory mediators may be used. Examples of pro-inflammatory mediators include, without limitation, cytokines, such as IFN-γ, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, and MCP-1.

In some embodiments, binding of the ligand to the chimeric receptor expressed in the immune cell may modulate functional expression and/or secretion of pro-inflammatory mediators, such as IFN-γ, IL-1α, IL-1β, TNF-α, IL-6, IL-8, CRP, IL-20 family members, IL-33, LIF, IFN-gamma, gamma, OSM, CNTF, GM-CSF, IL-11, IL-12, IL-17, IL-18, IL-23, CXCL10, and MCP-1. In certain embodiments, modulated expression of the pro-inflammatory mediators occurs in macrophages, dendritic cells, monocytes, osteoclasts, Langerhans cells of skin, Kupffer cells, and/or microglial cells. Modulated expression may include, without limitation, modulated gene expression, modulated transcriptional expression, or modulated protein expression. Any method known in the art for determining gene, transcript (e.g., mRNA), and/or protein expression may be used. For example, Northern blot analysis may be used to determine pro-inflammatory mediator gene expression levels, RT-PCR may be used to determine the level of pro-inflammatory mediator transcription, and Western blot analysis may be used to determine pro-inflammatory mediator protein levels.

In certain embodiments, pro-inflammatory mediators include inflammatory cytokines. Accordingly, in certain embodiments, binding of the ligand to the chimeric receptor expressed in the immune cell may reduce secretion of one or more inflammatory cytokines. Examples of inflammatory cytokines whose secretion may be modulated by binding of the ligand to the chimeric receptor expressed in the immune cell may include, without limitation, IFN-γ, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, MCP-1.

As used herein, a pro-inflammatory mediator may have increased expression if its expression in one or more cells of a subject expressing a chimeric receptor of the present disclosure is higher than the expression of the same pro-inflammatory mediator expressed in one or more cells that does not express a chimeric receptor. In some embodiments, the binding of the ligand to the chimeric receptor expressed in the immune cell may increase pro-inflammatory mediator expression in one or more cells by at least 10%, at least 50%, at least 100%, or at least 200% for example, as compared to pro-inflammatory mediator expression in one or more cells that does not express a chimeric receptor. In other embodiments, binding of the ligand to the chimeric receptor expressed in the immune cell may increase pro-inflammatory mediator expression in one or more cells by at least at least 1.5 fold, at least 2.0 fold, or at least 10 fold, for example, as compared to pro-inflammatory mediator expression in one or more cells that does not express a chimeric receptor.

As used herein, a pro-inflammatory mediator may have decreased expression if its expression in one or more cells of a subject expressing a chimeric receptor of the present disclosure is less than the expression of the same pro-inflammatory mediator expressed in one or more cells that does not express a chimeric receptor. In some embodiments, the binding of the ligand to the chimeric receptor expressed in the immune cell may decrease pro-inflammatory mediator expression in one or more cells by at least 10%, at least 50%, at least 100%, or at least 200% for example, as compared to pro-inflammatory mediator expression in one or more cells that does not express a chimeric receptor. In other embodiments, binding of the ligand to the chimeric receptor expressed in the immune cell may decrease pro-inflammatory mediator expression in one or more cells by at least at least 1.5 fold, at least 2.0 fold, or at least 10 fold, for example, as compared to pro-inflammatory mediator expression in one or more cells that does not express a chimeric receptor.

Without wishing to be bound by theory, it is believed that binding of the ligand to the chimeric receptor expressed in the immune cell may be useful for preventing, lowering the risk of, or treating conditions and/or diseases associated with increased levels of one or more pro-inflammatory mediators, including neurological diseases, disorders, or injury.

ERK Phosphorylation

In some embodiments, binding of the ligand to the chimeric receptor expressed in the immune cell may induce extracellular signal-regulated kinase (ERK) phosphorylation.

Extracellular-signal-regulated kinases (ERKs) are widely expressed protein kinase intracellular signaling kinases that are involved in, for example, the regulation of meiosis, mitosis, and postmitotic functions in differentiated cells. Various stimuli, such as growth factors, cytokines, virus infection, ligands for heterotrimeric G protein-coupled receptors, transforming agents, and carcinogens, activate ERK pathways. Phosphorylation of ERKs leads to the activation of their kinase activity.

Without wishing to be bound by theory, it is believed that binding of the ligand to the chimeric receptor expressed in the immune cell is beneficial for preventing, lowering the risk of, or treating conditions and/or diseases associated with decreased levels of ERK phosphorylation, including neurological diseases, disorders, or injury.

Modulated Expression of C-C Chemokine Receptor 7

In some embodiments, binding of the ligand to the chimeric receptor expressed in the immune cell may modulate expression of C-C chemokine receptor 7 (CCR7). Modulated expression may include, without limitation, modulated gene expression, modulated transcriptional expression, or modulated protein expression. Any method known in the art for determining gene, transcript (e.g., mRNA), and/or protein expression may be used. For example, Northern blot analysis may be used to determine gene expression levels, RT-PCR may be used to determine the level of transcription, and Western blot analysis may be used to determine protein levels.

C-C chemokine receptor 7 (CCR7) is a member of the G protein-coupled receptor family. CCR7 is expressed in various lymphoid tissues and can activate B-cells and T-cells. In some embodiments, CCR7 may modulate the migration of memory T-cells to secondary lymphoid organs, such as lymph nodes. In other embodiments, CCR7 may stimulate dendritic cell maturation. CCR7 is a receptor protein that can bind the chemokine (C-C motif) ligands CCL19/ELC and CCL21.

As used herein, CCR7 may have increased expression if its expression in one or more cells expressing a chimeric receptor of the present disclosure is greater than the expression of CCR7 expressed in one or more cells that does not express a chimeric receptor. In some embodiments, binding of the ligand to the chimeric receptor expressed in the immune cell may increase CCR7 expression in one or more cells by at least 10%, at least 50%, at least 100%, or at least 200% for example, as compared to CCR7 expression in one or more cells that does not express a chimeric receptor. In other embodiments, binding of the ligand to the chimeric receptor expressed in the immune cell increases CCR7 expression in one or more cells by at least 1.5 fold, at least 2.0 fold, or at least 10 fold, for example, as compared to CCR7 expression in one or more cells that does not express a chimeric receptor.

As used herein, CCR7 may have decreased expression if its expression in one or more cells expressing a chimeric receptor of the present disclosure is lower than the expression of CCR7 expressed in one or more cells that does not express a chimeric receptor. In some embodiments, binding of the ligand to the chimeric receptor expressed in the immune cell may decrease CCR7 expression in one or more cells by at least 10%, at least 50%, at least 100%, or at least 200% for example, as compared to CCR7 expression in one or more cells that does not express a chimeric receptor. In other embodiments, binding of the ligand to the chimeric receptor expressed in the immune cell decreases CCR7 expression in one or more cells by at least 1.5 fold, at least 2.0 fold, or at least 10 fold, for example, as compared to CCR7 expression in one or more cells that does not express a chimeric receptor.

In some embodiments, modulated expression of CCR7 occurs in macrophages, dendritic cells, and/or microglial cells. Increased expression of CCR7 may induce microglial cell chemotaxis toward cells expressing the chemokines CCL19 and CCL21. Accordingly, in certain embodiments, binding of the ligand to the chimeric receptor expressed in the immune cell may induce microglial cell chemotaxis toward CCL19 and CCL21 expressing cells.

Without wishing to be bound by theory, it is believed that, in some embodiments, binding of the ligand to the chimeric receptor expressed in the immune cell is useful for preventing, lowering the risk of, or treating conditions and/or diseases associated with decreased levels of CCR7, including neurological diseases, disorders, or injury.

Enhanced Ability or Normalized Ability of Cells to Prime or Modulate Function of Antigen-Specific T Cells

In some embodiments, binding of the ligand to the chimeric receptor expressed in the immune cell may enhance and/or normalize the ability of dendritic cells (e.g. bone marrow-derived dendritic cells), monocytes, microglia, M1 microglia, activated M1 microglia, and M2 microglia, macrophages, M1 macrophages, activated M1 macrophages, M2 macrophages, astrocytes, A1 astrocytes, A2 astrocytes to prime or modulate antigen-specific T-cells. T cell priming occurs upon first contact of a T cell with its specific antigen. T cell priming involves antigen uptake, processing, and cell surface expression bound to class II MHC molecules by an antigen presenting cell such as a dendritic cell, recirculation and antigen-specific trapping of helper T cell precursors in lymphoid tissue. T cell priming subsequently results in proliferation and differentiation of naïve T cells into effector T cells. In some embodiments, binding of the ligand to the chimeric receptor expressed in the immune cell may enhance and/or normalize the ability of bone marrow-derived dendritic cells to induce antigen-specific T-cell proliferation.

In some embodiments, binding of the ligand to the chimeric receptor expressed in the immune cell may enhance and/or normalize the ability of dendritic cells (e.g. bone marrow-derived dendritic cells), monocytes, microglia, M1 microglia, activated M1 microglia, and M2 microglia, macrophages, M1 macrophages, activated M1 macrophages, M2 macrophages, astrocytes, A1 astrocytes, A2 astrocytes to induce antigen-specific T-cell proliferation by at least 10%, at least 50%, at least 100%, or at least 200% for example, as compared to the ability of cells that do not contain a chimeric antigen receptor to induce antigen-specific T-cell proliferation. In other embodiments, binding of the ligand to the chimeric receptor expressed in the immune cell may enhance and/or normalize the ability of dendritic cells (e.g. bone marrow-derived dendritic cells), monocytes, microglia, M1 microglia, activated M1 microglia, and M2 microglia, macrophages, M1 macrophages, activated M1 macrophages, M2 macrophages, astrocytes, A1 astrocytes, A2 astrocytes to induce antigen-specific T-cell proliferation by at least at least 1.5 fold, at least 2.0 fold, or at least 10 fold, for example, as compared to the ability of cells that do not contain a chimeric receptor to induce antigen-specific T-cell proliferation.

Without wishing to be bound by theory, it is believed that binding of the ligand to the chimeric receptor expressed in the immune cell is beneficial for preventing, lowering the risk of, or treating conditions and/or diseases associated with an decreased or dysregulated ability of dendritic cells (e.g. bone marrow-derived dendritic cells), monocytes, microglia, M1 microglia, activated M1 microglia, and M2 microglia, macrophages, M1 macrophages, activated M1 macrophages, M2 macrophages, astrocytes, A1 astrocytes, A2 astrocytes to prime or modulate function of antigen-specific T cells, including neurological diseases, disorders, or injury.

Osteoclast Production and Osteoclastogenesis

In some embodiments, binding of the ligand to the chimeric receptor expressed in the immune cell may induce osteoclast production and/or increase the rate of osteoclastogenesis.

As used herein, an osteoclast is a type of bone cell that can remove bone tissue by removing its mineralized matrix and breaking up the organic bone (e.g., bone resorption). Osteoclasts can be formed by the fusion of cells of the monocyte-macrophage cell line. In some embodiments, osteoclasts may be characterized by high expression of tartrate resistant acid phosphatase (TRAP) and cathepsin K.

As used herein, the rate of osteoclast production or osteoclastogenesis may be increased if if the rate of osteoclast production or osteoclastogenesis in a subject treated with chimer receptor-expressing cells of the present disclosure is greater than the rate of osteoclast production or osteoclastogenesis in a corresponding subject that is not treated with chimeric receptor-expressing cells. In some embodiments, binding of the ligand to the chimeric receptor expressed in the immune cell may increase the rate of osteoclastogenesis in a subject by at least 10%, at least 50%, at least 100%, or at least 200% for example, as compared to rate of osteoclast production or osteoclastogenesis in a corresponding subject that is not treated with chimeric receptor-expressing cells. In other embodiments, binding of the ligand to the chimeric receptor expressed in the immune cell may increase the rate of osteoclast production or osteoclastogenesis in a subject by at least 1.5 fold, at least 2.0 fold, or at least 10 fold, for example, as compared to rate of osteoclast production or osteoclastogenesis in a corresponding subject that is not treated with chimeric receptor-expressing cells.

Without wishing to be bound by theory, it is believed that binding of the ligand to the chimeric receptor expressed in the immune cell is beneficial for preventing, lowering the risk of, or treating conditions and/or diseases associated with a reduction in osteoclast production and/or the rate of osteoclastogenesis, including neurological diseases, disorders, or injury.

Function, Maturation, and Survival of Macrophages, Microglial Cells, Dendritic Cells Monocytes, Astrocytes, Osteoclasts, Langerhans Cells of Skin, T Cells, B Cells, and Kupffer Cells

In some embodiments, binding of the ligand to the chimeric receptor expressed in the immune cell may increase the function, maturation, survival, and/or function of dendritic cells, B cells, T cells, macrophages, M1 macrophages, activated M1 macrophages, M2 macrophages, monocytes, osteoclasts, Langerhans cells, Kupffer cells, microglia, M1 microglia, activated M1 microglia, M2 microglia, astrocytes, A1 astrocytes, and A2 astrocytes.

In some embodiments, binding of the ligand to the chimeric receptor expressed in the immune cell may increase the expression of one or more stimulatory molecules selected from CD83, CD86, MHC class II, and CD40 on macrophages, microglial cells, dendritic cells monocytes, astrocytes, osteoclasts, Langerhans cells of skin, and Kupffer cells.

As used herein, the function, maturation, survival, and/or function of macrophages, microglial cells, dendritic cells, B cells, T cells, monocytes, astrocytes, osteoclasts, Langerhans cells of skin, and Kupffer cells may include increased proliferation, maturation, survival, and/or function of macrophages, microglial cells, dendritic cells, B cells, T cells, monocytes, astrocytes, osteoclasts, Langerhans cells of skin, and Kupffer cells in a subject treated with chimeric receptor-expressing cells of the present disclosure compared to the level of proliferation, maturation, survival, and/or function of macrophages, microglia, dendritic cells monocytes, osteoclasts, Langerhans cells of skin, and/or Kupffer cells in a corresponding subject that is not treated with the chimeric receptor-expressing cells. In some embodiments, binding of the ligand to the chimeric receptor expressed in the immune cell may increase proliferation, maturation, survival, and/or function of macrophages, microglial cells, dendritic cells, B cells, T cells, monocytes, astrocytes, osteoclasts, Langerhans cells of skin, and Kupffer cells in a subject by at least 10%, at least 50%, at least 100%, or at least 200% for example, as compared to the proliferation, maturation, survival, and/or function of macrophages, microglial cells, dendritic cells, B cells, T cells, monocytes, astrocytes, osteoclasts, Langerhans cells of skin, and Kupffer cells in a corresponding subject that is not treated with the chimeric receptor-expressing cells. In other embodiments, binding of the ligand to the chimeric receptor expressed in the immune cell may increase proliferation, maturation, survival, and/or function of macrophages, microglial cells, dendritic cells, B cells, T cells, monocytes, astrocytes, osteoclasts, Langerhans cells of skin, and Kupffer cells in a subject by at least 1.5 fold, at least 2.0 fold, or at least 10 fold, for example, as compared to the proliferation, maturation, survival, and/or function of macrophages, microglial cells, dendritic cells, B cells, T cells, monocytes, astrocytes, osteoclasts, Langerhans cells of skin, and Kupffer cells in a corresponding subject that is not treated with chimeric receptor-expressing cells.

Without wishing to be bound by theory, it is believed that binding of the ligand to the chimeric receptor expressed in the immune cell is beneficial for preventing, lowering the risk of, or treating conditions and/or diseases associated with a reduction in proliferation, maturation, survival, and/or function of macrophages, microglial cells, dendritic cells monocytes, astrocytes, osteoclasts, Langerhans cells of skin, and Kupffer cells, including neurological diseases, disorders, or injury.

Clearance and Phagocytosis

In some embodiments, binding of the ligand to the chimeric receptor expressed in the immune cell may induce clearance and/or phagocytosis of one or more agents associated with a neurological disease, disorder, or injury. Exemplary agents that may be phagocytosed or cleared include, without limitation, an apoptotic neuron, nerve tissue debris, non-nerve tissue debris, bacteria, other foreign body, disease-causing protein, disease-causing peptide, and disease-causing nucleic acid. Disease causing proteins include amyloid beta, oligomeric amyloid beta, amyloid beta plaques, amyloid precursor protein or fragments thereof, Tau, LAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products. Disease-causing peptides include DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides. An exemplary disease-causing nucleic acid is antisense GGCCCC (G2C4) repeat-expansion RNA.

In some embodiments, binding of the ligand to the chimeric receptor expressed in the immune cell may induce phagocytosis of one or more of apoptotic neurons, nerve tissue debris, non-nerve tissue debris, bacteria, other foreign bodies, disease-causing proteins, disease-causing peptides, or disease-causing nucleic acid.

In some embodiments, phagocytosis by dendritic cells, macrophages, M1 macrophages, activated M1 macrophages, M2 macrophages, monocytes, microglia, M1 microglia, activated M1 microglia, M2 microglia, astrocytes, A1 astrocytes, or A2 astrocytes is increased. In some embodiments, binding of the ligand to the chimeric receptor expressed in the immune cell may increase phagocytosis by macrophages, dendritic cells, monocytes, and/or microglia under conditions of reduced levels of macrophage colony-stimulating factor (MCSF). Alternatively, in some embodiments, binding of the ligand to the chimeric receptor expressed in the immune cell may decrease phagocytosis by macrophages, dendritic cells, monocytes, and/or microglia in the presence of normal levels of macrophage colony-stimulating factor (MCSF).

Without wishing to be bound by theory, it is believed that binding of the ligand to the chimeric receptor expressed in the immune cell is beneficial for preventing, lowering the risk of, or treating conditions and/or diseases associated with apoptotic neurons, nerve tissue debris of the nervous system, non-nerve tissue debris of the nervous system, bacteria, other foreign bodies, or disease-causing proteins, including neurological diseases, disorders, or injury.

Kinase Activation and Phosphorylation

In some embodiments, binding of the ligand to the chimeric receptor expressed in the immune cell may induce activation or phosphorylation of one or more kinases (e.g. tyrosine kinase, spleen tyrosine kinase (Syk), protein kinase B, or IL-2-induced tyrosine kinase (Itk)).

Spleen tyrosine kinase (Syk) is an intracellular signaling molecule that functions downstream of TREM2 by phosphorylating several substrates, thereby facilitating the formation of a signaling complex leading to cellular activation and inflammatory processes.

Protein kinase B is a serine/threonine-specific protein kinase that plays a key role in multiple cellular processes such as glucose metabolism, apoptosis, cell proliferation, transcription and cell migration.

Itk is an intracellular tyrosine kinase expressed in T-cells. Itk may play a role in T-cell proliferation, differentiation, and the development and effector function of Th2 and Th17 cells.

Without wishing to be bound by theory, it is believed that binding of the ligand to the chimeric receptor expressed in the immune cell is beneficial for preventing, lowering the risk of, or treating conditions and/or diseases associated with decreased levels of kinase activation and phosphorylation, including neurological diseases, disorders, or injury.

Modulated Expression of Proteins

In some embodiments, binding of the ligand to the chimeric receptor expressed in the immune cell may modulate expression of C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF. Modulated expression may include, without limitation, modulated gene expression, modulated transcriptional expression, or modulated protein expression. Any method known in the art for determining gene, transcript (e.g., mRNA), and/or protein expression may be used. For example, Northern blot analysis may be used to determine gene expression levels, RT-PCR may be used to determine the level of transcription, and Western blot analysis may be used to determine protein levels.

As used herein, C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF may have increased expression if its expression in one or more cells expressing a chimeric receptor of the present disclosure is greater than the expression of C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expressed in one or more cells that does not express a chimeric receptor. In some embodiments, binding of the ligand to the chimeric receptor expressed in the immune cell may increase C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells by at least 10%, at least 50%, at least 100%, or at least 200% for example, as compared to C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells that does not express a chimeric receptor. In other embodiments, binding of the ligand to the chimeric receptor expressed in the immune cell increases C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells by at least 1.5 fold, at least 2.0 fold, or at least 10 fold, for example, as compared to C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells that does not express a chimeric receptor.

As used herein, C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF may have decreased expression if its expression in one or more cells expressing a chimeric receptor of the present disclosure is lower than the expression of C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expressed in one or more cells that does not express a chimeric receptor. In some embodiments, binding of the ligand to the chimeric receptor expressed in the immune cell may decrease C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells by at least 10%, at least 50%, at least 100%, or at least 200% for example, as compared to C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells that does not express a chimeric receptor. In other embodiments, binding of the ligand to the chimeric receptor expressed in the immune cell decreases C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells by at least 1.5 fold, at least 2.0 fold, or at least 10 fold, for example, as compared to C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF expression in one or more cells that does not express a chimeric receptor.

In some embodiments, modulated expression of C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF occurs in macrophages, dendritic cells, and/or microglial cells.

Without wishing to be bound by theory, it is believed that, in some embodiments, binding of the ligand to the chimeric receptor expressed in the immune cell is useful for preventing, lowering the risk of, or treating conditions and/or diseases associated with dysregulated levels of C1QA, C1QB, C1QC, C1S, C1R, C4, C2, C3, ITGB2, HMOX1, LAT2, CASP1, CSTA, VSIG4, MS4A4A, C3AR1, GPX1, TYROBP, ALOX5AP, ITGAM, SLC7A7, CD4, ITGAX, PYCARD, or VEGF, including neurological diseases, disorders, or injury.

Recruitment of Signaling Components

In some embodiments, binding of the ligand to the chimeric receptor expressed in the immune cell may modulate recruitment of signaling components. In some embodiments the modulated signaling involves recruitment of phospholipase C-gamma (PLC-gamma) to a cellular plasma membrane and subsequent activation of PLC-gamma, recruitment of TEC-family kinase dVav to a cellular plasma membrane, or recruitment of Syk and/or ZAP70 to an ITAM complex. In some embodiments, recruitment to the plasma membrane results in enhanced signaling and increased downstream effector functions.

PLC is a class of membrane-associated enzymes that cleave phospholipids at a point before a phosphate group and are involved in signal transduction pathways. PLC-gamma catalyzes the formation of inositol 1,4,5-trisphosphate and diacylglycerol from phosphatidylinositol 4,5-bisphosphate. This reaction uses calcium as a cofactor and plays an important role in the intracellular transduction of receptor-mediated tyrosine kinase activators.

TEC family kinases are involved in the intracellular signaling mechanisms of cytokine receptors, lymphocyte surface antigens, heterotrimeric G-protein-coupled receptors, and integrin molecules.

ZAP70, a protein-tyrosine kinase, is part of the TCR and plays an important role in T-cell signaling. Upon phosphorylation of ITAMs during intracellular signaling, ZAP-70 is able to bind to CD3-zeta. The tandem SH2-domains of ZAP-70 are engaged by the doubly phosphorylated ITAMs of CD3-zeta, which positions ZAP-70 to phosphorylate the transmembrane protein linker of activated T cells (LAT). Phosphorylated LAT, in turn, serves as a docking site to which a number of downstream signaling proteins bind.

Without wishing to be bound by theory, it is believed that, in some embodiments, binding of the ligand to the chimeric receptor expressed in the immune cell is useful for preventing, lowering the risk of, or treating conditions and/or diseases associated with dysregulated recruitment of signaling pathway components, including neurological diseases, disorders, or injury.

Inhibition of MAPK Signaling

In some embodiments, binding of the ligand to the chimeric receptor expressed in the immune cell may inhibit MAPK signaling in a cell.

MAPK, or mitogen-activated protein kinases, are serine/threonine protein kinases that are involved in propagating signaling pathways directing cellular responses such as cell proliferation, differentiation, and survival. MAPKs are catalytically inactive in their base form, and require phosphorylation in their activation loops to become activated. Mitogens, cytokines, and cellular stresses promote the activation of different MAPK pathways, which in turn phosphorylate and activate downstream signaling mediators.

Inhibited signaling may include, without limitation, decreased gene expression, decreased transcriptional expression, or decreased protein expression. Any method known in the art for determining gene, transcript (e.g., mRNA), and/or protein expression may be used. For example, Northern blot analysis may be used to determine gene expression levels, RT-PCR may be used to determine the level of transcription, and Western blot analysis may be used to determine protein levels.

Without wishing to be bound by theory, it is believed that, in some embodiments, binding of the ligand to the chimeric receptor expressed in the immune cell is useful for preventing, lowering the risk of, or treating conditions and/or diseases associated with dysregulated MAPK signaling, including neurological diseases, disorders, or injury.

Phosphorylation of Linker for Activation of T Cells (LAT) or Linker for Activation of B Cells (LAB)

In some embodiments, binding of the ligand to the chimeric receptor expressed in the immune cell may modulate phosphorylation of LAT or LAB in a cell.

LAT is phosphorylated by ZAP70/Syk protein tyrosine kinases following activation of the TCR signal transduction pathway. LAT localizes to lipid rafts (also known as glycosphingolipid-enriched microdomains or GEMs) and acts as a docking site for SH2 domain-containing proteins. Upon phosphorylation, LAT recruits multiple adaptor proteins and downstream signaling molecules into multimolecular signaling complexes.

LAB, also known as non-T-cell activation linker (NTAL), is expressed in B cells, NK cells, monocytes, and mast cells. NTAL becomes rapidly tyrosine-phosphorylated upon cross-linking of the B cell receptor (BCR) or of high-affinity Fcγ- and Fcε-receptors of myeloid cells and subsequently associates with cytoplasmic signaling molecules. In addition, LAB is required for TREM-2-mediated activation of Erk1/2 and modulates proximal TREM-2 signals, resulting in macrophages with proinflammatory properties.

Without wishing to be bound by theory, it is believed that, in some embodiments, binding of the ligand to the chimeric receptor expressed in the immune cell is useful for preventing, lowering the risk of, or treating conditions and/or diseases associated with dysregulated LAT or LAB phosphorylation, including neurological diseases, disorders, or injury.

Normalization or Increased Activity of ITAM-Dependent Genes or CSF-1R-Dependent Genes

In some embodiments, binding of the ligand to the chimeric receptor expressed in the immune cell may normalize or increase activity of ITAM-dependent or CSF-1R-dependent genes in a cell. In some embodiments, binding of the ligand to the chimeric receptor expressed in the immune cell may normalize disrupted ITAM-dependent and/or CSF-1R-dependent gene expression. ITAM-dependent and CSF-1R-dependent genes that can be modified include, without limitation, PDL-1, PDL-2, ICOS, B7-H3, B7-H4, OX40L, FOXP3, IDO, CD39, CD73, CD80, CD86, CD83, CD11b, CD14, CD33, Siglec-5, Siglec-7, Siglec-9, IFN-gamma, IFN-alpha,IFN-beta, IL-18, IL-12, IL-10, IL-6, IL-2, IL-1 (beta and alpha), TNF-alpha, TGF-beta, IRF1, IRF3, STAT1, STAT3, HIF1-alpha, GMZA, GMZB, GZMH, PRF1, GNLY, CXCL9, CXCL10, CCL5, CX3CL1, CCL2, MADCAM1, ICAM1, VCAM1, VEGF, GMCSF, MCSF, S1c7a2, Cxc19, Serpinb2, Ptgs2, Cxc13, Cd38, Arg1, Mg12, Retn1a, Ear11, Tmem26, Mrc1, Socs2, Ch25h, Chi313, S1c17a2, Flt1, TIM3, LAG3, CD137, GAL9, OX40, GITR, Osteopontin, MID1, AXL, ITGAX, LPL, SPP1, ATP6VoD2, SIGLECH, CD33, TMEM119, EMR1, CDH23, GLO1, and RASGRF2. ITAM-dependent genes that may be downregulated include, without limitation Saa3, Cd38, C1qa, C1u, Cxc110, H2-T10, Cc15, Hpgd, Pyhin1, Emp2, Cx3cr1, Cd86, Abca1, Ifit1, Cc13, Gpr34, Sparc, Cxc19, Cd14, Aoah, Fcgr1, Slfn8, Itga9, I118, Ebi3, Plxdc2, Edn1, Rasgrp3, Socs3, P2ry13, Aif1, Fam26f, Ccr7, Cp, Ltf, Hp, Ang, Cc14, Mmp9, and I16. ITAM-dependent genes that may be upregulated include, without limitation Arhgap22, I17r, Actn1, Kctd12, Lgmn, Fcnb, Chst7, Lmna, Cc119, Parvg, Siglech, K1, Adcyap1r1, Psd, Sphk1, Cts1, Hsd11b1, Tmem47, Lag3, Bcar3, Tmem158, S1c7a5, S1c2a5, Gp9, Cxc111, F1rt2, Vwf, Cc112, Atp6v0a1, P1k2, Ccnd1, Mmp12, Atf3, Myc, and Egr2. ITAM-dependent and CSF-1R-dependent genes that can be modified in M2 macrophages include, without limitation, ACTN1, AMZ1, ATP6V0A1, ATP6V0D2, BCAR3, CD300LD, CD83, CHST7, CLEC10A, CLEC7A, EGR2, EMP2, FLRT2, GNB4, IL6ST, LMNA, MATK, MMP12, MMP9, MRC1, MYC, OLFM1, P2RY1, PLK2, PTGS1, PTPLA, RHOJ, SOCS6, TANC2, TCFEC, TIAM1, TMEM158, and VWF. ITAM-dependent and CSF-1R-dependent genes that can be modified in M1 macrophages include, without limitation, AOAH, ARHGAP24, CCRL2, CD300LF, CD38, CFB, CP, CPD, CXCL10, D14ERD668E, DDX58, DDX60, E030037K03RIK, EBI3, EPB4.1L3, F11R, FAM176B, FAM26F, FPR1, FPR2, GBP6, GNGT2, GPR18, H2-Q6, H2-T10, HERC6, HP, IFI44, IFIT1, IFIT2, IRAK3, ISF20, ISG15, ITGAL, LOC100503664, MARCO, MPA2L, MS4A4C, MX1, NFKBIZ, OASL1, PILR1, PROBE, PSTPIP2, PYHIN1, RSAD2, SAA3, SEPX1, SLFN1, SLFN4, SLFN8, STAT1, STAT2, TLR2, TUBA4A, XAF1, and ZPB1. CSF-1R-dependent genes that may be downregulated include, without limitation Ms4a6b, Mmp12, Selenbp1, Ndrg1, Bnip3, K1klb11, Selenbp2, AW112010, Rgs17, Bnip3, Mrc1, Scd1, Cxcr4, Ero11, Ms4a7, Scd2, Cyp2ab1, Trib3, Ms4a6c, P1ce1, Ms4a4c, Cyp11a1, NA, NA, Tmem71, Earl, Fabp5, Fabp5, 4930583H14Rik, Tcp1112, C3, Mmp13, Ghrh, Prelid2, F10, Ephx1, Lilra5, Aoah, Gpr162, Car6, and I17r. CSF-1R-dependent genes that may be upregulated include, without limitation Snhg8, NA, Dkc1, Ccnd2, NA, Tsr1, Adapt, Snhg1, Ptgs2, Txnip, Mmp8, Met, NA, Ppbp, Epha2, Jag1, NA, Cxc13, NA, Cc17, NA, NA, Id3, Cd207, NA, NA, Id1, Tfrc, TREM1, and Cc112.

Without wishing to be bound by theory, it is believed that, in some embodiments, binding of the ligand to the chimeric receptor expressed in the immune cell is useful for preventing, lowering the risk of, or treating conditions and/or diseases associated with dysregulated ITAM or CSF-1R signaling pathways, including neurological diseases, disorders, or injury.

Increased Memory and Reduced Cognitive Deficit

In some embodiments, binding of the ligand to the chimeric receptor expressed in the immune cell may increase memory and reduce cognitive deficit in a subject. Reduced cognitive deficits can include beneficial impacts on attention span, language, and problem solving and decreased confusion, irritability, aggression, mood swings, trouble with language, and long-term memory loss.

Without wishing to be bound by theory, it is believed that, in some embodiments, binding of the ligand to the chimeric receptor expressed in the immune cell is useful for preventing, lowering the risk of, or treating conditions and/or diseases associated with decreased memory or cognitive deficit, including neurological diseases, disorders, or injury.

### Polynucleotides Encoding Chimeric Receptors

Certain aspects of the present disclosure relate to an isolated polynucleotide encoding a chimeric receptor. The disclosure encompasses a polynucleotide construct comprising sequences of a chimeric receptor, wherein the sequence comprises the nucleic acid sequence of a ligand-binding domain operably linked to the nucleic acid sequence of a transmembrane domain and an intracellular domain. In some embodiments, by fusing a polynucleotide encoding a ligand binding domain to polynucleotides encoding transmembrane and signaling domains, a chimeric gene is obtained which combines a ligand binding site and intracellular signaling components into one continuous chain. In some embodiments the polynucleotide is a DNA polynucleotide. In some embodiments the polynucleotide is a RNA polynucleotide, such as an mRNA polynucleotide.

The polynucleotide sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques.

Alternatively, the polynucleotide of interest can be produced synthetically, rather than cloned.

Several chimeric receptor constructs are described herein, with each utilizing a different combination of ligand-binding, linker, transmembrane, and/or intracellular signaling domains. The chimeric receptors constructs can be used individually or can be used in any combination. The constructs can be introduced into the same cells or can be introduced into a mixed population of cells that express one or the other construct separately. In some embodiments, the chimeric receptors are referred to as synthetic myeloid activating receptor technology (SMART) receptors. Several SMART receptors are described herein. As used herein, the “>>” symbol indicates association between the different chimeric receptor domains. Chimeric receptor domains are listed, in order from 5′→3′ of the polynucleotide sequence, as ligand-binding domain, linker, transmembrane domain, and signaling domain, with each domain separated by the “>>” symbol (e.g. ligand-binding domain>>linker>>transmembrane domain>>signaling domain). “SS” as used herein refers to a signal sequence. “TM” as used herein refers to a transmembrane domain.

In some embodiments, the polynucleotide comprises a nucleic acid sequence selected from SEQ ID NOs: 38-53. In some embodiments, the polynucleotide comprises a nucleic acid sequence with at least about 80%, about 85%, about 90%, about 95%, about 96%, about 97%, about 98%, or about 99% homology to the polynucleotide sequence selected from SEQ ID NOs: 38-53. In some embodiments, the chimeric receptor comprises an amino acid sequence selected from SEQ ID NOs: 22-37, or an amino acid sequence with at least about 95% homology to the amino acid sequence of SEQ ID NOs: 22-37. In some embodiments, the chimeric receptor comprises an amino acid sequence with at least about 80%, about 85%, about 90%, about 95%, about 96%, about 97%, about 98%, or about 99% homology to the amino acid sequence of SEQ ID NOs: 22-37. Certain aspects of the present disclosure relate to an isolated chimeric receptor encoded by the polynucleotide comprises a nucleic acid sequence selected from SEQ ID NOs: 38-53.

In some embodiments, the chimeric receptor is SMART2. SMART2 is composed of the elements CD8SS>>antiAbeta>>CD8Hinge>>CD8TM>>CD3Z ITAM. The amino acid sequence for SMART2 is provided in SEQ ID NO: 22 and the polynucleotide sequence for SMART2 is provided in SEQ ID NO: 38.

In some embodiments, the chimeric receptor is SMART3. SMART3 is composed of the elements CD8SS>>anti-Abeta>>CD8 Hinge>>CD8TM>>CSF1R RTK intracellular domain. The amino acid sequence for SMART3 is provided in SEQ ID NO: 23 and the polynucleotide sequence for SMART3 is provided in SEQ ID NO: 39.

In some embodiments, the chimeric receptor is SMART4. SMART4 is composed of the elements CD8SS>>anti-Abeta>>CD8 Hinge>>CD8TM>>CSF1R RTK intracellular domain>>CD3Zeta ITAM domain. The amino acid sequence for SMART4 is provided in SEQ ID NO: 24 and the polynucleotide sequence for SMART4 is provided in SEQ ID NO: 40.

In some embodiments, the chimeric receptor is SMART5. SMART5 is composed of the elements CD8SS>>anti-Abeta>>CD8 Hinge>>DAP12 transmembrane and full length intracellular domain. The amino acid sequence for SMART5 is provided in SEQ ID NO: 25 and the polynucleotide sequence for SMART5 is provided in SEQ ID NO: 41.

In some embodiments, the chimeric receptor is SMART6. SMART6 is composed of the elements CD8SS>>anti-Tau_NFT>>CD8 Hinge>>DAP12 transmembrane and full length intracellular domain. The amino acid sequence for SMART6 is provided in SEQ ID NO: 26 and the polynucleotide sequence for SMART6 is provided in SEQ ID NO: 42.

In some embodiments, the chimeric receptor is SMART7. SMART7 is composed of the elements CD8SS>>anti-Tau_NFT>>CD8 Hinge>>CD8 transmembrane>TCRzeta intracellular domain. The amino acid sequence for SMART7 is provided in SEQ ID NO: 27 and the polynucleotide sequence for SMART7 is provided in SEQ ID NO: 43.

In some embodiments, the chimeric receptor is SMART8. SMART8 is composed of the elements CD8SS>>anti-Tau_NFT>>CD8 Hinge>>CD8 transmembrane>CSF1R RTK intracellular domain. The amino acid sequence for SMART8 is provided in SEQ ID NO: 28 and the polynucleotide sequence for SMART8 is provided in SEQ ID NO: 44.

In some embodiments, the chimeric receptor is SMART9. SMART9 is composed of the elements CD8SS>>anti-Abeta>>CSF1R linker, Transmembrane and RTK intracellular domain. The amino acid sequence for SMART9 is provided in SEQ ID NO: 29 and the polynucleotide sequence for SMART9 is provided in SEQ ID NO: 45.

In some embodiments, the chimeric receptor is SMART10. SMART10 is composed of the elements CD8SS>>anti-Abeta>>CSF1R Linker, Transmembrane, and RTK Intracellular domain>>CD3Zeta ITAM domain. The amino acid sequence for SMART10 is provided in SEQ ID NO: 30 and the polynucleotide sequence for SMART10 is provided in SEQ ID NO: 46.

In some embodiments, the chimeric receptor is SMART17. SMART17 is composed of the elements CD8SS>>anti-alphaSynuclein>>CD8Hinge>>CD8TM>>CD3Z ITAM. The amino acid sequence for SMART17 is provided in SEQ ID NO: 31 and the polynucleotide sequence for SMART17 is provided in SEQ ID NO: 47.

In some embodiments, the chimeric receptor is SMART18. SMART18 is composed of the elements CD8SS>>anti-aSynuclein>>CD8 Hinge>>CD8TM>>CSF1R RTK intracellular domain. The amino acid sequence for SMART18 is provided in SEQ ID NO: 32 and the polynucleotide sequence for SMART18 is provided in SEQ ID NO: 48.

In some embodiments, the chimeric receptor is SMART19. SMART19 is composed of the elements CD8SS>>anti-AlphaSynuclein>>CD8 Hinge>>DAP12 transmembrane and full length intracellular domains. The amino acid sequence for SMART19 is provided in SEQ ID NO: 33 and the polynucleotide sequence for SMART19 is provided in SEQ ID NO: 49.

In some embodiments, the chimeric receptor is SMART20. SMART20 is a two-component SMART system composed of component A and component B. Component A is composed of a CD8 signal sequence, anti-Abeta scFv, a CD8 hinge and CD8 transmembrane domain, and a 4-1BB signaling domain upstream of a FKBP inducible dimerization domain. Binding to multimerized or aggregated Abeta can lead to dimerization and multimerization of the receptor components including the dimerization domain. The second fragment, component B, is composed of a CD8 signal sequence and hinge domain, a full length DAP12 domain, and a dimerization component FRB+. In the presence of rapsyn, rapamycin, or a similar molecule, components A and B can dimerize, leading to induction of signaling through the SMART receptor. Local application of rapsyn, such as ICV administration, can localize the induction of myeloid cells in the presence of Abeta aggregates or multimers. Co-transduction of the A and B components of SMART20 can be achieved with 2 lentiviral vectors, or by transfection and selection using 2 selectable markers such as puromycin and G418. The amino acid sequence for SMART20A is provided in SEQ ID NO: 34 and the polynucleotide sequence for SMART20A is provided in SEQ ID NO: 50; the amino acid sequence for SMART20B is provided in SEQ ID NO: 35 and the polynucleotide sequence for SMART20B is provided in SEQ ID NO: 51.

In some embodiments, the chimeric receptor is SMART21. SMART21 is a two-component SMART vector, with component A being composed of the elements CD8SS>>>>anti-Abeta>>CD8 Hinge>>CD8TM>>FKBP and component B being composed of the elements CD8SS>>CSF1R linker, transmembrane, and RTK intracellular domain>>FRB+>>CD3Zeta ITAM domain. In the presence of rapsyn or rapamycin and upon recognition of Abeta multimers or aggregates, myeloid cells expressing components A and B can dimerize and induce ITAM and RTK signaling. The amino acid sequence for SMART21A is provided in SEQ ID NO: 36 and the polynucleotide sequence for SMART21A is provided in SEQ ID NO: 52; the amino acid sequence for SMART21B is provided in SEQ ID NO: 37 and the polynucleotide sequence for SMART21B is provided in SEQ ID NO: 53.

In some embodiments, the amino acid sequence of the ligand-binding domain (or other portions or the entire chimeric receptor) can be modified, e.g., an amino acid sequence described herein can be modified, e.g., by a conservative substitution Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

### Vectors

Certain aspects of the present disclosure relate to a vector comprising a polynucleotide encoding a chimeric receptor. In some embodiments, one or more vectors (e.g., expression vectors) containing such polynucleotides are provided.

For recombinant production of a chimeric receptor of the present disclosure, a polynucleotide encoding the chimeric receptor is isolated and inserted into one or more vectors for further cloning and/or expression in a host cell. Such polynucleotides may be readily isolated and sequenced using conventional procedures (e.g., by using oligonucleotide probes that are capable of binding specifically to genes encoding the chimeric receptor domains).

Suitable vectors containing a polynucleotide encoding a chimeric receptor of the present disclosure include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide of the present disclosure. The expression vector may replicable in the host cells either as episomes or as an integral part of the chromosomal DNA. Suitable expression vectors include but are not limited to plasmids, viral vectors, including adenoviruses, adeno-associated viruses (AAVs), lentiviral vectors, retroviral vectors, cosmids, a sleeping beauty vector, non-viral plasmid vectors and expression vector(s) disclosed in PCT Publication No. WO 87/04462. In some embodiments, the vector is pCDNA3.4-Topo from Life Technologies.

Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons. In one embodiment, the nucleic acid sequence in the vector further comprises a poly(A) tail. In one embodiment, the nucleic acid sequence in the vector further comprises a 3′UTR e.g., comprising at least one repeat of a 3′UTR derived from human beta-globulin.

In some embodiment, the vector comprises a promoter. Depending on the promoter, individual elements can function either cooperatively or independently to activate transcription. Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another.

Exemplary promoters for use in the present disclosure include, without limitation, the CMV IE gene, EF-1 promoter, ubiquitin C, phosphoglycerokinase (PGK) promoter, T2A promoter, and thymidine kinase (tk) promoter. In some embodiments, the promoter is an EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving chimeric receptor expression from transgenes cloned into a lentiviral vector. See, e.g., Milone et al., Mol. Ther. 17(8): 1453-1464 (2009). Another example of a promoter is the immediate early cytomegalovirus (CMV IE) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Inducible promoters are also contemplated for use in the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

### Host Cells

Certain aspects of the present disclosure relate to a host cell comprising a chimeric receptor. In some embodiments, a host cell containing a polynucleotide encoding a chimeric receptor is provided. In some embodiments, the host cell is an isolated host cell. As used herein, an “isolated cell” is a cell that is identified and separated from at least one contaminant cell with which it is ordinarily associated in the environment in which it was produced. In some embodiments, the isolated cell is free of association with all components associated with the production environment. The isolated cell is in a form other than in the form or setting in which it is found in nature. Isolated cells are distinguished from cells existing naturally in tissues, organs, or individuals. In some embodiments, the isolated cell is a host cell of the present disclosure. In some embodiments, the host cell contains (e.g., has been transduced with): a vector containing a polynucleotide that encodes an extracellular ligand-binding domain, wherein the ligand is an agent associated with a neurological disease, disorder, or injury; a flexible linker; a transmembrane domain, and a signaling domain. In some embodiments, the host cell is eukaryotic, e.g., a Chinese Hamster Ovary (CHO) cell or lymphoid lymphoid cell (e.g., Y0, NS0, Sp20 cell). Host cells of the present disclosure also include, without limitation, isolated cells, in vitro cultured cells, and ex vivo cultured cells. In some embodiments, a host cell of the present disclosure containing a polynucleotide encoding said chimeric receptor is cultured under conditions suitable for expression of the chimeric receptor.

Immune Cells

In some embodiments, the host cell is an immune cell. Any suitable immune cell known in the art may be used. In some embodiments, the immune cell is an innate immune cell or an adaptive immune cell. Immune cells for use in the present disclosure may be in a resting or activated state. For example, in some embodiments, the immune cell is a cell that has been activated by the presence of antigen, cytokines, or other activating ligands.

In some embodiments, the immune cell is an innate immune cell. In some embodiments, the innate immune cell is a NK cell. NK (natural killer) cells are innate lymphocytes which are differentiated from the common lymphoid progenitor (CLP). NK cells have diverse functions, including recognizing and killing virally-infected and tumor cells (mediated by the contents of their cytotoxic granules) and secreting cytokines such as IFNγ.

In some embodiments, the innate immune cell is a myeloid cell. Myeloid cells are derived from hematopoietic stem cells in the bone marrow. Myeloid cells include megakaryocytes, erythrocyte-precursors, mononuclear phagocytes (monocytes/macrophages) and all of the polymorphonuclear leukocytes (neutrophils, basophils, eosinophils). Exemplary myeloid cells include, without limitation, macrophages, monocytes, neutrophils, dendritic cells (DCs), osteoclasts, Langerhans cells, Kuppfer cells, and microglia.

Monocytes are amoeboid shaped cells with agranulated cytoplasm and unilobar nuclei. Monocytes circulate in the bloodstream and can migrate in response to inflammatory signals. Upon migration from the bloodstream to other tissues, monocytes diffrentiate into tissue resident macrophages or DCs. Functional activities of monocytes, macrophages, and DCs include, without limitation, phagocytosis, antigen presentation, and cytokine production. Neutrophils, which contain a multilobulated nucleus, are recruited to sites of injury or infection by chemotaxis, and function via phagocytosis, release of soluble anti-microbials, and generation of neutrophil extracellular traps (NETs). Langerhans cells are resident dendritic cells of the skin and mucosa. The have a similar morphology and function as macrophages, including antigen presentation. Kupffer cells, also known as stellate macrophages, are resident macrophages of the liver that play a role in host defense and in the homeostatic responses of tissue. Kupffer cells are found in the hepatic sinusoids and function by endocytosing blood-borne materials which enter the liver. Osteoclasts are bone cells that are involved involved in the maintenance, repair, and remodeling of bones. Osteoclasts are derived from the myeloid lineage and are formed in the presence of receptor activator of nuclear factor κβ ligand (RANKL) and macrophage colony-stimulating factor (M-CSF) produced by stromal cells and osteoblasts.

Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most infections from reaching the vulnerable nervous tissue. In the case where infectious agents are directly introduced to the brain or cross the blood-brain barrier, microglial cells must react quickly to decrease inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T-cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

Some aspects of the present disclosure include an isolated myeloid cell comprising a chimeric receptor. In some embodiments, an isolated myeloid cell comprises a first polynucleotide encoding a chimeric receptor, wherein the chimeric receptor comprises an extracellular ligand-binding domain, wherein the ligand is an agent associated with a neurological disease, disorder, or injury; a flexible linker; a transmembrane domain, and a heterodimerization domain; and a second polynucleotide encoding: a flexible linker, a transmembrane domain, a signaling domains, and a heterodimerization domain. In some embodiments, the ligand-binding domain of the chimeric receptor is a single-chain Fv domain (scFv), the agent associated with a neurological disease, disorder, or injury of the chimeric receptor is amyloid beta, the flexible linker of the chimeric receptor is a CD8 hinge domain, the transmembrane domain of the chimeric receptor is a CD8 transmembrane domain, and the heterodimerization domain of the chimeric receptor is an inducible FK506 binding protein (FKBP) heterodimerization domain. In some embodiments, the flexible linker encoded by the second polynucleotide is a CSF-1R linker domain, the transmembrane domain encoded by the second polynucleotide is a CSF-1R 1 transmembrane domain, the one or more signaling domains encoded by the second polynucleotide are a CSF-1R receptor tyrosine kinase (RTK) intracellular domain and a CD3-zeta ITAM domain, and the heterodimerization domain encoded by the second polynucleotide is an inducible T2089L mutant of FKBP-rapamycin binding domain (FRB*) heterodimerization domain. In some embodiments, the first polynucleotide and the second polynucleotide each encode a polypeptide further comprising a CD8 secretory signal peptide at the N-terminus of the encoded polypeptide.

In some embodiments, the cell phenotype of an isolated myeloid cell expressing a chimeric receptor is modified in vitro, ex vivo, or in vivo by addition of pro-inflammatory or anti-inflammatory agents or cytokines. Such cytokines can include, without limitation, GM-CSF, MCSF, IL-1, IL4, IL10, IL12, TNFα, TGF-beta, and LPS.

In some embodiments, the innate immune cell is an astrocyte. Astrocytes, also called astroglia, are specialized glial cells found in the brain and spinal cord. Astrocytes are derived from heterogeneous populations of progenitor cells in the neuroepithelium of the developing central nervous system. Astrocyte functions include endothelial cell support, regulation of blood flow, synapse function, maintenance of extracellular ion balance, and nervous system repair. Astrocytes also function as immune cells in the CNS via their production of cytokines and expression of class II MHC antigens and costimulatory molecules that are critical for antigen presentation and T-cell activation. Astrocytes are also involved in various neurological diseases, including Alzheimer's disease, amyotrophic lateral sclerosis, Parkinson's disease, and dementia. Early stages of neurological disease processes are thought to be associated with atrophy of astroglia, which causes disruptions in synaptic connectivity and neurotransmitter homeostasis, and neuronal death. At the later stages of disease, astrocytes may become activated and contribute to the neuroinflammatory component of neurological diseases. In some embodiments, the astrocyte is an A1 astrocyte or an A2 astrocyte. A1 astrocytes express the adenosine A1 receptor, while A2 astrocytes express the adenosine A2 receptor. Adenosine receptors have inhibitory functions, including decreasing metabolic activity and reducing synaptic vesicle release during nerve transmission.

In some embodiments, the innate immune cell has an M1 or M2 phenotype. In some embodiments, innate immune cells with an M1 phenotype are involved in inflammatory process and may secrete pro-inflammatory cytokines such as IL-1 and TNFα. In some embodiments, innate immune cells with an M2 phenotype are involved in resolution of inflammation and tissue repair. For example, macrophages can be polarized toward a classically activated (M1) phenotype in the presence of lipopolysaccharide (LPS) and IFNγ. M1 macrophages can produce large amounts of the cytokines TNF, IL-12, and IL-23 and can help drive pro-inflammatory and antigen specific T cell responses. Conversely, macrophages can be polarized toward an alternatively activated (M2) phenotype in the presence of IL-4. M2 macrophages can produce large amounts of the cytokines IL-10 and IL-1RA and function in immunoregulation and tissue remodeling. In some embodiments, the innate immune cell is a M1 macrophage, M2 macrophage, M1 microglia, or M2 microglia.

In some embodiments, genetic manipulation of cells encoding chimeric receptors can allow polarization of cells in a directed manner. For example, cells can be polarized toward a protective/regenerative M2-like phenotype or an M1-like pro-inflammatory state through inhibition of components of the NFKappaB complex pathway (e.g. IKK). In some embodiments, the isolated host cell further expresses one or more signaling factors that promote an M2 phenotype by inhibiting a TNF-alpha/NF-KappaB pathway a TLR/MyD88 pathway, or both. Such signaling factors can include, without limitation, dominant negative IKK-alpha, a dominant negative IKK-alpha IKK-beta, a dominant negative IKK-alpha IKBa (IKBa-DN), a MEKK isoform, and any combination thereof. In some embodiments, the one or more signaling factors that promote an M2 phenotype by inhibiting a TLR/MyD88 pathway are one or more dominant negative forms of MyD88.

In some embodiments the immune cell is an adaptive immune cell. Adaptive immune cells include, without limitation, T cells, T helper cells, cytotoxic T cells, memory T cells, regulatory T cells, natural killer T cells, mucosal associate invariant T cells, gamma delta T cells, B cells, memory B cells, follicular B cells, marginal zone B cells, B-1 cells, B-2 cells, and regulatory B cells.

Methods of Producing Chimeric Receptor-Expressing Cells

Certain aspects of the present disclosure include a method of producing an immune cell expressing a chimeric receptor. The vectors containing the polynucleotides encoding a chimeric receptor can be introduced into a host cell by any of a number of appropriate means. Vectors can be transferred to a host cell in vitro, ex vivo, or in vivo. For example, the expression vector can be transferred into a host cell by physical, chemical, or biological means. The choice of introducing vectors or polynucleotides will often depend on features of the host cell.

Physical methods for introduction include calcium phosphate precipitation, lipofection, particle bombardment, microinjection, and electroporation, and the like. Methods for producing cells comprising vectors and/or exogenous nucleic acids are well-known in the art. In some embodiments, the polynucleotide is introduced into a host cell by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g. an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the polynucleotides or vectors into a host cell (in vitro, ex vivo or in vivo). In another aspect, the polynucleotide may be associated with a lipid. The polynucleotide or vector associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce polynucleotides into a host cell, in order to confirm the presence of the polynucleotide in the host cell, a variety of assays may be performed. Such assays include, for example, “molecular biological” assays well known to those of skill in the art, such as Southern and Northern blotting, RT-PCR and PCR;

Or “biochemical” assays, such as detecting the presence or absence of a particular peptide, e.g., by immunological means (ELISAs and Western blots).

In some embodiments, an immune cell expressing a chimeric receptor is produced by isolating an immune cell, introducing a vector encoding a chimeric receptor, and culturing the cell so that the chimeric receptor is expressed. In some embodiments, vector constructs expressing a chimeric receptor can be directly transduced into a cell. In some embodiments, an RNA construct encoding a chimeric receptor can be directly transfected into a cell. Upon transfection or transduction of such chimeric receptor-encoding polynucleotides into immune cells, the construct is expressed in the cell as a functional receptor and endows the cells with ligand specificity.

Allogeneic Cells

In some embodiments, the immune cell is an allogeneic cell. In some embodiments, the immune cell is modified to be an allogeneic cell. In some embodiments, the immune cell may be modified to lack one or more genes encoding one or more immune molecules that allow for recognition by the adaptive immune system. For example, heterologous chimeric receptor-expressing cells, such as from unrelated individuals or relatives, can be modified so as to minimize potential immunogenicity. Exemplary immune recognition molecules include, without limitation, MHC class I molecules, MHC class I co-receptors, MHC class II molecules, MHC class II co-receptors, HLA class I molecules, or HLA class II molecules.

In some embodiments, the allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a chimer receptor-expressing cell to mount an immune effector response. Examples of inhibitory molecules include PD1, PD-L1, CTLA-4, TIM3, CEACAM (e.g., CEACAM-1, CEACAM-3 and/or CEACAM-5), LAG3, VISTA, BTLA, TIGIT, LAIR1, CD160, 2B4 and TGFR beta. Inhibition of an inhibitory molecule, e.g., by inhibition at the DNA, RNA or protein level, can optimize a chimeric receptor-expressing cell's function.

Allogeneic cells that lack expression of a functional MHC or HLA can be obtained by any suitable means, including a knock out or knock down of one or more subunit of HLA or MHC. For example, the cell can include a knock down of MHC or HLA using siRNA, shRNA, clustered regularly interspaced short palindromic repeats (CRISPR) transcription-activator like effector nuclease (TALEN), or zinc finger endo nuclease (ZFN). In some embodiments, an inhibitory nucleic acid, e.g., an inhibitory nucleic acid, e.g., a sRNA, e.g., an siRNA or shRNA, can be used.

In some embodiments, MHC or HLA expression can be inhibited using siRNA or shRNA. that targets a nucleic acid encoding a MHC or HLA in a cell. Expression of siRNA and shRNAs in T cells can be achieved using any conventional expression system, e.g., such as a lentiviral expression system.

CRISPR, as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a MHC or HLA gene. The CRISPR/Cas system has been modified for use in gene editing (silencing, enhancing or changing specific genes) in eukaryotes such as mice or primates. This is accomplished by introducing into the cell a plasmid containing a specifically designed CRISPR and one or more appropriate Cas. The CRISPR/Cas system can thus be used to edit a gene (adding or deleting a basepair), or introducing a premature stop which thus decreases expression of a MHC or HLA. The CRISPR/Cas system can alternatively be used like RNA interference, turning off WIC or HLA gene in a reversible fashion. in a mammalian cell, for example, the RNA can guide the Cas protein to a MHC or HLA promoter, sterically blocking RNA polymerases.

A TALEN protein is a transcription activator-like effector nuclease, an artificial nuclease which can be used to edit the MHC or HLA gene. TALENs are produced artificially by fusing a TAL effector DNA binding domain to a DNA cleavage domain. They can be engineered to bind any desired desired DNA sequence, including a portion of the HLA or MHC gene. By combining an engineered TALE with a DNA cleavage domain, a restriction enzyme can be produced which is specific to any desired DNA sequence, including a HLA or MHC sequence. These can then be introduced into a cell, wherein they can be used for genome editing.

ZFNs are artificial nucleases which can be used to edit the HLA and/or MHC gene. Like a TALEN, a ZEN comprises a Fold nuclease domain (or derivative thereof) fused to a DNA-binding domain. In the case of a ZEN, the DNA-binding domain comprises one or more zinc fingers and must dimerize to cleave DNA, The two individual ZFNs must bind opposite strands of the DNA with their nucleases properly spaced apart, A ZEN can create a double-stranded break in the DNA, which can create a frame-shift mutation if improperly repaired, leading to a decrease in the expression and amount of HLA or MHC in a cell, ZFNs can also be used with homologous recombination to mutate in the HLA or MHC gene.

In some embodiments, genes encoding key immune molecules such as MHC class I and II, the Beta2-Microglobulin component of the MHC class I complex, or the invariant chain component of MHC class II can be mutated or deleted or otherwise rendered dysfunctional using CRISPR-Cas9, TALEN, or Zinc Finger nucleases. Vectors to deliver CRISPR-Cas9, TALEN, Zinc Finger nucleases or similar reagents can be transfected or transduced in the cells, or these factors could be introduced as RNA or proteins. Immune cells can then be screened or purified for the loss of expression of the immune molecules, such as MCH class I or II. Such methods may reduce the potential for antigenicity of the introduced chimeric receptor-expressing cells in the context of heterologous treatment therapies.

### Pharmaceutical Compositions

Certain aspects of the present disclosure relate to pharmaceutical compositions comprising polynucleotides, vectors, or cells encoding chimeric receptors and a pharmaceutically acceptable carrier. Polynucleotides, vectors, or cells encoding the chimeric receptors of the present disclosure can be incorporated into a variety of formulations for therapeutic administration by combining the polynucleotides, vectors, or cells with appropriate pharmaceutically acceptable carriers carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the polynucleotides, vectors, or cells encoding chimeric receptors of the present disclosure, in a biodegradable or bioerodible implant. The rate of release of the therapeutically active agent is controlled by the rate of transport through the polymeric matrix, and the biodegradation of the implant. The transport of agent through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions that may be employed include, without limitation, organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject disclosures. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical Dosages

Pharmaceutical compositions of the present disclosure containing polynucleotides, vectors, or cells encoding chimeric receptors of the present disclosure may be administered to an individual in need of treatment, preferably a human, in accord with known methods, such as intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes.

Dosages and desired concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the polynucleotides, vectors, or cells encoding chimeric receptors of the present disclosure, normal dosage amounts may vary depending on an individual's body weight and upon the route of administration. For repeated administrations over several days or longer, depending on the severity of the disease, disorder, or condition to be treated, the treatment is sustained until a desired suppression of symptoms is achieved.

Different dosage regimens may be useful, depending on the pattern of pharmacokinetic decay that the physician wishes to achieve. For example, dosing an individual from one to twenty-one one times a week is contemplated herein. In certain embodiments, dosing frequency is three times per per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the polynucleotides, vectors, or cells encoding chimeric receptors administered, can vary over time independently of the dose used.

Dosages for particular polynucleotides, vectors, or cells encoding chimeric receptors may be determined empirically in individuals who have been given one or more administrations of the polynucleotides, vectors, or cells encoding chimeric receptors. Individuals are given incremental doses of polynucleotides, vectors, or cells encoding chimeric receptors. To assess efficacy of polynucleotides, vectors, or cells encoding chimeric receptors, a clinical symptom of any of the diseases, disorders, or conditions of the present disclosure (e.g., neurological diseases, disorders, or injury) can be monitored.

Administration of polynucleotides, vectors, or cells encoding chimeric receptors of the present disclosure can be continuous or intermittent, depending, for example, on the recipient's physiological condition, whether the purpose of the administration is therapeutic or prophylactic, and other factors known to skilled practitioners. The administration of polynucleotides, vectors, or cells encoding chimeric receptors may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the disclosures that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Therapeutic Uses

The immune cells expressing a chimeric receptor of the present disclosure may be used in therapeutic treatment processes. Not to be bound by theory, neurological disease, disorder, or injury pathology could be suppressed by introducing modified immune cells that express a chimeric receptor and are programmed to be activated appropriately and selectively only in the presence of neurological disease, disorder, or injury pathology. Immune cells may be modified to express a chimeric receptor in vitro, ex vivo, or in vivo.

In some embodiments, a plurality of isolated immune cells expressing chimeric receptors is administered to a patient. In some embodiment the plurality of isolated immune cells is administered peripherally. In some embodiments, the plurality of isolated immune cells is administered peripherally into the individual without irradiation. In some embodiments, the plurality of isolated immune cells is administered directly to the CNS. In some embodiments, the administered plurality of isolated immune cells infiltrates the central nervous system of the individual after administration.

In some embodiments, the isolated immune cells are autologous cells. For example, the immune cells may be obtained from a subject in need of treatment, modified to express a chimeric receptor of the present disclosure, and transferred back into the same individual. In some embodiments, the cells are from an allogeneic donor. For example, the cells may be obtained from a different individual, modified to express a chimeric receptor, and transferred into a subject in need of treatment. In some embodiments, the allogeneic cells are modified to lack one or more genes encoding one or more immune molecules that allow for recognition by the adaptive immune system. For example, heterologous chimeric receptor-expressing cells, such as from unrelated individuals or relatives, can be modified so as to minimize potential immunogenicity. Exemplary immune recognition molecules include that can be modified include, without limitation, HLA class I molecules and HLA class II molecules.

In some embodiments, a source of cells is obtained from a subject prior to modification (e.g. delivery of a polynucleotide encoding a chimeric receptor). Cells can be obtained from a number of sources, including peripheral blood mononuclear cells, bone marrow, lymph node tissue, cord blood, thymus tissue, tissue from a site of infection, ascites, pleural effusion, spleen tissue, and brain. In some embodiments, cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps.

Some embodiments of the present disclosure involved a method of preventing, reducing risk, or treating a neurological disease, disorder, or injury in an individual. For example, the method may include obtaining a plurality of isolated immune cells, introducing a vector containing polynucleotides encoding a chimeric receptor into the plurality of isolated immune cells, and administering to the individual a therapeutically effective amount of the plurality of isolated immune cells containing the vector. In some embodiments, myeloid cells isolated from a patient may be transfected with a polynucleotide encoding a chimeric receptor directed toward a disease-associated ligand and then returned to the patient so that the cellular response generated by such cells is triggered. In some embodiments, the vector contained in the plurality of isolated immune cells is expressed after administration of the plurality of immune cells to the individual.

In some embodiments, a specific cell subpopulation can be selected prior to modification (e.g. delivery of a polynucleotide encoding a chimeric receptor). For example, cells can be immunolabeled by staining with antibodies for specific cell surface markers. The immunolabeled cells can then be subjected to selection via positive or negative of specific subpopulations. Techniques to select specific subpopulations include, without limitation, bead-based selection and fluorescence-activated cell sorting (FACS). For example, enrichment of a cell population by negative selection can be accomplished, e.g., with a combination of antibodies directed to surface markers unique to the negatively selected cells. Cell sorting and/or selection via negative magnetic immunoadherence or flow cytometry may be subsequently used to achieve enrichment of the desired cell population. Some embodiments include therapeutic delivery of cells to a central nervous system for preventing, reducing risk, or treating a neurological disease, disorder, or injury in an individual in need thereof by obtaining a blood sample, a bone marrow sample, or a brain tissue sample from a suitable donor, immunolabeling a plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells in the blood sample, the bone marrow sample, or the brain tissue sample, isolating the immunolabeled plurality of cells from the blood sample, the bone marrow sample, or the brain tissue sample, introducing the vector containing polynucleotides encoding a chimeric receptor into the isolated plurality of cells, and administering to the periphery of the individual, without irradiation of the individual or any portion of the individual, a therapeutically effective amount of the plurality of cells containing the vector, wherein the administered plurality of cells infiltrates the central nervous system of the individual.

In some embodiments, the expression of the vector in the administered cells increases recruitment of the cells across the blood-brain barrier and into the central nervous system of the individual. In some embodiments, expression of the vector in the administered cells has one of more therapeutic effects in the central nervous system. For example, in some embodiments, the administered cells recruit myeloid cells having an M2-like protective phenotype or an A2-like protective phenotype into the central nervous system of the individual. In some embodiments, the cells induce production of endogenous neuronal survival agents, trophic agents, or both locally in the central nervous system of the individual. In some embodiments, binding of the ligand to the chimeric receptor induces an increase in myeloid cell activation, proliferation, survival, phagocytosis, and/or functionality.

In some embodiments, administering the immune cells containing a chimeric receptor or polynucleotides encoding said receptor induces one or more activities, including without limitation, TREM1 or DAP12 phosphorylation, activation of one or more kinases, modulated signaling pathways, modulated expression or one or more proteins, modulation of one or more pro-inflammatory or anti-inflammatory mediators, modulated expression of C-C chemokine receptor 7 (CCR7), induction of microglial cell chemotaxis toward CCL19 and CCL21 expressing cells, maturation, function, or survival of dendritic cells, monocytes, microglia, macrophages, astrocytes, osteoclasts, Langerhans cells, or Kupffer cells, modified osteoclast production or rate of osteoclastogenesis, induction of clearance or phagocytosis of disease-associated factors (e.g. protein, nucleic acids, or cells), increased expression of one or more stimulatory molecules, increased memory, and reduced cognitive deficit.

Some embodiments include testing the efficacy of therapeutic delivery of cells to a central nervous system for preventing, reducing risk, or treating a neurological disease, disorder, or injury. For example, the testing may include obtaining a blood sample or a bone marrow sample from a suitable donor; immunolabeling a plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells in the blood sample or the bone marrow sample; isolating the immunolabeled plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells from the blood sample or the bone marrow sample; introducing the vector encoding a chimeric receptor into the isolated plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells; administering to the periphery or brain of the individual, without irradiation of the individual or any portion of the individual, a therapeutically effective amount of the plurality of cells containing the vector, wherein the administered plurality of cells infiltrates the locus of the neurological disease, disorder, or injury within the central nervous system of the individual; detecting the presence of the administered plurality of cells at the locus of the neurological disease, disorder, or injury within the central nervous system of the individual; and determining the therapeutic effect of the administered plurality of cells at the locus of the neurological disease, disorder, or injury.

Certain aspects of the present disclosure provide methods of preventing, reducing risk, or treating a neurological disease, disorder, or injury comprising administering to an individual in need thereof a therapeutically effective amount of an isolated cell containing a chimeric receptor or polynucleotides encoding such chimeric receptors. Exemplary neurological diseases, disorders, or injuries include, without limitation, dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, Taupathy disease, Nasu-Hakola disease, stroke, acute trauma, chronic trauma, lupus, acute and chronic colitis, wound healing, Crohn's disease, inflammatory bowel disease, ulcerative colitis, obesity, Malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, by administering to the individual a therapeutically effective amount of a plurality of immune cells expressing the chimeric receptor. In some embodiments, the neurological disease, disorder, or injury is Alzheimer's disease and the cells infiltrate Amyloid beta plaques in the central nervous system of the individual.

Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, mixed dementia, semantic dementia, and dementia with Lewy bodies.

Without wishing to be bound by theory, it is believed that administering a therapeutically effective amount of a plurality of isolated immune cells expressing a chimeric receptor of the present disclosure can prevent, reduce the risk, and/or treat dementia. In some embodiments, administering a therapeutically effective amount of a plurality of isolated immune cells expressing the chimeric receptor may induce one or more activities in an individual having dementia (e.g., immune cell activation, proliferation, survival, phagocytosis, and/or functionality).

Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the progranulin gene.

Without wishing to be bound by theory, it is believed that administering a therapeutically effective amount of a plurality of isolated immune cells expressing a chimeric receptor of the present disclosure can prevent, reduce the risk, and/or treat FTD. In some embodiments, administering a therapeutically effective amount of the plurality of isolated immune cells expressing the chimeric receptor may induce one or more activities in an individual having FTD (e.g., immune cell activation, proliferation, survival, phagocytosis, and/or functionality).

Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

Without wishing to be bound by theory, it is believed that administering a therapeutically effective amount of a plurality of isolated immune cells expressing a chimeric receptor of the present disclosure can prevent, reduce the risk, and/or treat Alzheimer's disease. In some embodiments, administering a therapeutically effective amount of the plurality of isolated immune cells expressing the chimeric receptor may induce one or more activities in an individual having Alzheimer's disease (e.g., immune cell activation, proliferation, survival, phagocytosis, and/or functionality).

Nasu-Hakola Disease

Nasu-Hakola disease (NHD), which may alternatively be referred to as polycystic lipomembranous osteodysplasia with sclerosing leukoencephalopathy (PLOSL), is a rare inherited leukodystrophy characterized by progressive presenile dementia associated with recurrent bone fractures due to polycystic osseous lesions of the lower and upper extremities. NHD disease course is generally divided into four stages: latent, osseous, early neurologic, and late neurologic. After a normal development during childhood (latent stage), NHD starts manifesting during adolescence or young adulthood (typical age of onset 20-30 years) with pain in the hands, wrists, ankles, and feet. Patients then start suffering from recurrent bone fractures due to polycystic osseous and osteroporotic lesions in the limb bones (osseous stage). During the third or fourth decade of life (early neurologic stage), patients present with pronounced personality changes (e.g., euphoria, lack of concentration, loss of judgment, and social inhibitions) characteristic of a frontal lobe syndrome. Patients also typically suffer from progressive memory disturbances. Epileptic seizures are also frequently observed. Finally (late neurologic stage), patients progress to a profound dementia, are unable to speak and move, and usually die by the age of 50.

Without wishing to be bound by theory, it is believed that administering a therapeutically effective amount of a plurality of isolated immune cells expressing a chimeric receptor of the present disclosure can prevent, reduce the risk, and/or treat NHD. In some embodiments, administering a therapeutically effective amount of the plurality of isolated immune cells expressing the chimeric receptor may induce one or more activities in an individual having NHD (e.g., immune cell activation, proliferation, survival, phagocytosis, and/or functionality).

Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

Without wishing to be bound by theory, it is believed that administering a therapeutically effective amount of a plurality of isolated immune cells expressing a chimeric receptor of the present disclosure can prevent, reduce the risk, and/or treat Parkinson's disease. In some embodiments, administering a therapeutically effective amount of the plurality of isolated immune cells expressing the chimeric receptor may induce one or more activities in an individual having Parkinson's disease (e.g., immune cell activation, proliferation, survival, phagocytosis, and/or functionality).

Amyotrophic Lateral Sclerosis

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

It has been shown that progranulin play a role in ALS (Schymick, J C et al., (2007) J Neurol Neurosurg Psychiatry.; 78:754-6) and protects again the damage caused by ALS causing proteins such as TDP-43 (Laird, A S et al., (2010). PLoS ONE 5: e13368). It was also demonstrated that pro-NGF induces p75 mediated death of oligodendrocytes and corticospinal neurons following spinal cord injury (Beatty et al., Neuron (2002), 36, pp. 375-386; Giehl et al, Proc. Natl. Acad. Sci USA (2004), 101, pp 6226-30).

Without wishing to be bound by theory, it is believed that administering a therapeutically effective amount of a plurality of isolated immune cells expressing a chimeric receptor of the present disclosure can prevent, reduce the risk, and/or treat ALS. In some embodiments, administering a therapeutically effective amount of the plurality of isolated immune cells expressing the chimeric receptor may induce one or more activities in an individual having ALS (e.g., immune cell activation, proliferation, survival, phagocytosis, and/or functionality).

Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

Without wishing to be bound by theory, it is believed that administering a therapeutically effective amount of a plurality of isolated immune cells expressing a chimeric receptor of the present disclosure can prevent, reduce the risk, and/or treat HD. In some embodiments, administering a therapeutically effective amount of the plurality of isolated immune cells expressing the chimeric receptor may induce one or more activities in an individual having HD (e.g., immune cell activation, proliferation, survival, phagocytosis, and/or functionality).

Taupathy Disease

Taupathy diseases, or Tauopathies, are a class of neurodegenerative disease caused by aggregation of the microtubule-associated protein tau within the brain. Alzheimer's disease (AD) is the most well-known Taupathy disease, and involves an accumulation of tau protein within neurons in the form of insoluble neurofibrillary tangles (NFTs). Other Taupathy diseases and disorders include progressive supranuclear palsy, dementia pugilistica (chromic traumatic encephalopathy), Frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, frontotemporal dementia, and frontotemporal lobar degeneration.

Without wishing to be bound by theory, it is believed that administering a therapeutically effective amount of a plurality of isolated immune cells expressing a chimeric receptor of the present disclosure can prevent, reduce the risk, and/or treat Taupathy disease. In some embodiments, administering a therapeutically effective amount of the plurality of isolated immune cells expressing the chimeric receptor may induce one or more activities in an individual having Taupathy disease (e.g., immune cell activation, proliferation, survival, phagocytosis, and/or functionality).

Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

Without wishing to be bound by theory, it is believed that administering a therapeutically effective amount of a plurality of isolated immune cells expressing a chimeric receptor of the present disclosure can prevent, reduce the risk, and/or treat MS. In some embodiments, administering a therapeutically effective amount of the plurality of isolated immune cells expressing the chimeric receptor may induce one or more activities in an individual having MS (e.g., immune cell activation, proliferation, survival, phagocytosis, and/or functionality).

Creutzfeldt-Jakob Disease

Creutzfeldt-Jakob disease (CJD) is a prion disease which has sporadic, iatrogenic, and familial forms. CJD is characterized by spongiform change (e.g., microcavitation of the brain, usually predominant in gray matter), neuronal cell loss, astrocytic proliferation disproportionate to neuronal loss, and accumulation of an abnormal amyloidogenic protein, sometimes in discrete plaques in the brain. Prions, the infectious agents that transmit these diseases differ markedly from viruses and viroids in that no chemical or physical evidence for a nucleic acid component has been reproducibly detected in infectious materials.

Without wishing to be bound by theory, it is believed that administering a therapeutically effective amount of a plurality of isolated immune cells expressing a chimeric receptor of the present disclosure can prevent, reduce the risk, and/or treat CJD. In some embodiments, administering a therapeutically effective amount of the plurality of isolated immune cells expressing the chimeric receptor may induce one or more activities in an individual having CJD (e.g., immune cell activation, proliferation, survival, phagocytosis, and/or functionality).

Normal Pressure Hydrocephalus

Normal pressure hydrocephalus (NPH) is an abnormal buildup of cerebrospinal fluid (CSF) in the brain's ventricles. NPH can be caused by blockages in the normal flow of CSF throughout the brain and spinal cord, resulting in enlarged ventricles and pressure on the brain. It may result from a subarachnoid hemorrhage, head trauma, infection, tumor, or surgical complications. Symptoms of NPH include progressive mental impairment, dementia, problems with walking, and impaired bladder control.

Without wishing to be bound by theory, it is believed that administering a therapeutically effective amount of a plurality of isolated immune cells expressing a chimeric receptor of the present disclosure can prevent, reduce the risk, and/or treat NPH. In some embodiments, administering a therapeutically effective amount of the plurality of isolated immune cells expressing the chimeric receptor may induce one or more activities in an individual having NPH (e.g., immune cell activation, proliferation, survival, phagocytosis, and/or functionality).

Central Nervous System Lupus

Central nervous system (CNS) lupus is a neurologic manifestation of systemic lupus erythematosus (SLE), a multisystem autoimmune connective tissue disorder. CNS lupus is a serious illness with neurologic symptoms which include headaches, confusion, fatigue, depression, seizures, strokes, vision problems, mood swings, and difficulty concentrating.

Without wishing to be bound by theory, it is believed that administering a therapeutically effective amount of a plurality of isolated immune cells expressing a chimeric receptor of the present disclosure can prevent, reduce the risk, and/or treat CNS lupus. In some embodiments, administering a therapeutically effective amount of the plurality of isolated immune cells expressing the chimeric receptor may induce one or more activities in an individual having CNS lupus (e.g., immune cell activation, proliferation, survival, phagocytosis, and/or functionality).

Behcet's Disease

Behcet's (beh-CHETS) disease, also called Behcet's syndrome, is a rare autoinflammatory disorder that causes blood vessel inflammation. The causes of Behcet's disease are unknown, but may be caused by vasculitis-mediated blood vessel damage. Symptoms of Behcet's disease include mouth sores, eye inflammation, skin rashes and lesions, genital sores, arthritis, blood clots, inflammation in the central nervous system, and kidney dysfunction.

Without wishing to be bound by theory, it is believed that administering a therapeutically effective amount of a plurality of isolated immune cells expressing a chimeric receptor of the present disclosure can prevent, reduce the risk, and/or treat Behcet's disease. In some embodiments, administering a therapeutically effective amount of the plurality of isolated immune cells expressing the chimeric receptor may induce one or more activities in an individual having Behcet's disease (e.g., immune cell activation, proliferation, survival, phagocytosis, and/or functionality).

Multiple System Atrophy

Multiple system atrophy (MSA), also known as Shy-Drager syndrome, is a progressive neurodegenerative disorder characterized symptoms that affect both the autonomic nervous system and movement. Symptoms are the result of progressive loss of function and death of different types of nerve cells in the brain and spinal cord and include fainting spells, heart rate problems, and bladder control. Motor impairments include tremor, rigidity, loss of muscle coordination, and difficulties with speech and gait. MSA includes disorders that historically had been referred to as Shy-Drager syndrome, olivopontocerebellar atrophy, and striatonigral degeneration. A distinguishing feature of MSA is the accumulation of the protein alpha-synuclein in glia, the cells that support nerve cells in the brain.

Without wishing to be bound by theory, it is believed that administering a therapeutically effective amount of a plurality of isolated immune cells expressing a chimeric receptor of the present disclosure can prevent, reduce the risk, and/or treat MSA. In some embodiments, administering a therapeutically effective amount of the plurality of isolated immune cells expressing the chimeric receptor may induce one or more activities in an individual having MSA (e.g., immune cell activation, proliferation, survival, phagocytosis, and/or functionality).

Progressive Supranuclear Palsy

Progressive supranuclear palsy (PSP) is an uncommon brain disorder that results from damage to nerve cells in the brain. PSP affects movement, control of walking (gait), balance, speech, swallowing, vision, mood, behavior, thinking, and control of eye movements. The symptoms of PSP are caused by a gradual deterioration of brain cells in a few specific areas in the brain, mainly in the region called the brain stem. PSP is characterized by abnormal deposits of the protein tau in nerve cells in the brain.

Without wishing to be bound by theory, it is believed that administering a therapeutically effective amount of a plurality of isolated immune cells expressing a chimeric receptor of the present disclosure can prevent, reduce the risk, and/or treat PSP. In some embodiments, administering a therapeutically effective amount of the plurality of isolated immune cells expressing the chimeric receptor may induce one or more activities in an individual having PSP (e.g., immune cell activation, proliferation, survival, phagocytosis, and/or functionality).

Cortical Basal Ganglionic Degeneration

Cortical basal ganglionic degeneration (CBGD) is a rare, progressive neurodegenerative disease involving the cerebral cortex and the basal ganglia. CBGD symptoms include movement and cognitive dysfunction, Parkinsonism, alient hand syndrome, and psychiatric disorders. CBGD pathology is characterized by the presence of astrocytic abnormalities within the brain and improper accumulation of the protein tau.

Without wishing to be bound by theory, it is believed that administering a therapeutically effective amount of a plurality of isolated immune cells expressing a chimeric receptor of the present disclosure can prevent, reduce the risk, and/or treat CBGD. In some embodiments, administering a therapeutically effective amount of the plurality of isolated immune cells expressing the chimeric receptor may induce one or more activities in an individual having CBGD (e.g., immune cell activation, proliferation, survival, phagocytosis, and/or functionality).

Acute Disseminated Encephalomyelitis

Acute disseminated encephalomyelitis (ADEM), or acute demyelinating encephalomyelitis, is a rare autoimmune disease characterized by widespread inflammation in the brain and spinal cord. ADEM also damages myelin insulation on nerves of the CNS, destroying the white matter. ADEM is characterized by multiple inflammatory lesions in the subcortical and central white matter and cortical gray-white junction of the cerebral hemispheres, cerebellum, brainstem, and spinal cord.

Without wishing to be bound by theory, it is believed that administering a therapeutically effective amount of a plurality of isolated immune cells expressing a chimeric receptor of the present disclosure can prevent, reduce the risk, and/or treat ADEM. In some embodiments, administering a therapeutically effective amount of the plurality of isolated immune cells expressing the chimeric receptor may induce one or more activities in an individual having ADEM (e.g., immune cell activation, proliferation, survival, phagocytosis, and/or functionality).

Granulomatous Disorders

Granulomatous disorders, such as chronic granulomatous disease (CGD), also known as Bridges-Good syndrome, include disorders in which immune cells have difficulty forming reactive oxygen compounds. Deficiency in production of superoxide radical, due to defective phagocyte NADPH oxidase, leads to an inability to kill certain ingested pathogens and the subsequent formation of granulomata in many organs. Symptoms include pneumonia, abscesses of the skin, tissues, and organs, suppurative arthritis, osteomyelitis, bacteremia/fungemia, and superficial skin infections such as cellulitis or impetigo.

Without wishing to be bound by theory, it is believed that administering a therapeutically effective amount of a plurality of isolated immune cells expressing a chimeric receptor of the present disclosure can prevent, reduce the risk, and/or treat Granulomatous disorders. In some embodiments, administering a therapeutically effective amount of the plurality of isolated immune cells expressing the chimeric receptor may induce one or more activities in an individual having Granulomatous disorders (e.g., immune cell activation, proliferation, survival, phagocytosis, and/or functionality).

Sarcoidosis

Sarcoidosis is a disease involving abnormal collections of inflammatory cells that can form as nodules (granulomas) in multiple organs. Sarcoidosis may be caused by an immune reaction to an infection or innocuous antigen. Symptoms include fatigue, lack of energy, weight loss, joint aches and pains, arthritis, dry eyes, swelling of the knees, blurry vision, shortness of breath, a dry, hacking cough, or skin lesions. Sarcoidosis affecting the nervous system is known as neurosarcoidosis, and most commonly affects the cranial nerves. Manifistations include peripheral facial nerve palsy, optic nerve dysfunction, papilledema, palate dysfunction, neuroendocrine changes, hearing abnormalities, hypothalamic and pituitary abnormalities, chronic meningitis, and peripheral neuropathy.

Without wishing to be bound by theory, it is believed that administering a therapeutically effective amount of a plurality of isolated immune cells expressing a chimeric receptor of the present disclosure can prevent, reduce the risk, and/or treat Sarcoidosis. In some embodiments, administering a therapeutically effective amount of the plurality of isolated immune cells expressing the chimeric receptor may induce one or more activities in an individual having Sarcoidosis (e.g., immune cell activation, proliferation, survival, phagocytosis, and/or functionality).

CNS Herpes

CNS herpes, also known as Herpes simplex encephalitis (HSE,) is a life-threatening consequence of herpes simplex virus (HSV) infection of the CNS. Although HSE is rare, mortality rates reach over 70% in the absence of therapy. Symptoms include fever, headache, psychiatric symptoms, seizures, vomiting, focal weakness, and memory loss.

Without wishing to be bound by theory, it is believed that administering a therapeutically effective amount of a plurality of isolated immune cells expressing a chimeric receptor of the present disclosure can prevent, reduce the risk, and/or treat CNS herpes. In some embodiments, administering a therapeutically effective amount of the plurality of isolated immune cells expressing the chimeric receptor may induce one or more activities in an individual having CNS herpes (e.g., immune cell activation, proliferation, survival, phagocytosis, and/or functionality).

Neurological Injury

Neurological injuries can results from stroke, acute trauma, chronic trauma, seizures, spinal cord injury, traumatic brain injury (TBI), alcohol abuse, or vitamin B deficiency. Neurological injuries can result in impairment or disability, including neurocognitive deficits, delusions, speech or movement problems, intellectual disability, sleep disorders, mental fatigue, personality changes, coma or a persistent vegetative state.

Without wishing to be bound by theory, it is believed that administering a therapeutically effective amount of a plurality of isolated immune cells expressing a chimeric receptor of the present disclosure can prevent, reduce the risk, and/or treat neurological injury. In some embodiments, administering a therapeutically effective amount of the plurality of isolated immune cells expressing the chimeric receptor may induce one or more activities in an individual having neurological injury (e.g., immune cell activation, proliferation, survival, phagocytosis, and/or functionality).

### Kits/Articles of Manufacture

The present disclosure also provides kits containing polynucleotides, vectors, or cells encoding chimeric receptors. Kits of the present disclosure may include one or more containers comprising polynucleotides, vectors, or cells encoding chimeric receptors of the present disclosure. In In some embodiments, the kits further include instructions for use in accordance with the methods of this disclosure. In some embodiments, these instructions comprise a description of administration of the polynucleotides, vectors, or cells encoding chimeric receptors of the present disclosure to prevent, reduce risk, or treat an individual having a neurological disease, disorder, or injury, according to any methods of this disclosure. The kit may further comprise a description of selecting an individual suitable for treatment based on identifying whether that individual has the disease and the stage of the disease.

In some embodiments, the kits may further include an additional therapeutic agent. In some embodiments, the kits may further include instructions for using the additional therapeutic agent in combination with the polynucleotides, vectors, or cells encoding chimeric receptors of the present disclosure, according to any methods of this disclosure.

The instructions generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a disease of the present disclosure. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is a polynucleotide, vector, or cell a encoding chimeric receptor. The container may further comprise a second pharmaceutically active agent.

Kits may optionally provide additional components such as buffers and interpretive information. Normally, the kit comprises a container and a label or package insert(s) on or associated with the container.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Assembly, Production, Identification, and Characterization of SMART Vectors

### Introduction

SMART chimeric receptors are composed minimally of a ligand-binding domain such as an scFv, a transmembrane domain, and one or more intracellular signaling domains. The intracellular domain may be from an ITAM protein domain such as those found in TCRzeta or DAP12.

The antigen binding domain may be composed of an scFv, which can be designed by connecting sequences from the heavy chain and light chain of an antibody via a linker domain. An exemplary linker is shown in SEQ ID NO: 12. Another possible linker for use in the SMART chimeric receptors disclosed herein is the sequence GGGGSGGGGSGGGGS (SEQ ID NO: 70), which is exemplified in the scFv sequences displayed in SEQ ID NO: 13, SEQ ID NO: 18, and SEQ ID NO: 19. Examples of complete scFv sequences are provided in SEQ ID NO: 13 (Abeta scFv), SEQ ID NO: 18 (anti-PHF Tau), and SEQ ID NO: 19 (alpha-Synuclein scFv). To generate a SMART vector, these linkers must be preceded by a signal sequence to allow for membrane targeting. An exemplary signal sequence from the CD8 gene is shown in SEQ ID NO: 11. A hinge domain may be added downstream of the scFv, preceding the transmembrane domain, functioning as an additional linker. An exemplary hinge domain is shown in SEQ ID NO: 14 (from CD8), and can be followed by a transmembrane domain as shown in SEQ ID NO: 15 (from CD8).

Intracellular signaling domains are chosen for insertion into SMART vectors depending on the desired chimeric receptor activity. For example, ITAM domain signaling promotes survival and can in some contexts polarize towards an M2-type repair phenotype. An exemplary ITAM signaling domain is the CD3Zeta intracellular domain (shown in SEQ ID NO: 1), which contains 3 ITAM sequences and is predicted to lead to strong signaling. SEQ ID NO: 17, which represents the entire DAP12 molecule minus the signal sequence, is an example of a very short extracellular domain followed by a transmembrane domain and an ITAM domain.

Another exemplary intracellular signaling domain class is derived from receptor tyrosine kinase molecules. One such receptor tyrosine kinase is CSF1R, which can be included as an intracellular domain only (SEQ ID NO: 5), together with the CSF1R transmembrane domain (SEQ ID NO: 4), or as the transmembrane plus extracellular linker/hinge together with the intracellular domain (SEQ ID NO: 3). Such domains can also be derived from other non-human species. Although the use of human derived sequences minimizes antigenicity in humans, sequences derived from non-human species can be used when performing testing in animal models. For example, mouse CSF1R intracellular (SEQ ID NO: 7) and transmembrane domains (SEQ ID NO: 6) can be used.

Signaling domains, such as TLR signaling intracellular domains (SEQ ID NO: 9), may also be used to polarize towards a pro-inflammatory M1-like phenotype. These signaling domains may be used alone, or may be place downstream of a transmemrane domain (SEQ ID NO: 8) and an extracellular linker. Another domain which may lead to pro-inflammatory and survival signaling in myeloid cells is the intracellular domain derived from CD28 (SEQ ID NO: 10). This domain may lead to NFKappaB, Syk, and PI3K signaling induction. Similarly, the 4-1BB intracellular domain (SEQ ID NO: 16), when expressed within SMART vectors in myeloid cells, may lead to pro-inflammatory polarization via induction of NFKappaB and beta-catenin signaling.

Several SMART constructs are described herein, with each utilizing a different combination of antigen binding, linker, transmembrane, and/or intracellular signaling domains. The SMART constructs can be used individually or multiple vectors can be used in any combination. The vectors can be introduced into the same cells or can be introduced into a mixed population of cells that express one or the other vector separately.

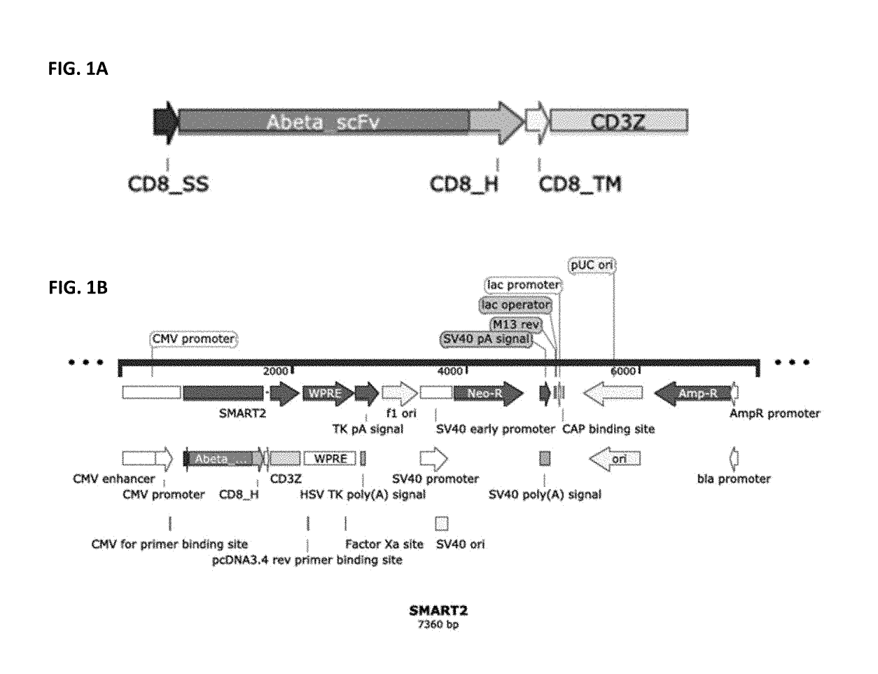

SMART2 is composed of the elements CD8SS>>antiAbeta>>CD8Hinge>>CD8TM>>CD3Z ITAM. The SMART2 construct may lead to ITAM mediated survival and anti-inflammatory/pro-repair signaling in the context of Abeta multimers or aggregates. The sequences for SMART2 are provided in SEQ ID NO: 22 (amino acid) and SEQ ID NO: 38 (polynucleotide), and the vectors are shown in FIG. 1. This vector may allow selective and local cellular activation.

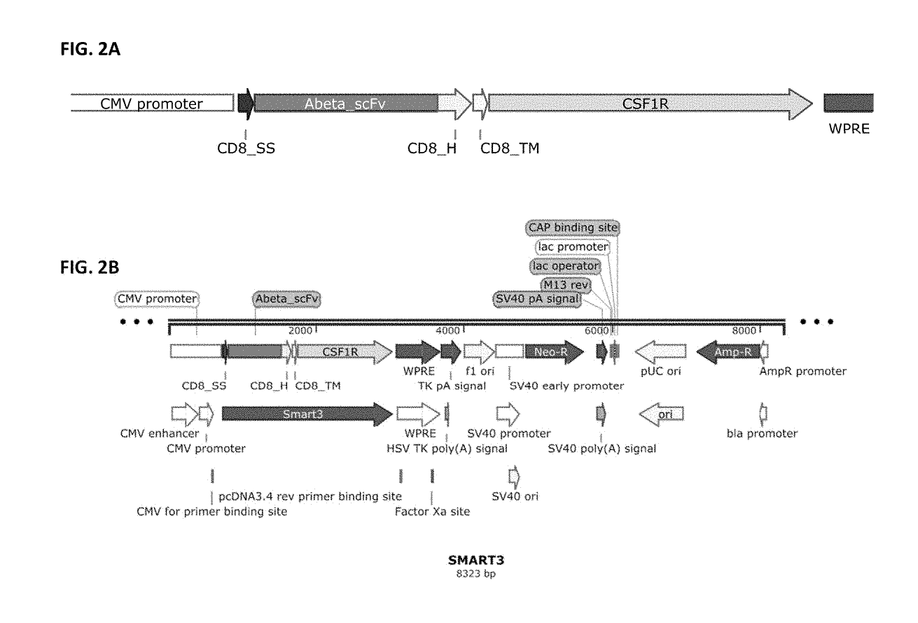

SMART3 is composed of the elements CD8SS>>anti-Abeta>>CD8 Hinge>>CD8TM>>CSF1R RTK intracellular domain. SMART3 may lead to RTK mediated survival and anti-inflammatory/pro-repair signaling in the context of Abeta multimers or aggregates. The sequences for SMART3 are provided in SEQ ID NO: 23 (amino acid) and SEQ ID NO: 39 (polynucleotide), the vectors are shown in FIG. 2. This construct may allow a selective and local activation of RTK activity in the context of Abeta soluble or insoluble multimers at sites of AD pathology.

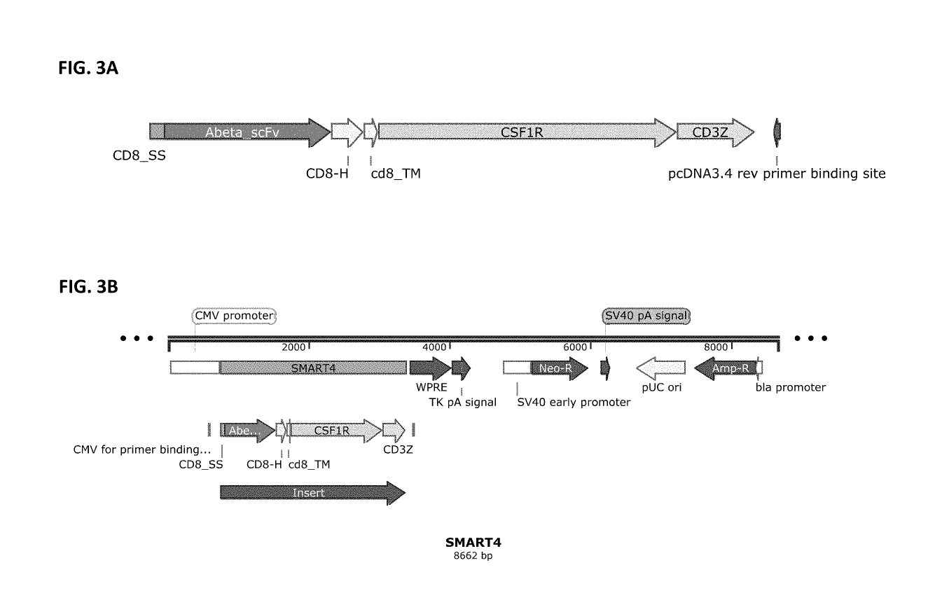

SMART4 is composed of the elements CD8SS>>anti-Abeta>>CD8 Hinge>>CD8TM>>CSF1R RTK intracellular domain>>CD3Zeta ITAM domain. SMART4 may lead to both RTK and ITAM mediated survival and anti-inflammatory/pro-repair signaling in the context of Abeta multimers or aggregates. The sequences for SMART4 are provided in SEQ ID NO: 24 (amino acid) and SEQ ID NO: 40 (polynucleotide), and the vectors are shown in FIG. 3. This construct may allow a selective and local activation of RTK and ITAM activity in the context of Abeta soluble or insoluble multimers at sites of AD pathology.

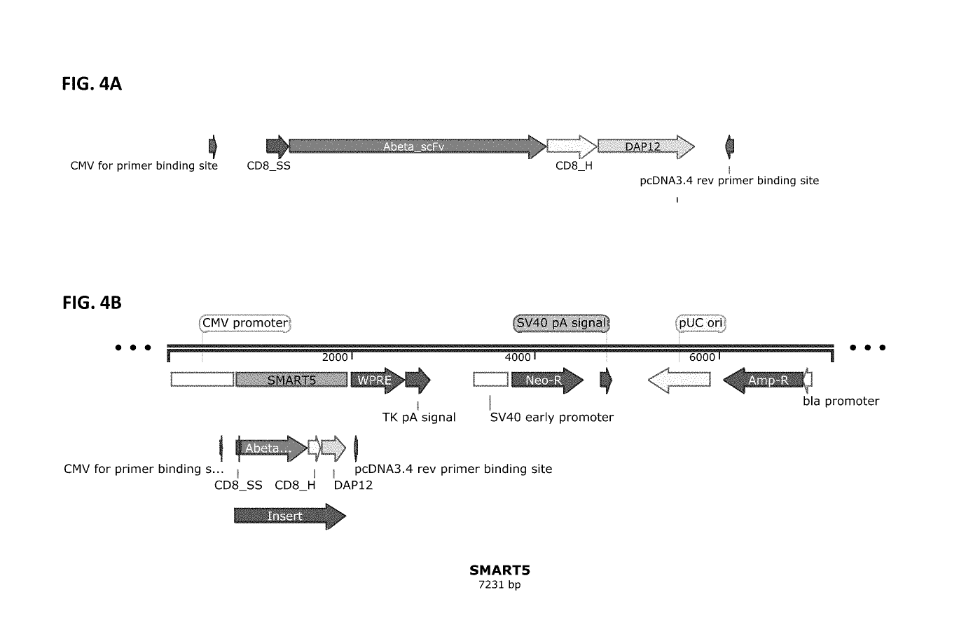

SMART5 is composed of the elements CD8SS>>anti-Abeta>>CD8 Hinge>>DAP12 transmembrane and full length intracellular domain. SMART5 may lead to ITAM mediated survival and anti-inflammatory/pro-repair signaling in the context of Abeta multimers or aggregates. The ITAM for DAP12 is normally expressed in myeloid cells, unlike CD3Z, and thus may lead to a more selective activation in these cells than SMART3. The sequences for SMART5 are provided in SEQ ID NO: 25 (amino acid) and SEQ ID NO: 41 (polynucleotide), and the vectors are shown in FIG. 4. This construct may allow a selective and local activation of ITAM activity in the context of Abeta soluble or insoluble multimers at sites of AD pathology.

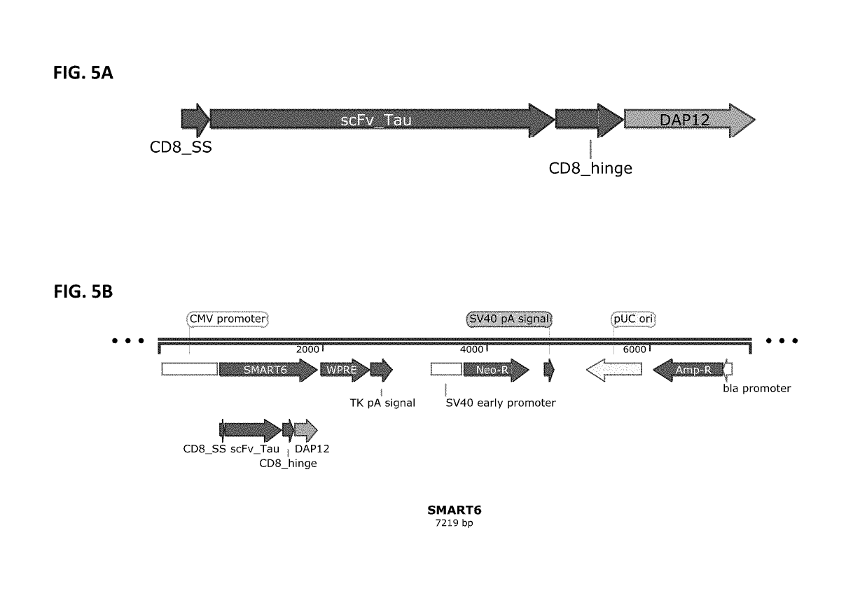

SMART6 is composed of the elements CD8SS>>anti-Tau_NFT>>CD8 Hinge>>DAP12 transmembrane and full length intracellular domain. SMART6 may lead to ITAM mediated survival and anti-inflammatory/pro-repair signaling in the context of Tau multimers or aggregates. The ITAM for DAP12 is normally expressed in myeloid cells, unlike CD3Z, and thus may lead to a more selective activation in these cells than SMART3. The sequences for SMART5 are provided in SEQ ID NO: 26 (amino acid) and SEQ ID NO: 42 (polynucleotide), and the vectors are shown in FIG. 5. This construct may allow a selective and local activation of ITAM activity in the context of Tau soluble or insoluble multimers at sites of AD pathology.

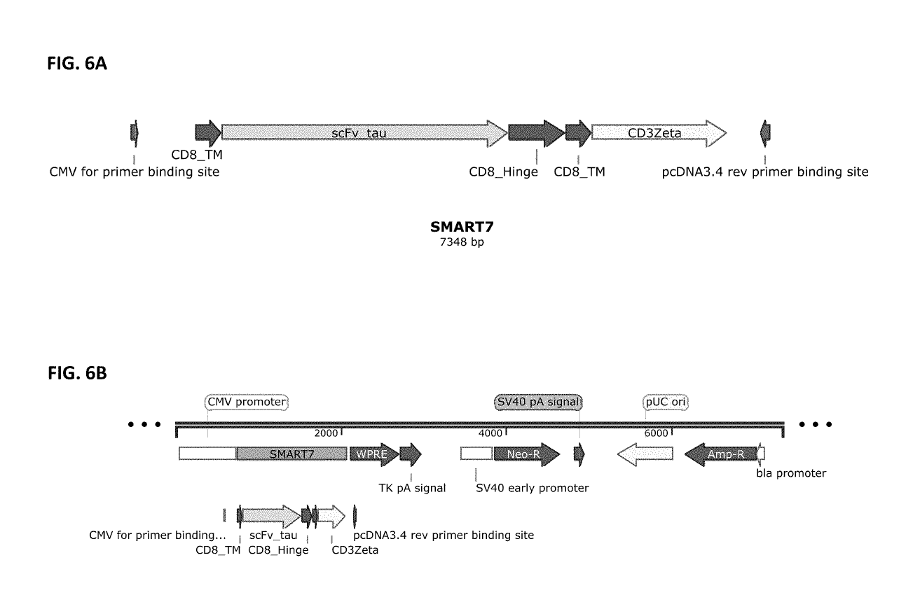

SMART7 is composed of the elements CD8SS>>anti-Tau_NFT>>CD8 Hinge>>CD8 transmembrane>TCRzeta intracellular domain. SMART7 may lead to ITAM mediated survival and anti-inflammatory/pro-repair signaling in the context of Tau multimers or aggregates. The ITAM for CD3Z may be more potent than that of DAP12. The sequences for SMART7 are provided in SEQ ID NO: 27 (amino acid) and SEQ ID NO: 43 (polynucleotide), and the vectors are shown in FIG. 6. This construct may allow a selective and local activation of ITAM activity in the context of Tau soluble or insoluble multimers at sites of AD pathology.

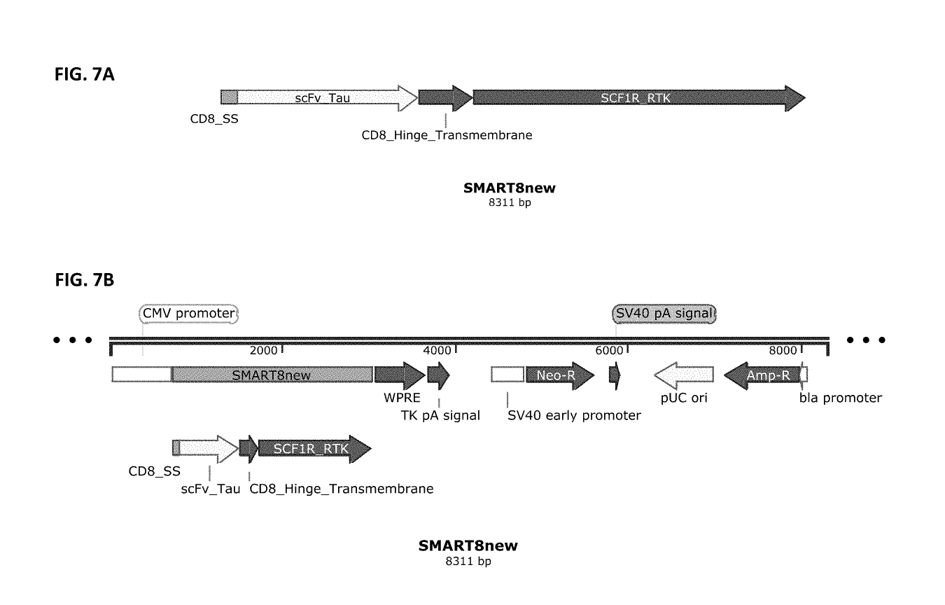

SMART8 is composed of the elements CD8SS>>anti-Tau_NFT>>CD8 Hinge>>CD8 transmembrane>CSF1R RTK intracellular domain. SMART8 may lead to RTK mediated survival and anti-inflammatory/pro-repair signaling in the context of Tau multimers or aggregates. The RTK of CSF1R may be highly potent as a pro-survival and anti-inflammatory signal. The sequences for SMART8 are provided in SEQ ID NO: 28 (amino acid) and SEQ ID NO: 44 (polynucleotide), and the vectors are shown in FIG. 7. This construct may allow a selective and local activation of ITAM activity in the context of Tau soluble or insoluble multimers at sites of AD pathology.

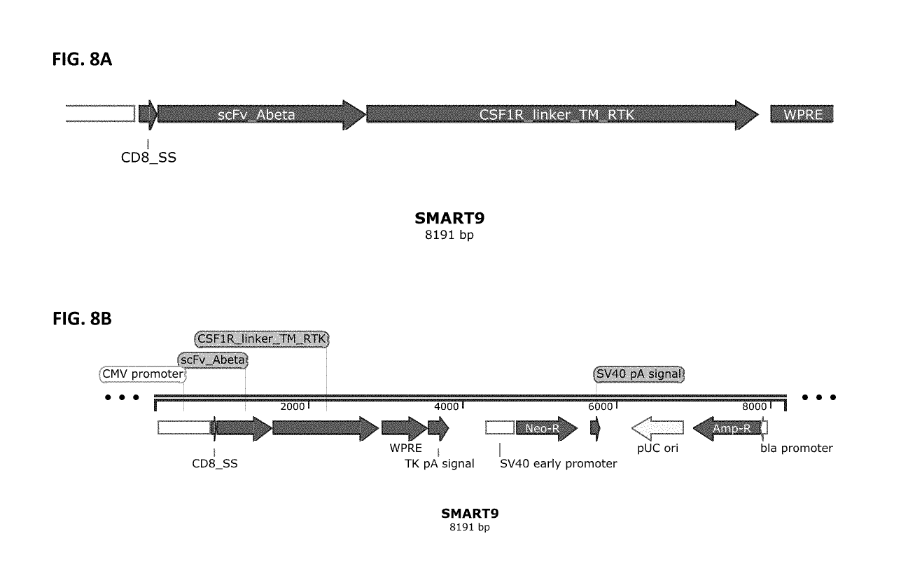

SMART9 is composed of the elements CD8SS>>anti-Abeta>>CSF1R linker, Transmembrane and RTK intracellular domain and may lead to RTK mediated survival and anti-inflammatory/pro-repair signaling in the context of Abeta multimers or aggregates. The sequences for for SMART9 are provided in SEQ ID NO: 29 (amino acid) and SEQ ID NO: 45 (polynucleotide), and the vectors are shown in FIG. 8. This construct may allow a selective and local activation of RTK activity in the context of Abeta soluble or insoluble multimers at sites of AD pathology. Compared to SMART3, this vector may provide a more native structure for the CSF1R receptor component.

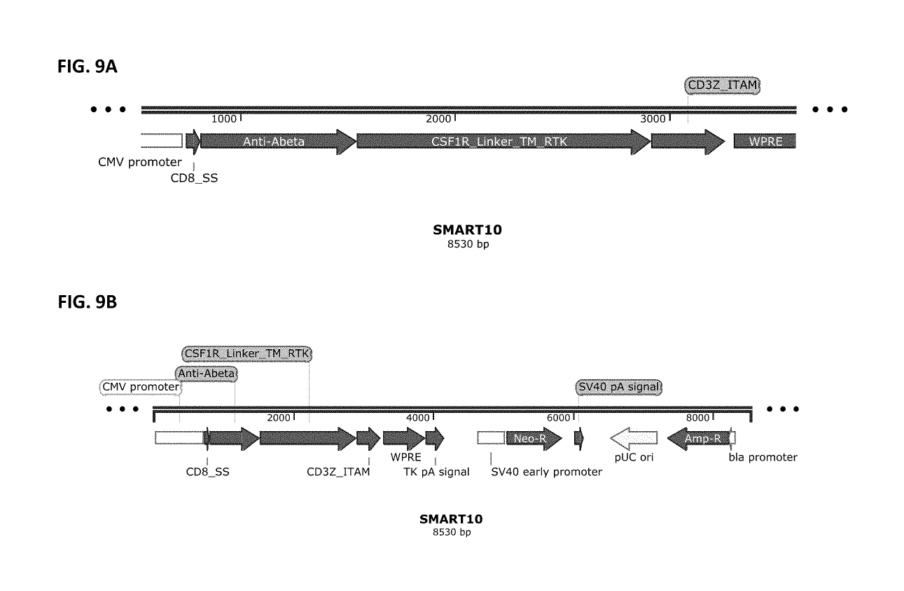

SMART10 is composed of the elements CD8SS>>anti-Abeta>>CSF1R Linker, Transmembrane, and RTK Intracellular domain>>CD3Zeta ITAM domain. SMART10 may lead to both RTK and ITAM mediated survival and anti-inflammatory/pro-repair signaling in the context of Abeta multimers or aggregates. The sequences for SMART10 are provided in SEQ ID NO: 30 (amino acid) and SEQ ID NO: 46 (polynucleotide), and the vectors are shown in FIG. 9. This construct may allow a selective and local activation of RTK activity in the context of Abeta soluble or insoluble multimers at sites of AD pathology. Compared to SMART9, this vector contains multiple signaling domains that activate an M2-like polarization for myeloid cells; such multiple cooperative domains may more robustly activate myeloid cells.

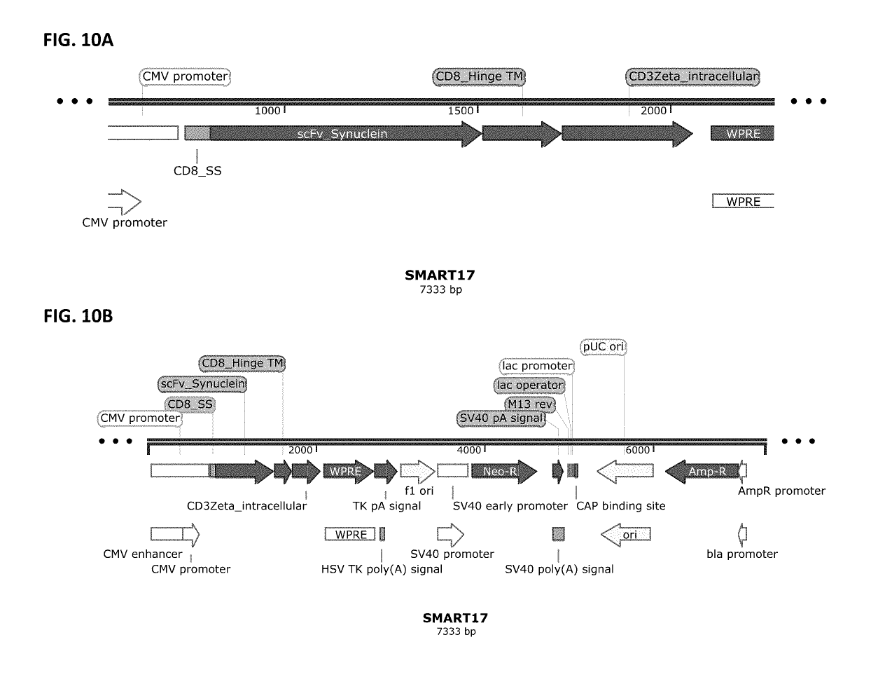

SMART17 is composed of the elements CD8SS>>anti-alphaSynuclein>>CD8Hinge>>CD8TM>>CD3Z ITAM. SMART17 may lead to ITAM mediated survival and anti-inflammatory/pro-repair signaling in the context of aSynuclein multimers or aggregates, as is typified in Parkinson's disease, Lewy body dementia, and related disorders and animal models. The sequences for SMART17 are provided in SEQ ID NO: 31 (amino acid) and SEQ ID NO: 47 (polynucleotide) and the vectors are shown in FIG. 10.

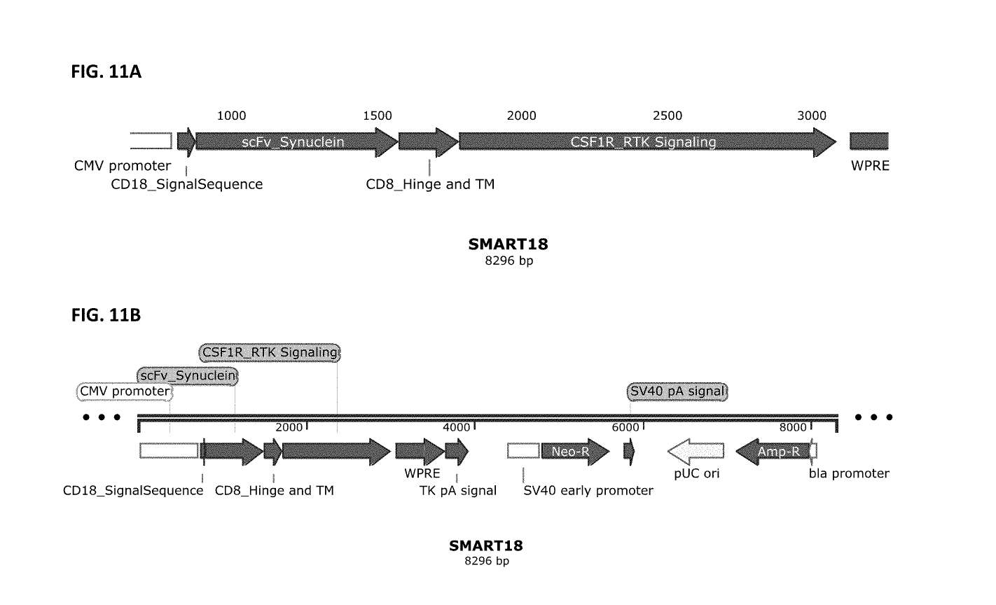

SMART18 is composed of the elements CD8SS>>anti-aSynuclein>>CD8 Hinge>>CD8TM>>CSF1R RTK intracellular domain. SMART18 may lead to RTK mediated survival and anti-inflammatory/pro-repair signaling in the context of Abeta multimers or aggregates. The sequences for SMART18 are provided in SEQ ID NO: 32 (amino acid) and SEQ ID NO: 48 (polynucleotide), and the vectors are shown in FIG. 11. This construct may allow a selective and local activation of RTK activity in the context of Abeta soluble or insoluble multimers at sites of AD pathology.

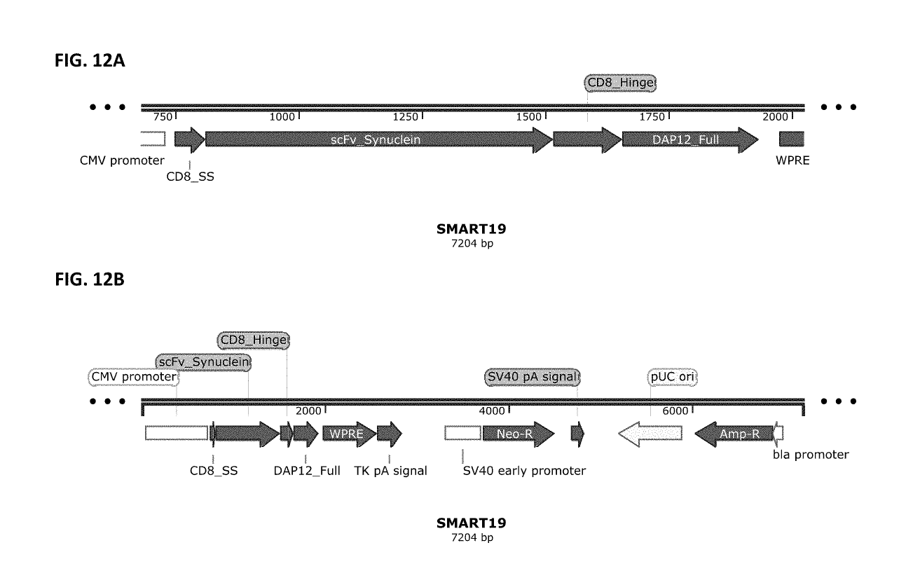

SMART19 is composed of the elements CD8SS>>anti-AlphaSynuclein>>CD8 Hinge>>DAP12 transmembrane and full length intracellular domains. SMART19 may lead to ITAM mediated survival and anti-inflammatory/pro-repair signaling in the context of alpha-Synuclein multimers or aggregates. The ITAM for DAP12 is normally expressed in myeloid cells, unlike CD3Z, and thus may lead to a more selective activation in these cells. The sequences for SMART19 are provided in SEQ ID NO: 33 (amino acid) and SEQ ID NO: 49 (polynucleotide), and the vectors are shown in FIG. 12. This construct may allow a selective and local activation of ITAM activity in the context of aSynuclein soluble or insoluble multimers at sites of PD or LBD pathology.

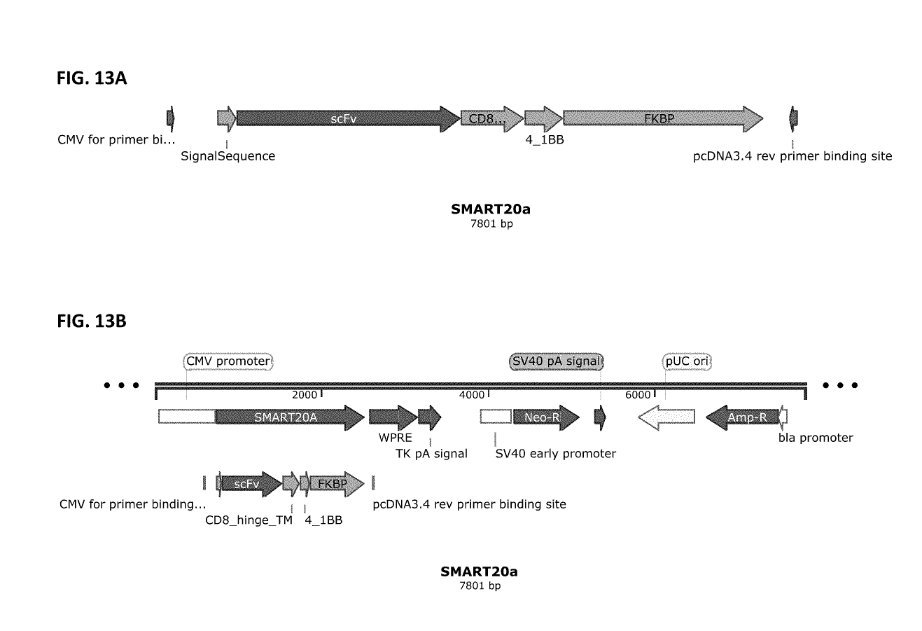

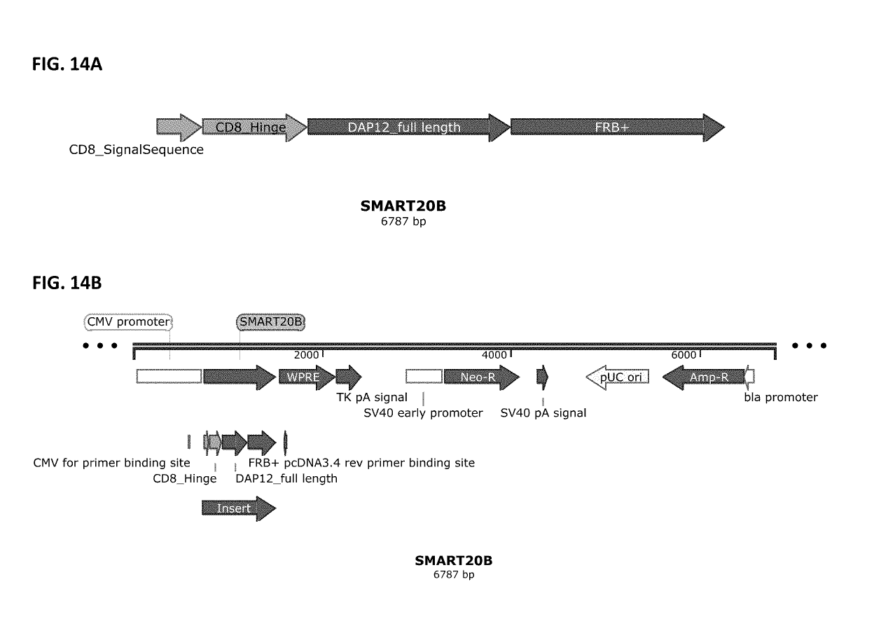

SMART20 is a two-component SMART system composed of component A and component B. Component A is composed of a CD8 signal sequence, anti-Abeta scFv, a CD8 hinge and CD8 transmembrane domain, and a 4-1BB signaling domain upstream of a FKBP inducible dimerization domain. Binding to multimerized or aggregated Abeta can lead to dimerization and multimerization of the receptor components including the dimerization domain. However, this construct lacks an active ITAM or an active kinase domain, and thus may not induce myeloid cell functions when transduced into myeloid cells. The second fragment, component B, is composed of a CD8 signal sequence and hinge domain, a full length DAP12 domain, and a dimerization component FRB+. In the presence of rapsyn, rapamycin, or a similar molecule, components A and B can dimerize, leading to induction of signaling through the SMART receptor. Local application of rapsyn, rapsyn, such as ICV administration, can localize the induction of myeloid cells in the presence of Abeta aggregates or multimers. Co-transduction of the A and B components of SMART20 can be achieved with 2 lentiviral vectors, by transfection and selection using 2 selectable markers such as puromycin and G418, or as described previously for the 1-component SMART receptors. The sequences for SMART20A are provided in SEQ ID NO: 34 (amino acid) and SEQ ID NO: 50 (polynucleotide), and the sequences for SMART20B are provided in SEQ ID NO: 35 (amino acid) and SEQ ID NO: 51 (polynucleotide). The vectors for SMART 20 components A and B are shown in FIG. 13 and FIG. 14, respectively.

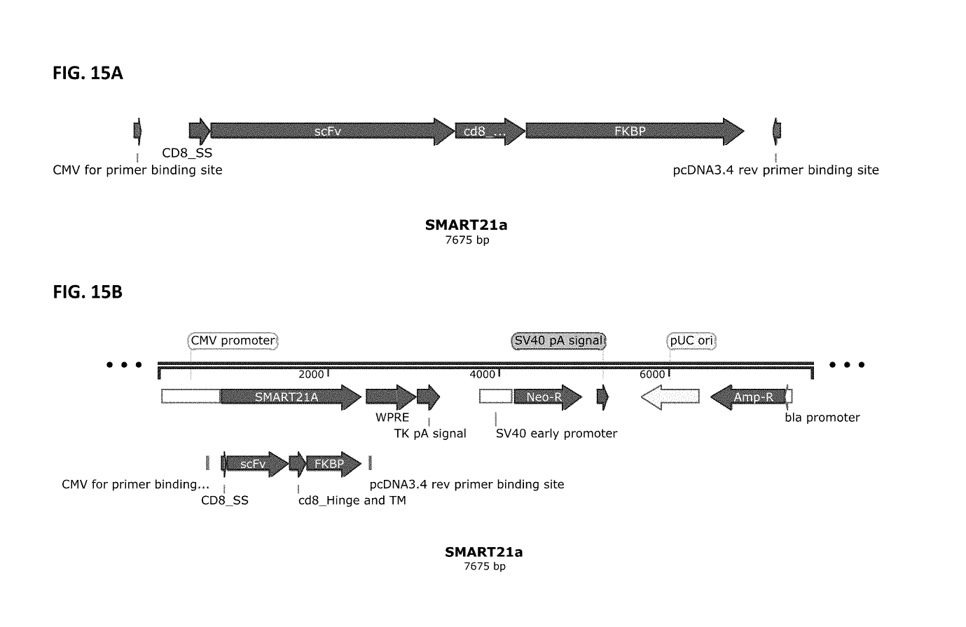

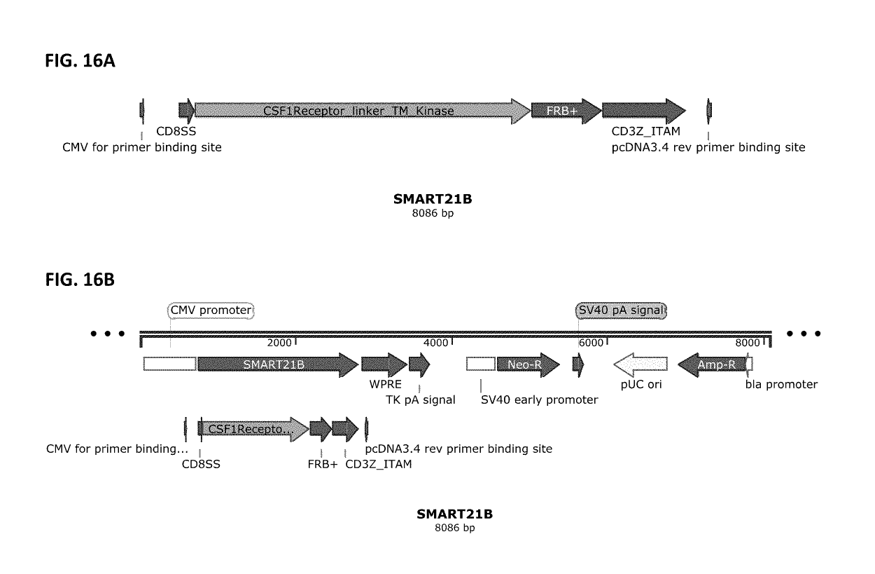

SMART21 is a two-component SMART vector, with component A being composed of the elements CD8SS>>>>anti-Abeta>>CD8 Hinge>>CD8TM>>FKBP and component B being composed of the elements CD8SS>>CSF1R linker, transmembrane, and RTK intracellular domain>>FRB+>>CD3Zeta ITAM domain. In the presence of rapsyn or rapamycin and upon recognition of Abeta multimers or aggregates, myeloid cells expressing components A and B can induce ITAM and RTK signaling. In this context, the cells may expand, survive, and show a pro-repair phenotype as described for the 1-component receptors that harbor similar domains. The sequences for SMART21A are provided in SEQ ID NO: 36 (amino acid) and SEQ ID NO: 52 (polynucleotide), and the sequences for SMART21B are provided in SEQ ID NO: 37 (amino acid) and SEQ ID NO: 53 (polynucleotide). The vectors for SMART 21 components A and B are shown in FIG. 15 and FIG. 16, respectively.

### Example 2: Transduction of SMART Vectors into Myeloid Cells In Vitro

SMART vectors can be transduced into primary human myeloid cells or animal model myeloid cells by transfection or transduction using a viral vector such as a lentivirus vector. To determine whether the SMART vectors described in Example 1 can be expressed as intact chimeric proteins, myeloid cells are transfected with individual SMART plasmid vectors. Linearized plasmid vectors are electroporated under optimized conditions and stable transfectants are selected by addition of G418, hygromycin, or another selectable marker to cell cultures, as appropriate for the vector used.

Western blot or FACS analyses of myeloid cells with antibodies directed against the different chimeric receptor domains are used to confirm expression of the chimeric receptors in the cells. Whole cell lysates from mock transfectants (cells containing the vector without a SMART insert) and from myeloid cells transfected with SMART vectors are compared. For example, Western blot of whole cell lysates from cells transfected with a SMART vector that includes the CD3zeta domain with an anti-CD3zeta antibody probe can show expression of the intact chimeric receptor protein in cells transfected with the chimeric receptor but not in the mock transfectants. Flow cytometric analysis with anti-human Fab specific antibodies can further confirm the cell-surface expression of the SMART chimeric receptors on cell transfectants. Similarly, SMART vectors are inserted into lentiviral vectors which can then be produced and used to transduce the SMART construct into myeloid cells.

### Example 3: Abeta-Specific Repair Functions of Myeloid Cells Expressing SMART2, SMART3, SMART4, or SMART5 Chimeric Receptors

The function of the SMART2, SMART3, SMART4, or SMART5 chimeric immunoreceptors in myeloid cells is assessed by expressing these constructs individually or in combination in primary human myeloid cells or in cell lines such as THP1. Cells are grown and expanded in standard in vitro cell culture media conditions in the presence or absence of ligands of the chimeric receptor, which for SMART2, SMART3, SMART4, and SMART5 chimeric receptors is Abeta. Abeta is included as a monomer or as multimers generated by the formation of soluble or insoluble multimers of Abeta. Engagement and multimerization of SMART receptors can lead to signaling and improved myeloid cell survival and division. Such multimerization may occur in the context of multimerized targets due to intrinsic properties of the Abeta ligand, or in the context of crosslinking agents such as secondary antibodies.

Expression of SMART receptors on the surface of myeloid cells is confirmed by FACS analysis using antibodies specific for extracellular domains of the chimeric receptors, such as human variable domains or CD8 hinge domains. FACS or magnetic bead based methods can be further used to enrich cells with higher or lower levels of the receptors.

Clones or non-clonal cells that express functional SMART chimeric receptors may induce induce signaling events which are assayed using standard methods. For example, SMART2, SMART3, SMART4, and SMART5 expressing cells are assayed for induction of SYK kinase phosphorylation by immunopurification or immunoprecipitation of SYK, followed by Western blotting the total protein lysate with an antibody to phosphotyrosine. Cells are also assayed for induction of signaling and downstream events by measuring their ability to secrete cytokines specifically upon co-culture with multimeric ligands. For example, in the case of SMART2, SMART3, SMART4, and SMART5, multimerized or aggregated forms of Abeta are used to culture cells. Chimeric myeloid clones which express the desired chimeric receptors are isolated.

### Example 4: Transduction of Bone Marrow-Derived Dendritic Cells (BMDC) with SMART Receptors

Lentiviral mediated transduction of myeloid cells with vectors encoding SMART2, SMART3, SMART4, or SMART5 chimeric receptors is performed. Monocytes are isolated from buffy coats of healthy donors following Lymphoprep gradient centrifugation and positive or negative magnetic antibody separation kit (Miltenyi Biotec, Leiden, Netherlands). Purity is assessed by flow cytometry of anti-CD14-PE stained cells. Isolated cells are cultured in 24-well plates at 250,000 cells/well in 0.5 mL of RPMI medium (RPMI 1640, Life Technologies, Carlsbad, Calif.) supplemented with 2 mM L-glutamine (Life Technologies), 2.5% (vol/vol) heat inactivated fetal calf serum (FCS, Hyclone Perbio, Thermo Scientific, Rockford, Ill.), 100 U/mL penicillin, 100 μg/mL streptomycin (Life Technologies), IL-4 at 500 IU/mL, and GM-CSF at 1,000 IU/mL (Gentaur, Kampenhout, Belgium) at 37° C. in a humidified atmosphere containing 5% (vol/vol) CO₂. To assess the impact of fetal calf serum on MDDC transduction efficiency, sera from Biochrom (Merc Milipore, Overijse, Belgium), Bovogen Biologicals (East Keilor, Australia), Lonza (Verviers, Belgium) and PAA (GE Healthcare, Diegem, Belgium) are also used. To ensure standardized transduction, lentiviral supernatants are titrated. Six days post-transduction the cells are plated in a 96-well plate at 50,000 cells/well and infected (50 ng p24) by spinoculation (90 min, 950 g, 32° C.), with continuous spinning in a centrifuge, in presence of 1 μM ritonavir (NIH AIDS Reagent Program, Germantown, Md.) in a final volume of 200 μL. On day 1 post-infection, medium is refreshed. Infection is measured on day 3 by flow cytometry, gating on live cells as determined by propidium iodide staining (Miltenyi Biotec).

Viral reverse transcriptase (RT) activity, quantitative real-time qPCR for viral DNA of long terminal repeat sequences, or ELISA of p24 viral protein are performed using standard techniques. Supernatant of lentiviral vector encoding a scrambled sequence or an eGFP marker gene showed an MOI of 10 when measured on 293T cells and provided over 95% MDDC transduction efficiency. This lentiviral supernatant expressed RT activity of 5,550 mU/ml (equivalent of 1 μg of p24/ml as assessed by ELISA) in previous studies. Aliquots of this supernatant are included in all subsequent reverse transcriptase activity assays to serve as a standard reference for viral production.

Monocytes are obtained by positive magnetic bead-based selection of CD14+ cells. On day 1 post-monocyte isolation, medium is replaced with fresh medium containing 50% lentiviral supernatant. RT activity of 2,750-5,550 mU/ml is used. Cells are subsequently spinoculated (90 min, 950 g, 32° C.) in the presence of polybrene (4 μg/mL; Sigma-Aldrich, Diegem, Belgium). Medium is refreshed 24 h post-transduction and cells are cultured in the presence of IL-4 and GM-CSF until day 6. In some experiments, maturation is induced with LPS (100 ng/mL; Sigma-Aldrich). From day 6 post-transduction onwards, cells are cultured in 10% FCS (vol/vol) RPMI medium supplemented with glutamine, penicillin and streptomycin.

SMART vector sequences are inserted into a pLKO.1 based vectors expression cassette under the direction of an appropriate promoter element. Transfection of vectors into 293T cells is performed using standard approaches. Viral production is achieved using standard second or third-generation lentiviral transduction vector packaging production kits, such as Virapower (Life Technologies/Fisher Scientific) using the manufacturer's instructions. The titer of the viral supernatants is measured by quantification of reverse transcriptase activity via real time-PCR and expressed as equivalent p24 as described above.

Although lentiviral vectors can be inhibited in human myeloid cells, Witkowski et al. (Witkowski, Vermeire et al., PLoS One, e0133651, 2015) optimized the transduction of MDDCs by investigating the effect of a range of parameters, including additives such as polybrene, spinoculation, and experimental timeline. This optimized protocol is subsequently used in the experiments described herein. Transduction is performed by spinoculation as described above in the presence of polybrene, which can facilitate virus-cell binding and entry. To measure transduction efficiency, a pLKO.1-derived lentiviral vector encoding an eGFP marker gene is used. Transduction efficiency, as well as the MDDC phenotype, is evaluated five days post-transduction.

### Example 5: Normalization and Reduction of Toll-Like Receptor (TLR) Responses in Dendritic Cells Expressing SMART Receptors SMART2, SMART3, SMART4, or SMART5 in the Presence of Ligand

Bone marrow-derived dendritic cells (BMDC), expressing control vector or SMART encoding vectors, are introduced by viral transduction as described in Example 4 above or by electroporation. BMDCs are subsequently stimulated by culturing with TLR ligands, such as LPS, CpG DNA, and zymosan, for 16 h. Conditioned media is collected and ELISA assays are performed in order to evaluate secretion of the cytokines IFN-α, IFN-b, IL-6, IL-12 p70, and TNF. BMDC cells expressing pro-repair SMART vectors such as the SMART2-SMART5, that signal through RTK or ITAM domain receptors, may secrete significantly more IL-12, p70, and TNF upon simulation with multimerized ligand than control BMDC cells. For SMART2-SMART5 vectors, for instance, such ligands would be represented by Abeta multimers or aggregates of Abeta. It is further believed that the presence of ligands of pro-repair SMART receptors will reduce the expression levels of IL-12, p70, and TNF.

### Example 6: Ability of Pro-Repair SMART Receptor-Expressing BMDC to Mediate Normalization and Reduction of Antigen-Specific T-Cell Proliferation in the Presence of Ligand

Bone marrow-derived dendritic cells (BMDC) that express pro-repair SMART vectors such as SMART2-SMART5 may, in the presence of multimeric ligand, inhibit antigen-specific T-cell proliferation. For example, the Ovalbumin (OVA)-specific T-cell response induced by BMDCs can be determined by CFSE dilution. BMDCs are isolated by MACS after 6 days of culture and plated at 1×10⁴ cells per well in a round bottom 96 well plate with OVA (2 or 0.5 mg/mL) and CpG DNA (100 or 25 nM) in the presence of GM-CSF (10 ng/mL) for 4 h. CD4 T-cells from the spleen and lymph nodes of OT-II transgenic mice are isolated using the Dynal Mouse CD4 Negative Isolation Kit (Invitrogen) and stained with CFSE (final 0.8 mM). After 4 h of DC culture, 1×10⁵ CFSE-labeled CD4 OT-II T-cells are added into each well and incubated for 72 h. After culturing, cells are stained with an anti-CD4 monoclonal antibody and flow cytometry is performed to detect CFSE dilution of gated CD4 OT-II T-cells. Data analysis to calculate the percentage of divided and division index is performed by Flowjo software (Treestar) (Eur. J. Immunol. 2012. 42: 176-185). The presence of ligand, such as mutimerized or aggregated Abeta at various concentrations, can suppress T cell proliferation relative to the absence of the multimerized ligand. Cytokine concentrations in the culture supernatants are determined using mouse IFN-α4, IFN-b, IL-6, IL-12 p70, TNF, and IL-10 ELISA kits (eBioscience) and VeriKine Mouse IFN-b ELISA kit (PBL interferon source) according to the manufacturer's protocol. Levels of mRNA for these cytokines are also measured by Quantitative RT-PCR (qRT-PCR). Total RNA prepared using the RNeasy plus mini kit (QIAGEN) is reverse-transcribed with Superscript III Reverse Transcriptase (Invitrogen) using oligo dT primer according to the manufacturer's protocol. Quantitative PCR is performed using the Power SYBR Green PCR Master Mix (Applied Biosystems) and 7900HT (Applied Biosystems) according to the manufacturer's protocol. The sequences of IFN-α4, IFN-b, IL-6, IL-12 p70, and TNF primers are described previously. (e.g., Hamerman, J A, Eur. J. Immunol. 2012. 42: 176-185).

### Example 7: BMDC Mediated Induction of Antigen-Specific T-Cell Proliferation when Expressing Pro-Inflammatory SMART Receptors in the Presence of Ligand

Bone marrow-derived dendritic cells (BMDC) that express pro-inflammatory SMART vectors may, when exposed to multimeric ligands such as Abeta oligomers or plaques, Tau fibrils, or A synuclein aggregates, induce antigen-specific T-cell proliferation. For example, the Ovalbumin (OVA)-specific T-cell response induced by BMDCs can be determined by CFSE dilution. BMDCs are isolated by MACS after 4-6 days of culture and plated at 1×10⁴ cells per well in a round bottom 96 well plate with OVA (2 or 0.5 mg/mL) and CpG DNA (100 or 25 nM) in the presence of GM-CSF (10 ng/mL) for 4 h. CD4 T-cells from the spleen and lymph nodes of OT-II transgenic mice are isolated using the Dynal Mouse CD4 Negative Isolation Kit (Invitrogen) and stained with CFSE (final 0.8 mM). After 4 h of DC culture, 1×10⁵ CFSE-labeled CD4 OT-II T-cells are added into each well and incubated for 72 h. After culturing, cells are stained with an anti-CD4 monoclonal antibody and flow cytometry is performed to detect CFSE dilution of gated CD4 OT-II T-cells. Data analysis to calculate the percentage of divided and division index is performed by Flowjo software (Treestar) (Eur. J. Immunol. 2012. 42: 176-185).

Cytokine concentrations in the culture supernatants are determined using mouse IFN-α4, IFN-b, IL-6, IL-12 p70, TNF, and IL-10 ELISA kits (eBioscience) and VeriKine Mouse IFN-b ELISA kit (PBL interferon source) according to the manufacturer's protocol. Levels of mRNA for these cytokines are also measured by Quantitative RT-PCR (qRT-PCR). Total RNA prepared using the RNeasy plus mini kit (QIAGEN) is reverse-transcribed with Superscript III Reverse Transcriptase (Invitrogen) using oligo dT primer according to the manufacturer's protocol. Quantitative PCR is performed using the Power SYBR Green PCR Master Mix (Applied Biosystems) and 7900HT (Applied Biosystems) according to the manufacturer's protocol. The sequences of IFN-α4, IFN-b, IL-6, IL-12 p70, and TNF primers are described previously. (e.g., Hamerman, J A, Eur. J. Immunol. 2012. 42: 176-185). In the presence of multimerized ligand for these SMART receptors, T cell proliferation may be induced.

To stimulate cells with SMART receptor ligands, ligands are presented, either in an immobilized plate-bound format that assures multimerization, or in solution, at a concentration range of 0.04-125 ug/ml or more, to BMDC cells that have been matured using standard methods. Cells are cultured in normal conditions, with or without MCSF, or are deprived of MCSF for 48 hrs. 10⁴ to 10⁶ cells are seeded onto the culture plate in the presence or absence of the monomeric or multimerized SMART ligand for a period of 2 to 48 hours. Subsequently, cells are scarped or otherwise isolated and used in a T cell proliferation assay in the presence of the appropriate T cell ligand, such as OVA in the context of OVA-specific T cells.

### Example 8: Normalization and Reduction of Toll-Like Receptor (TLR) Responses in Macrophages by Pro-Repair SMART Vectors in the Presence of Ligand

Signaling through SMART receptors (e.g. through local activation of ITAM or RTK or other such signaling pathways) in the context of multimerized or aggregated ligand, or a high local ligand concentration, may reduce and normalize TLR responses in macrophages.

To elicit primary macrophages, mice are treated with 1.5 ml of 2% thioglycollate medium by intraperitoneal injection, and cells are then isolated by peritoneal lavage. To generate BMDM, total bone marrow is cultured in DMEM supplemented with 10% bovine calf serum, 5% horse serum, and 6 ng/ml recombinant human CSF-1 (R&D Systems). Cells are cultured for 5-6 days, and adherent cells are detached with 1 m MEDTA in PBS. Cells are stained with commercially available antibodies: anti-CD11b, anti-CD40, anti-GR1 (BD Pharmingen), and F4/80 (Caltag Laboratories).

BMDM are re-plated and allowed to adhere for 4 to 48 hrs at 37° C., in the absence or presence of MCSF, and subsequently incubated in the presence or absence of multimeric SMART ligands such as Abeta oligomers or plaques, Tau fibrils, or A synuclein aggregates at a concentration of 0.04-125 ug/ml or more. TLR agonists, such as LPS (Salmonella abortus equi), zymosan (Saccharomyces cerevisiae), and CpG 1826 DNA (purchased from e.g., Sigma-Aldrich), or vehicle only, are subsequently added. Ligands may be presented in solution or are adhered to the plastic surface using standard techniques. Cell culture supernatant is collected 24 h after stimulation and the the levels of IFN-α4, IFN-b, IL-6, IL-12 p70, and TNF cytokines are measured by ELISA or by cytometric bead array (BD Biosciences mouse inflammation kit).

### Example 9: Induction of the Anti-Inflammatory Cytokine IL-10 in Bone Marrow-Derived Myeloid Precursor Cells by Pro-Repair SMART Vectors in the Presence of Ligand

Bone marrow-derived myeloid precursor cells expressing pro-repair SMART receptors may show an increase in the anti-inflammatory cytokine IL-10 in the context of multimerized or aggregated ligand forms, a high local concentration of ligand, stimulation with 100 ng/ml LPS (Sigma), and co-culturing with apoptotic cells.

Isolation of bone marrow-derived myeloid precursor cells is performed as follows. Bone marrow cells are isolated from adult 6-8 week-old female C57BL/6 mice (Charles River, Sulzfeld, Germany) from the medullary cavities of the tibia and femur of the hind limbs. Removal of erythrocytes is performed by lysis with a hypotonic solution. Cells are cultured in DMEM medium (Invitrogen) containing 10% fetal calf serum (Pan Biotech) and 10 ng/ml of GM-CSF (R&D Systems) in 75 cm² culture flasks (Greiner Bio-One). After 24 h, non-adherent cells are collected and re-seeded in fresh 75 cm² culture flasks. Medium is changed after 5 d and cells are cultured for an additional 10-11 d. The remaining cells are bone marrow-derived myeloid precursor cells, and are transduced with SMART vectors. The transduced cells are cultured in the absence or presence of MCSF for 4 to 48 hrs, and subsequently incubated in the presence or absence of multimeric SMART ligands such as Abeta oligomers or plaques, Tau fibrils, or A synuclein aggregates at a concentration of 0.04-125 ug/ml or more. TLR agonists, such as LPS (Salmonella abortus equi), zymosan (Saccharomyces cerevisiae), and CpG 1826 DNA (purchased from e.g., Sigma-Aldrich) or vehicle only control, are subsequently added. Ligands may be presented in solution or are adhered to the plastic surface using standard techniques. The level of IL-10 is examined in conditioned media in both the presence and absence of receptor ligand, LPS, or apoptotic cells. Supernatant is collected after 24 h, and the level of IL-10 released from the cells is determined by IL-10 ELISA according to manufacturer's instructions (QuantikineM mouse IL-10, R&D Systems) (JEM (2005), 201; 647-657; and PLoS Medicine (2004), 4|Issue 4|e124).

### Example 10: Induction of Phagocytosis of Apoptotic Neurons, Nerve Tissue Debris, Non-Nerve Tissue Debris, Bacteria, Other Foreign Bodies, and Disease-Causing Proteins in Cells from the Myeloid Lineage that Express Pro-Repair SMART Vectors in the Context of Multimerized or Aggregated Forms of Ligand

Expression of pro-repair SMART vectors in the context of multimerized or aggregated ligand forms may induce phagocytosis of apoptotic neurons, nerve tissue debris, non-nerve tissue debris, bacteria, other foreign bodies, and disease-causing proteins (e.g. A beta peptide, alpha synuclain protein, Tau protein, TDP-43 protein, prion protein, and huntingtin protein) by myeloid cells such as monocytes and microglia.

Monocytes are isolated from peripheral blood collected from adult C57BL/6 mice. Hypotonic lysis buffer is used to deplete erythrocytes. Cells are plated on culture dishes in RPMI medium (Invitrogen) containing 10% fetal calf serum (Pan Biotech). Cells are cultured for several hours at 37° C. in 10% CO₂. After trypsinization, adherent cells are collected and used for phagocytosis experiments.

Microglial cells are prepared from the brains of post-natal day 3 to 5 (P3 to P5) C57BL/6 mice. IMeninges are removed mechanically, and the cells are dissociated by trituration and cultured in basal medium (BME; GIBCO BRL) supplemented with 10% FCS (PAN Biotech GmbH), 1% glucose (Sigma-Aldrich), 1% L-glutamine (GIBCO BRL), and 1% penicillin/streptomycin (GIBCO BRL), for 14 d to form a confluent glial monolayer. To collect microglial cells, the cultures are shaken on a rotary shaker (200 rpm) for 2 h. The attached astrocytes are used for immunohistochemistry. The detached microglial cells are seeded in normal culture dishes for 1 h, and then all non-adherent cells are removed and discarded. Purity of the isolated microglial cells is about 95% as determined by flow cytometry analysis with antibody directed against CD11b (BD Biosciences). Microglial cells are cultured in basal medium.

Oligodendrocytes or neuron-enriched cells are prepared from the brain of C57BL/6 mouse embryos (E15-16). Brain tissue is isolated, mechanically dispersed, and seeded in culture dishes pre-coated with 0.01 mg/ml poly-L-ornithin (Sigma-Aldrich) and 10 μg/ml laminin (Sigma-Aldrich). Cells are cultured in neuronal condition medium (BME; GIBCO BRL) supplemented with 2% B-27 supplement (GIBCO BRL), 1% glucose (Sigma-Aldrich), and 1% FCS (PAN Biotech GmbH). Cells are cultured for 5-10 d to obtain morphologically mature oligodendrocytes.

To conduct phagocytosis assays of apoptotic neurons, nerve tissue debris, non-nerve tissue debris, bacteria, other foreign bodies, and disease-causing proteins, microglia, macrophage, dendritic cells, or other myeloid cells are transduced with SMART vectors. After transduction, myeloid cells are cultured for 72 h. Neurons are cultured for 5-10 d, and okadaic acid is then added at the final concentration of 30 nM for 3 h to induce apoptosis. Neuronal cell membranes are labeled with CellTracker CM-DiI membrane dye (Molecular Probes). After incubation, apoptotic neurons or other targets of phagocytosis are washed two times and added to the transduced microglial culture at an effector/target ratio of 1:20. At 1 and 24 h after addition of apoptotic neurons, the number of microglia having phagocytosed neuronal cell membranes is counted under a confocal fluorescence microscope (Leica). Apoptotic cells are counted in three different areas at a magnification of 60. The amount of phagocytosis is confirmed by flow cytometry. Moreover, 24, 48, or 72 h after the addition of apoptotic neurons, cells are collected and used for RT-PCR of cytokines.

To conduct microsphere bead or bacterial phagocytosis assays, myeloid cells such as microglia, macrophages, or dendritic cells are transduced with a SMART expression vector or a GFP control vector. Cells are then treated with multimerized or aggregated ligand or with no ligand. For instance, in the context of SMART vectors SMART2, SMART3, SMART4, or SMART5, the ligand is multimerized or aggregated Abeta at concentrations determined by serial titration. After 24 h, 1.00 μm of red fluorescent microsphere beads (Fluoresbrite Polychromatic Red Mi-crospheres; Polysciences Inc.) or fluorescent labeled bacteria are added for 1 h. Phagocytosis of microsphere beads or fluorescent labeled bacteria by microglia is analyzed by fluorescence microscopy. Furthermore, microglia are collected from the culture plates and analyzed by flow cytometry. The percentage of microglia having phagocytosed beads is determined. Because phagocytosis varies from one experiment to the other, the relative change in phagocytosis is also determined. Data are analyzed as the relative change in phagocytosis between SMART transduced microglia cultured in the presence or absence of multimerized ligand.

To conduct RT-PCR for analysis of inflammatory gene transcripts, myeloid cells are transduced with a SMART vector or control vector. Cells are then cultured on dishes and treated with multimerized or aggregated ligand or control. After 24, 48, and 72 h, RNA is isolated from microglia using an RNeasy Mini Kit (QIAGEN).

Reverse transcription of RNA is then performed. Quantitative RT-PCR by SYBR Green is performed on an ABI Prism 5700 Sequence Detection System (PerkinElmer). Amplification of GAPDH is used for sample normalization. The amplification protocol follows the GeneAmp 5700 Sequence Detection System Software (version 1.3). For detection of mouse GAPDH, TNF-alpha, IL-1, NOS2, and TGF-beta transcripts, the following forward and reverse primers are used at final concentrations of 200 nM (a similar approach can be used using human-specific primers for analysis of human cells):

GAPDH forward primer:
(SEQ ID NO: 54)
5′-CTCCACTCACGGCAAATTCAA-3′,
and
GAPDH reverse primer:
(SEQ ID NO: 55)
5′-GATGACAAGCTTCCCATTCTCG-3′;
TNF-α forward primer:
(SEQ ID NO: 56)
5′-CCGTCAGCCGATTTGCTATCT-3′,
and
TNF-α reverse primer:
(SEQ ID NO: 57)
5′-ACGGCAGAGAGGAGGTTGACTT-3′;
IL-1α forward primer:
(SEQ ID NO: 58)
5′-ACAA-CAAAAAAGCCTCGTGCTG-3′,
and
IL-1α reverse primer:
(SEQ ID NO: 59)
5′-CCATTGAGGTGGAGAGCTTTCA-3′;
NOS2 forward primer:
(SEQ ID NO: 60)
5′-GGCAAACCCAAGGTCTACGTTC-3′,
NOS2 reverse primer:
(SEQ ID NO: 61)
5′-TACCTCATTGGCCAGCTGCTT-3′;
and
TGF-β1 forward primer:
(SEQ ID NO: 62)
5′-AGGACCTGGGTTGGAAGTGG-3′,
and
TGF-β1 reverse primer:
(SEQ ID NO: 63)
5′-AGTTGGCATGGTAGCCCTTG-3′.

To conduct amyloid phagocytosis assays, HiLyteFluor™ 647 (Anaspec)-Abeta-(1-40) is resuspended in Tris/EDTA (pH 8.2) at 20 mM and then incubated in the dark for 3 d at 37° C. to promote aggregation. Myeloid cells transduced with SMART vectors (e.g. SMART2-SMART5) are pretreated for 24 h in low serum (0.5% FBS supplemented with insulin) with LPS (50 ng/ml), IFNγ (100 units/ml), and Abeta multimers, Abeta aggregates, or other SMART ligands. Cells are then subjected to extensive washing and aggregated fluorescently labeled Abeta peptide is added. Amyloid phagocytosis and surface expression of SMART receptors are determined by flow cytometric analysis 5 h post-addition of 100 nM aggregated HiLyteFluor™ 647-Ab-(1-40) (ASN NEURO (2010) 2(3): 157-170). Phagocytosis of other disease-causing proteins is conducted in a similar manner.

### Example 11: Increased Toll-Like Receptor (TLR) Responses in Macrophages Expressing Pro-Inflammatory SMART Vectors in the Presence of Ligand

Signaling through pro-inflammatory SMART receptors (e.g. through local activation of TLR5, 1-4BB, CD28, or CD3Zeta) in the context of multimerized or aggregated ligand or high local ligand concentration, may locally enhance TLR responses in macrophages or mimic such responses in the absence of TLR ligands.

To elicit primary macrophages, mice are treated with 1.5 ml of 2% thioglycollate medium by intraperitoneal injection, and cells are then isolated by peritoneal lavage. To generate BMDM, total bone marrow is cultured in DMEM supplemented with 10% bovine calf serum, 5% horse serum, and 6 ng/ml recombinant human CSF-1 (R&D Systems). Cells are cultured for 5-6 days, and adherent cells are detached with 1 mM EDTA in PBS. Cells are stained with commercially available antibodies, including anti-CD11b, anti-CD40, anti-GR1 (BD Pharmingen), and F4/80 (Caltag Laboratories).

BMDM are re-plated and allowed to adhere for 4 h at 37° C., and then TLR agonists, such as LPS (Salmonella abortus equi), zymosan (Saccharomyces cerevisiae), and CpG 1826 DNA (purchased from e.g., Sigma-Aldrich) are added.

Next, Lentiviral mediated transduction of the cells with vectors encoding SMART2, SMART3, SMART4, SMART5, or other chimeric receptors is performed. Monocytes are isolated from buffy coats of healthy donors following Lymphoprep gradient centrifugation and positive or negative magnetic antibody separation (Miltenyi Biotec, Leiden, Netherlands). Purity is assessed by flow cytometry of anti-CD14-PE stained cells. Isolated cells are cultured. Cells are matured to the appropriate phenotype. To ensure standardized transduction, lentiviral supernatants are titrated. Cells Cells are plated in a 96-well plate at 50,000 cells/well and infected (50 ng p24) by spinoculation (90 min, 950 g, 32° C.), with continuous spinning in a centrifuge, in presence of 1 μM ritonavir (NIH AIDS AIDS Reagent Program, Germantown, Md.) in a final volume of 200 μL. On day 1 post-infection, medium is refreshed. Infection is measured on day 3 by flow cytometry, gating on live cells as determined by propidium iodide staining (Miltenyi Biotec).

Viral reverse transcriptase (RT) activity, quantitative real-time qPCR for viral DNA of long terminal repeat sequences, or ELISA of p24 viral protein are performed using standard techniques. Supernatant of lentiviral vector encoding a scrambled sequence or an eGFP marker gene showed an MOI of 10 when measured on 293T cells and provided over 95% transduction efficiency. This lentiviral supernatant expressed RT activity of 5,550 mU/ml (equivalent of 1 μg of p24/ml as assessed by ELISA) in previous studies. Aliquots of this supernatant are included in all subsequent reverse transcriptase activity assays to serve as a standard reference for viral production.

On day 1 post isolation, medium is replaced with fresh medium containing 50% lentiviral supernatant. RT activity of 2,750-5,550 mU/ml is used. Cells are subsequently spinoculated (90 min, 950 g, 32° C.) in the presence of polybrene (4 μg/mL; Sigma-Aldrich, Diegem, Belgium). Medium is refreshed 24 h post-transduction and cells are cultured using standard methods.

To generate virus, transfection of vectors into 293T cells is performed using standard approaches. Viral production is achieved using standard second or third-generation lentiviral transduction vector packaging production kits, such as Virapower (Life Technologies/Fisher Scientific) using the manufacturer's instructions. The titer of the viral supernatants is measured by quantification of reverse transcriptase activity via real time-PCR and expressed as equivalent p24.

Although lentiviral vectors can be inhibited in human myeloid cells, Witkowski et al. (Witkowski, Vermeire et al., PLoS One, e0133651, 2015) optimized the transduction of myeloid cells by investigating the effect of a range of parameters, including additives such as polybrene, spinoculation, and experimental timeline. This optimized protocol is subsequently used in the experiments described herein. Transduction is performed by spinoculation as described above in the presence of polybrene, which can facilitate virus-cell binding and entry. To measure transduction efficiency, a pLKO.1-derived lentiviral vector encoding an eGFP marker gene is used. Transduction efficiency, as well as the macrophage phenotype, is evaluated five days post-transduction.

Cells are exposed to multimeric SMART ligands such as Abeta oligomers or plaques, Tau Tau fibrils, or A synuclein aggregates, cell culture supernatant is collected 24 h after stimulation, and the levels of IFN-α4, IFN-b, IL-6, IL-12 p70, and TNF cytokines are measured by ELISA or by cytometric bead array (BD Biosciences mouse inflammation kit).

### Example 12: Inhibited Expression of Anti-Inflammatory Cytokine IL-10 in Bone Marrow-Derived Myeloid Precursor Cells Expressing Pro-Inflammatory SMART Vectors in the Presence of Ligand

Bone marrow-derived myeloid precursor cells expressing pro-inflammatory SMART receptors may show a decrease in the anti-inflammatory cytokine IL-10 in the context of multimerized or aggregated ligand, high local ligand concentration, stimulation with 100 ng/ml LPS (Sigma), and co-culturing with apoptotic cells.

Isolation of bone marrow-derived myeloid precursor cells is performed as follows. Bone marrow cells are isolated from adult 6-8 week-old female C57BL/6 mice (Charles River, Sulzfeld, Germany) from the medullary cavities of the tibia and femur of the hind limbs. Removal of erythrocytes is performed by lysis with a hypotonic solution. Cells are cultured in DMEM medium (Invitrogen) containing 10% fetal calf serum (Pan Biotech) and 10 ng/ml of GM-CSF (R&D Systems) in 75 cm² culture flasks (Greiner Bio-One). After 24 h, non-adherent cells are collected and re-seeded in fresh 75 cm² culture flasks. Medium is changed after 5 d and cells are cultured for an additional 10-11 d. The remaining cells are bone marrow-derived myeloid precursor cells, and are transduced with SMART pro-inflammatory receptors. The transduced cells are then examined for the level of IL-10 in conditioned media in both the presence and absence of an appropriate SMART receptor ligand, present at an appropriate concentration or ratio to the myeloid cells as determined by titration. Supernatant is collected after 24 h, and the level of IL-10 released from the cells is determined by IL-10 ELISA according to the manufacturer's instructions (QuantikineM mouse IL-10, R&D Systems) (JEM (2005), 201; 647-657; and PLoS Medicine (2004), 4|Issue 4|e124).

### Example 13: SMART Ligand-Mediated Induction of the Expression of CD83 and CD86 on Human Dendritic Cells (DCs) Expressing Pro-Repair SMART Receptors

The ability of pro-repair SMART receptors to inducibly modify expression of CD83 and CD86 is evaluated.

SMART vector transduced myeloid cells are generated as described above. On day 5 of monocyte differentiation to dendritic cells, immature human DCs are harvested and plated at 1 million cells per well and incubated at 37 C, 5% CO₂ in the presence of multimeric SMART ligands such as Abeta oligomers or plaques, Tau fibrils, or A synuclein aggregates and the absence of cytokine. FACS analysis of CD86, CD83, CD11c, HLA-DR, and LIN (BD Biosciences) is performed on a BD FACS Canto 48 hours later. Data analysis is performed with FlowJo (TreeStar) software version 10.0.7. Levels of CD83 and CD86 are evaluated on CD11c+HLA-DR+LIN− cell populations.

Alternatively, Day 5 immature human dendritic cells are plated at 100,000 cells per well in a U-bottom non-TC treated 96 well plate in media without cytokine, with or without LPS-removed anti-human secondary antibody (Jackson ImmunoResearch) at 20ug/ml. FACS analysis for CD86, CD83, CD11c, HLA-DR, and LIN (BD Biosciences) is performed 48 hrs post antibody addition.

The presence of multimerized or aggregated forms of SMART receptor ligand (e.g. aggregated or multimerized Abeta in the context of SMART2-SMART5) may increase the frequency of CD83+CD86+ DCs compared to the absence of such ligand.

### Example 14: SMART Receptor Ligand-Mediated Induction of Syk Phosphorylation in SMART Transduced Myeloid Cells

Spleen tyrosine kinase (Syk) is an intracellular signaling molecule that functions downstream of DAP12, CD3Zeta, and other ITAM signaling modules by phosphorylating several substrates, thereby facilitating the formation of a signaling complex leading to cellular activation and inflammatory processes. The ability of SMART receptor ligands to induce Syk activation in SMART transduced myeloid cells is determined by culturing transduced human or mouse macrophages or primary human dendritic cells and measuring the phosphorylation state of Syk protein in cell extracts.

Bone marrow-derived macrophages (BMDM) or primary human dendritic cells are starved for 4 hours in 1% serum RPMI, removed from tissue culture dishes with PBS-EDTA, washed with PBS, and counted.

Next, Lentiviral mediated transduction of the cells with vectors encoding SMART2, SMART3, SMART4, SMART5, or other chimeric receptors is performed. Monocytes are isolated from buffy coats of healthy donors following Lymphoprep gradient centrifugation and positive or negative magnetic antibody separation (Miltenyi Biotec, Leiden, Netherlands). Purity is assessed by flow cytometry of anti-CD14-PE stained cells. Isolated cells are cultured. Cells are matured to the appropriate phenotype. To ensure standardized transduction, lentiviral supernatants are titrated. Cells are plated in a 96-well plate at 50,000 cells/well and infected (50 ng p24) by spinoculation (90 min, 950 g, 32° C.), with continuous spinning in a centrifuge, in presence of 1 μM ritonavir (NIH AIDS Reagent Program, Germantown, Md.) in a final volume of 200 μL. On day 1 post-infection, medium is refreshed. Infection is measured on day 3 by flow cytometry, gating on live cells as determined by propidium iodide staining (Miltenyi Biotec).

Viral reverse transcriptase (RT) activity, quantitative real-time qPCR for viral DNA of long terminal repeat sequences, or ELISA of p24 viral protein are performed using standard techniques. Supernatant of lentiviral vector encoding a scrambled sequence or an eGFP marker gene showed an MOI of 10 when measured on 293T cells and provided over 95% transduction efficiency. This lentiviral supernatant expressed RT activity of 5,550 mU/ml (equivalent of 1 μg of p24/ml as assessed by ELISA) in previous studies. Aliquots of this supernatant are included in all subsequent reverse transcriptase activity assays to serve as a standard reference for viral production.

On day 1 post isolation, medium is replaced with fresh medium containing 50% lentiviral supernatant. RT activity of 2,750-5,550 mU/ml is used. Cells are subsequently spinoculated (90 min, 950 g, 32° C.) in the presence of polybrene (4 μg/mL; Sigma-Aldrich, Diegem, Belgium). Medium is refreshed 24 h post-transduction and cells are cultured using standard methods.

To generate virus, transfection of vectors into 293T cells is performed using standard approaches. Viral production is achieved using standard second or third-generation lentiviral transduction vector packaging production kits, such as Virapower (Life Technologies/Fisher Scientific) using the manufacturer's instructions. The titer of the viral supernatants is measured by quantification of reverse transcriptase activity via real time-PCR and expressed as equivalent p24.

Although lentiviral vectors can be inhibited in human myeloid cells, Witkowski et al. (Witkowski, Vermeire et al., PLoS One, e0133651, 2015) optimized the transduction of myeloid cells by investigating the effect of a range of parameters, including additives such as polybrene, spinoculation, and experimental timeline. This optimized protocol is subsequently used in the experiments described herein. Transduction is performed by spinoculation as described above in the presence of polybrene, which can facilitate virus-cell binding and entry. To measure transduction efficiency, a pLKO.1-derived lentiviral vector encoding an eGFP marker gene is used. Transduction efficiency, as well as the macrophage phenotype, is evaluated five days post-transduction.

The cells are then treated on ice with Abeta aggregates, Abeta multimers, or placed into wells that have been coated with plate-bound Abeta. After washing with cold PBS, cells are lysed with lysis buffer (1% v/v NP-40%, 50 Mm Tris-HCl (pH 8.0), 150 mM NaCl, 1 mM EDTA, 1.5 mM MgCl2, 10% glycerol, plus protease and phosphatase inhibitors) followed by centrifugation at 16,000 g for 10 min at 4° C. to remove insoluble materials. Lysates are then immunoprecipitated with anti-Syk Ab (N-19 for BMDM or 4D10 for human DCs, Santa Cruz Biotechnology). Precipitated proteins are fractionated by SDS-PAGE, transferred to PVDF membranes, and probed with anti-phosphotyrosine Ab (4G10, Millipore). To confirm that all substrates are adequately immunoprecipitated, immunoblots are reprobed with anti-Syk Ab (Abcam, for BMDM or Novus Biological, for human DCs). Visualization is performed with the enhanced chemiluminescence (ECL) system (GE healthcare) (Peng et al., (2010) Sci Signal., 3(122): ra38).

Cells transduced with the SMART2-SMART6 vectors, or other SMART vectors that harbor ITAM domains such as CD3Zeta or DAP12, may induce SYK phosphorylation selectively in the presence but not the absence of antigen (e.g. multimerized, aggregated, or plate-bound Abeta for SMART2-SMART6).

### Example 15: SMART Receptor Ligand-Mediated Induction of DAP12 Phosphorylation in Mouse Macrophages Expressing SMART Receptors

TREM2 signals through DAP12, leading downstream to activation of PI3K and other intracellular signals. The ability of SMART ligands to induce DAP12 activation is determined by culturing mouse macrophages expressing cognate SMART receptors and measuring the phosphorylation state of DAP12 protein in cell extracts.

Next, Lentiviral mediated transduction of the cells with vectors encoding SMART2, SMART3, SMART4, SMART5, or other chimeric receptors is performed. Monocytes are isolated from buffy coats of healthy donors following Lymphoprep gradient centrifugation and positive or negative magnetic antibody separation (Miltenyi Biotec, Leiden, Netherlands). Purity is assessed by flow cytometry of anti-CD14-PE stained cells. Isolated cells are cultured. Cells are matured to the appropriate phenotype. To ensure standardized transduction, lentiviral supernatants are titrated. Cells are plated in a 96-well plate at 50,000 cells/well and infected (50 ng p24) by spinoculation (90 min, 950 g, 32° C.), with continuous spinning in a centrifuge, in presence of 1 μM ritonavir (NIH AIDS Reagent Program, Germantown, Md.) in a final volume of 200 μL. On day 1 post-infection, medium is refreshed. Infection is measured on day 3 by flow cytometry, gating on live cells as determined by propidium iodide staining (Miltenyi Biotec).

Viral reverse transcriptase (RT) activity, quantitative real-time qPCR for viral DNA of long terminal repeat sequences, or ELISA of p24 viral protein are performed using standard techniques. Supernatant of lentiviral vector encoding a scrambled sequence or an eGFP marker gene showed an MOI of 10 when measured on 293T cells and provided over 95% transduction efficiency. This lentiviral supernatant expressed RT activity of 5,550 mU/ml (equivalent of 1 μg of p24/ml as assessed by ELISA) in previous studies. Aliquots of this supernatant are included in all subsequent reverse transcriptase activity assays to serve as a standard reference for viral production.

On day 1 post isolation, medium is replaced with fresh medium containing 50% lentiviral supernatant. RT activity of 2,750-5,550 mU/ml is used. Cells are subsequently spinoculated (90 min, 950 g, 32° C.) in the presence of polybrene (4 μg/mL; Sigma-Aldrich, Diegem, Belgium). Medium is refreshed 24 h post-transduction and cells are cultured using standard methods.

To generate virus, transfection of vectors into 293T cells is performed using standard approaches. Viral production is achieved using standard second or third-generation lentiviral transduction vector packaging production kits, such as Virapower (Life Technologies/Fisher Scientific) using the manufacturer's instructions. The titer of the viral supernatants is measured by quantification of reverse transcriptase activity via real time-PCR and expressed as equivalent p24 as described above.

Although lentiviral vectors can be inhibited in human myeloid cells, Witkowski et al. (Witkowski, Vermeire et al., PLoS One, e0133651, 2015) optimized the transduction of myeloid cells by investigating the effect of a range of parameters, including additives such as polybrene, spinoculation, and experimental timeline. This optimized protocol is subsequently used in the experiments described herein. Transduction is performed by spinoculation as described above in the presence of polybrene, which can facilitate virus-cell binding and entry. To measure transduction efficiency, a pLKO.1-derived lentiviral vector encoding an eGFP marker gene is used. Transduction efficiency, as well as the macrophage phenotype, is evaluated five days post-transduction.

Before stimulation with ligands, mouse wild-type (WT) bone marrow-derived macrophages (BMDM) and TREM2 knockout (KO) BMDM are starved for 4h in 1% serum RPMI. 15×10⁶ cells are incubated on ice for 15 min.

Cells are washed and incubated at 37° C. After stimulation with ligands, cells are lysed with lysis buffer (1% v/v NP-40%, 50 Mm Tris-HCl (pH 8.0), 150 mM NaCl, 1 mM EDTA, 1.5 mM MgCl₂, 10% glycerol, plus protease and phosphatase inhibitors), followed by centrifugation at 16,000 g for 10 min at 4° C. to remove insoluble materials. Cell lysate is immunoprecipitated with a TREM2 antibody (R&D Systems) for total DAP12, or an antibody to human IgH variable domain (for selectively isolating the SMART receptor). Precipitated proteins are fractionated by SDS-PAGE, transferred to PVDF membranes, and probed with anti-phosphotyrosine antibody (4G10, Millipore). The membrane is stripped and reprobed with anti-DAP12 antibody (Cells Signaling, D7G1X). Each cell lysate used for TREM2 immunoprecipitations contains an equal amount of proteins, as indicated by a control Ab (anti-actin, Santa Cruz). DAP12 can be phosphorylated in macrophages transduced with a pro-repair SMART such as SMART2-SMART5, in the presence of multimerized or aggregated cognate ligand such as Abeta.

### Example 16: SMART Ligand-Mediation Modulation of the Expression of Inflammatory Cell Surface Markers on Mouse or Human Macrophages Expressing Cognate Chimeric SMART Receptors

In order to validate the regulation of inflammatory marker expression by SMART vectors, mouse or human macrophages are cultured with various inflammatory mediators, and the expression of surface markers CD86 and CD206 is measured.

Macrophages are isolated from mice or humans and transduced or transfected with SMART vectors. Cells are allowed to adhere for 4 h at 37° C., and TLR agonists LPS (Salmonella abortus equi) and zymosan (Saccharomyces cerevisiae) are added at concentrations ranging from 0.01-100 ng/ml (LPS) or 0.01-10 μg/ml (zymosan). FACS analysis of CD86 and CD206 is performed on a BD FACS Canto 48 hours later. Data analysis is performed with FlowJo (TreeStar) software version 10.0.7.

Macrophages transduced with pro-repair SMART receptors and treated with inflammatory mediators IFN-γ, LPS, or Zymosan in presence of cognate ligand (e.g. multimerized, aggregated, or plate-bound Abeta for SMART2-SMART5) may express lower levels of the inflammatory receptor CD86 but not of the receptor CD206 compared to macrophages not exposed to ligand. In contrast, macrophages transduced with pro-inflammatory SMART receptors and treated with inflammatory mediators IFN-γ, LPS, or Zymosan in the presence of cognate ligand may express higher levels of the inflammatory receptor CD86 but not of the receptor CD206 compared to macrophages not exposed to ligand.

### Example 17: SMART Ligand-Mediated Increase in the Survival of Mouse or Human Myeloid Cells Expressing Cognate Chimeric SMART Receptors

To evaluate the ability of SMART receptors to induce myeloid cell survival, mouse or human macrophages are transduced with SMART receptors and cultured in the presence of inflammatory mediators, along with or in the absence of cognate SMART receptor ligands. Cell survival is subsequently measured.

Murine bone marrow precursor cells are obtained by flushing tibial and femoral marrow cells with cold PBS. After one wash with PBS, erythrocytes are lysed using ACK Lysing Buffer (Lonza), washed twice with PBS, and suspended at 0.5×10⁶ cells/ml in complete RPMI media (10% FCS, Pen/Strep, Gln, neAA) with 50 ng/ml M-CSF to produce macrophages or 10 ng/ml GM-CSF to produce dendritic cells. For M2-type macrophages, 10 ng/ml IL-4 is added to the cultured cells. For M1-type macrophages, 50 ng/ml IFN-γ is added. In some experiments LPS or zymosan is added to the cell culture at day 5 at a concentration range of 1 μg/ml-0.01 ng/ml. Recombinant cytokines are purchased from Peprotech.

Cells are transduced or transfected with a single SMART vector alone or any combination of SMART vectors. To analyze viability of bone marrow-derived macrophages, cells are prepared as above and cultured in MCSF and are exposed to multimeric SMART ligands such as Abeta oligomers or plaques, Tau fibrils, or A synuclein aggregates. Cells are either plated at 10⁵/200 μl in a 96-well plate (for viability analysis using a luciferase based-assay) or at 0.5×10⁶/1 ml in a 6-well plate (for Tripan Blue exclusion cell count) in non-tissue culture treated plates. Media containing fresh M-CSF is added at day 3. Cells are gently detached from the plates with 3 mM EDTA and counted using a Burker chamber. For FACS analysis of live cells, macrophages are cultured either in 50 ng/ml MCSF for 6 days (+MCSF) or in 50 ng/ml MCSF for 4 days before MCSF is removed for an additional 36 hrs (−MCSF). Cells are stained using CD11b antibody and DAPI. For luciferase viability assays, cell viability is measured at day 5 of culture in graded concentrations of growth factors GMCSF (dendritic cells), MCSF (M1 macrophages), or MCSF+IL-4 (M2 macrophages). Cells are directly incubated with ToxGlo reagent (Promega) and luciferase activity (luminescence) is read using an XY reader. For FACS analysis of viable macrophages cultured in the presence of inflammatory mediators IFN-γ, LPS, or zymosan, cells are collected at day 5 and stained using CD11b antibody and DAPI. After culture in MCSF with cognate ligand, a significantly higher numbers of viable (trypan blue excluded) SMART-transduced macrophages may be observed than macrophages transduced with an empty vector or a SMART vector that does not recognize the ligand. For example, for SMART2-SMART5, aggregated, multimerized, or plate bound Abeta may increase cell viability. FACS analysis may reveal that SMART-expressing macrophages, cultured with or without MCSF along with an appropriate stimulatory ligand, can display increased survival compared to cells lacking SMART vectors, as indicated by a higher percentage of live (CD11b+DAPI−) cells. For luciferase assays, SMART-expressing cells cultured in the presence of growth factors GMCSF (dendritic cells), MCSF (M1 macrophages), or MCSF+IL-4 (M2 macrophages), at any or all time points during the analysis, may survive better than cells lacking a SMART receptor or stimulating ligand, as indicated by a higher luminescence reading across the range of growth factor concentrations.

### Example 18: Impact of SMART-Expressing Myeloid Cells on Alzheimer's Disease in the 5×FAD Mouse Model

To evaluate the ability of SMART-expressing myeloid cells to prevent or reverse the development of Alzheimer's disease (AD), 5×FAD mice are used. 5×FAD mice overexpress mutant human APP (695) with the Swedish (K670N, M671L), Florida (I716V), and London (V717I) familial Alzheimer's disease (FAD) mutations, along with human PS1 harboring two FAD mutations (M146L and L286V). Both transgenes are regulated by the mouse Thy1 promoter to drive overexpression in the brain and recapitulate major features of AD. Mice are treated with myeloid cells transduced with pro-repair SMART receptors (e.g. SMART2, SMART3, SMART4, SMART5, SMART9, or SMART10 recognizing Abeta or SMART6, SMART7, or SMART8 recognizing Tau) or with control vector. After treatment, Abeta plaque load is evaluated by immunohistochemistry and ELISA of tissue extracts. The number of microglia in the brain is also evaluated. Mice are tests for reductions in cognitive deficit using the Morris Water maze (a spatial learning and memory task), the Radial Arm Water Maze (a spatial learning and memory task), the Y Maze (quantifies spontaneous alternation as a measure of spatial cognition), novelty preference in an open field, operant learning to assess learning and memory, and fear conditioning (mousebiology.org website; Wang et al., (2015) Cell. pii: S0092-8674(15)00127-0).

### Example 19: Impact of SMART-Expressing Myeloid Cells on Alzheimer's Disease in Mice Mouse Models of AD

SMART-expressing myeloid cells of the present disclosure are evaluated for their ability to improve or slow the progression of Alzheimer's disease or forms of dementia, such as vascular dementia, frontotemporal dementia, and pre-dementia conditions such as mild cognitive impairment. These studies can be performed using a transgenic animal that carries a human ApoE4 allele, animals that are transduced with a vector (e.g., retroviral vector) encoding ApoE4, or animals that are treated with ApoE4 protein. The animals display AD features pathologically and/or clinically (Kim, et al., J Neurosci 31:18007-12 [2011]).

Nine-month-old male APPswe/PS1deltaE9 mice are intraperitoneally injected 4 times every 3 d, and brain tissues are collected 24 h after the last injection. Cerebral cortical tissues are lysed by sonication (3-s pulse, 5 times, 35% amplitude) with lysis buffer (50 mM Tris-HCL, 2 mM EDTA, 1 μg/ml leupeptin, 1 μg/ml aprotinin, 0.25 mM phenylmethanesulfonyl fluoride, pH 7.4). Homogenates are centrifuged for 10 min at 14,000 RPM. Supernatants are used to measure IFN-γ and IL-1α levels using Rodent Cytokine Multi-Analyte Profile (Myriad RBM).

For ELISA assays, cortical tissues are sequentially homogenized with PBS, modified RIPA, and 5 M guanidine HCl buffer. Tissue homogenates are centrifuged at 18,000 rcf for 30 min after each extraction. The levels of Amyloid β and ApoE are measured by enzyme-linked immunosorbent assay (ELISA). For Amyloid β ELISA, HJ2 (anti-Aβ35-40) and HJ7.4 (anti-Aβ37-42) are used as capture antibodies, and HJ5.1-biotin (anti-Aβ13-28) as the detection antibody. Commercial reagent anti-ApoE monoclonal antibodies (e.g., WUE4, Calbiochem) are used for ApoE ELISA (Kim, Jiang et al., J Neurosci, 18007-18012, 2011).

For western blot assays, cortical tissues are gently lysed in PBS and modified RIPA (1% NP-40, 1% sodium deoxycholate, 25 mM Tris-HCl, 150 mM NaCl) in the presence of 1× protease inhibitor mixture (Roche). Tissue homogenates are centrifuged at 18,000 relative centrifugal force (rcf) for 30 min. Equal amounts of protein for each sample are run on 4-12% Bis-Tris XT gels (Bio-Rad) and transferred to PVDF membranes. Blots are probed with the following antibodies: ApoE (Academy Biomedical); APP (ZYMED); PS1-NTF (EMD Chemicals); β-secretase 1 (BACE1) (Cell Signaling Technology); synaptophysin (or SYP) (Sigma); glutamate receptor (GluR) 2/3/4 (Cell Signaling Technology); NMDAR2b (Cell Signaling Technology); postsynaptic density protein 95 (PSD-95) (Millipore); and tubulin (Sigma). Tubulin-normalized band intensity is quantified using NIH ImageJ software. Histology, staining, immunohistochemistry, and quantitative analysis are performed as known in the art, except that biotinylated mouse monoclonal antibody HJ3.4 (1:1000, targeted against amino acids 1-13 of the human Aβ sequence) is used to detect Amyloid β in tissue sections. For histology (Kim, Eltorai et al., J Exp Med, 2149-2156, 2012) and quantitative analysis of of Amyloid β plaques, brain hemispheres are placed in 30% sucrose before freezing and cutting on a freezing sliding microtome. Serial coronal sections at 50-μm intervals are collected from the rostral anterior commissure to caudal hippocampus. Sections are stained with biotinylated 82E1 (anti-Aβ1-16) antibody (1:500 dilution; IBL International) or X-34 dye. Stained brain sections are scanned with a NanoZoomer slide scanner (Hamamatsu Photonics) at 20° magnification setting. For quantitative analyses of 82E1-biotin and X-34 staining, scanned images are exported using NDP viewer software (Hamamatsu Photonics) and converted to 8-bit grayscale using ACDSee Pro 2 software (ACD Systems). All converted images are uniformly thresholded to highlight plaques, and then analyzed by “Analyze Particles” function in the ImageJ software (National Institutes of Health). Identified objects objects after thresholding are individually inspected to confirm the object as a plaque or not. Three brain sections per mouse, each separated by 300 μm, are used for quantification. These sections correspond approximately to sections at Bregma −1.7, −2.0, and −2.3 mm in the mouse brain atlas. The mean of three sections is used to represent a plaque load for each mouse. For analysis of Aβ plaque in in the cortex, the cortex immediately dorsal to the hippocampus is assessed. All analyses are performed in a blinded manner. For CD45 analysis, brain sections cut with a freezing sliding microtome are immunostained with anti-CD45 antibody (1:500 dilution; AbD Serotec). Stained brain sections are scanned with a NanoZoomer slide scanner (Hamamatsu Photonics) at 40° magnification setting. The percent area covered by CD45 staining is analyzed in the cortex by using NDP viewer, ACDSee Pro 2, and ImageJ software, as described in the previous section. Three brain sections per mouse, each separated by 300 μm, are used for quantification. The mean of three sections is used to estimate the area covered by immunoreactivity. All analyses are performed in a blinded fashion after stained images are thresholded to minimize false-positive signals.

For treating mice with SMART-expressing cells generated as above, or control transduced transduced cells that only harbor a fluorescent marker (e.g., GFP, Hamilton, Nat Rev Immunol, 533-544, 2008, Lebson, Nash et al., J Neurosci, 9651-9658, 2010), cells are injected into recipient mice using either intracardiac puncture for single injections, or subcutaneous vascular ports inserted into the jugular vein near the left atrium for repeated injections. In all cases 5×106 cells are injected in a volume of 100 ul. Blood is collected at multiple times from 5 min to 24 h after a single pulse of cells is injected. Cells are counted using flow cytometry with antibodies selective for the SMART receptor or a GFP marker co-transduced. The injected CD11b+ cells may clear rapidly from the circulation with a half-life of 90-240 min. Virtually all injected cells may be cleared from the circulation by 24 h after the injection. To identify whether the injected CD11b+ cells migrate to the CNS, the tissue distribution of GFP+ cells is compared after the infusion into both nontransgenic mice mice and mice with amyloid deposits in their brain (eg, 16-month-old APP_PS1 transgenic mice). Following exsanguination with saline, the numbers of SMART receptor and GFP-labeled cells in liver, spleen, lung, and brain are estimated at 1, 3, and 7 d both by flow cytometry of cell suspensions and by histological cell counts using stereology. Nontransgenic mice may have few GFP-labeled cells in the brain measured either by flow cytometry or stereology, yet APP_PS1 transgenic mouse brain may have concentrations of GFPlabeled cells at similar levels as peripheral organs (liver, spleen, lung). In most peripheral organs, the migration of labeled CD11b+ cells in the transgenic and nontransgenic mice may be comparable, with the exception of the liver, where a slight reduction of infiltrating cells may be detected in transgenic mice. In all tissues, for cells transduced with GFP only, only, the half-life of the labeled cells found within organs may be several days, with only a few cells detected 1 week after the injection. However, in the context of an appropriate SMART vector, such as as one encoding a receptor that binds Abeta in mice that harbor Abeta amyloid pathology such as 5×FAD or PS1APP, SMART cells may be maintained for longer, particularly at sites of pathology.

Treated mice may show an increase in myeloid IBA1-positive and CD11B-positive cells surrounding amyloid plaques and other neuropathology in treated mice. Amyloid pathology, as seen with congophylic stains or with Abeta IHC, may be progressively reduced in the context of time points at 1 day, 3 days, 7 days, 15 days, 28 days, 1 month, 3 months, 6 months, or longer. Other pathological changes such as neuronal loss may be slowed or reduced. Total tissue Abeta, or insoluble tissue Abeta, as quantified by ELISA assay, may be reduced in brain regions of treated mice, including hippocampus, frontal cortex, and other brain regions.

Behavioral changes that typify such mice expressing Abeta in the brain, such as spatial learning in the Morris Water Maze or contextual fear learning in the context of contextual freezing, may be reduced over this time frame. Contextual fear conditioning is tested using standard methods. In this behavioral assay, mice learn to associate a distinct context (CS: conditioned stimulus) with aversive footshock (US: unconditioned stimulus) through hippocampus-dependent mechanisms. During training, mice are placed in the conditioning chamber for 3 min and then received a footshock (0.8 mA, 2 s). After the shock delivery, mice are left in the chamber for another 30 s. Contextual fear memory is evaluated by scoring freezing behavior (the absence of all movement except for that needed for breathing) for 3 min when the mice are placed back into the same conditioning chamber 24 h after training. The automated FreezeFrame system (Coulbourn Instruments, Allentown, Pa., USA) is used to score the amount of freezing. After behavioral testing, some mice are sacrificed for immunoblotting and ELISA experiments, and others are perfused for immunohistochemistry.

Using the Novel Object Recognition and Radial Arm Test, the performance of SMART treated mice is compared to those of sex and age matched littermates, which receive no treatment or control GFP only vector transduced cells. Behavioral testing, performed on mice initiating treatment from age 4 months, 6 months, or 9 months, includes the object recognition test (ORT), which tests animals' long-term recognition memory, followed by the radial arm maze (RAM), which is a test of animals' spatial working memory. Behavioral studies are conducted at periods of 1 week, 1 month, 2 months, 3 months, or 6 months after initiating SMART cell treatment. During the acquisition session of the ORT, when the animals are allowed to explore freely the two identical objects presented in the testing arena, mice may equally interact with both objects as expected of normal mice. Following a 3-h interval, which the mice spend in their home cages, one of the two familiar objects is replaced with a novel one, and the behavior of the mice is then observed during the retention session. All tested mice groups may spend significantly more time exploring the novel object than the familiar one during the retention session, consistent with normal rodent exploratory behavior. SMART treated mice may also interact equally with two identical objects during the ORT acquisition session. However, during the retention session, SMART treated mice may spend significantly more time exploring the novel object, while control treated mice may fail to demonstrate significant preference toward the novel object. During RAM testing, SMART-treated mice may show gradual improvement in performance on consecutive testing days, making comparable number of errors while navigating through the maze. SMART-treated mice may also showed gradual improvement in performance on consecutive days of RAM testing. However, vehicle-treated mice may make significantly more errors, indicating memory impairment.

Spatial learning and reference memory is assessed with the Morris water maze. Swimming patterns are recorded with Ethovision 3.1 (Noldus Information Technology), which measures the time to reach a hidden platform during learning trials and the frequency of platform position crossings during probe tests. Deficit prevalence is calculated as behavioral test scores of treated or untreated mice that deviate from the norm: mean value (SD/2). SMART treated mice may show significantly reduced amyloid pathology, Abeta accumulation, and improved cognitive function relative to control treated animals.

### Example 20: Quantitative PCR for Persistence of SMART-Expressing Myeloid Cells in Blood or Presence in CSF

The duration of in vivo persistence of SMART-expressing myeloid cells in the circulation is determined by quantitative PCR (Q-PCR) utilizing TaqMan fluorogenic 5′ nuclease reaction. Q-PCR analysis is performed on genomic DNA extracted from PBMC obtained prior to and on days +1 and +7 following each infusion. Following a third infusion PBMC are also sampled on day +14, +21, +51 (Day +100 following stem cell rescue). Published data from Riddell et al (Srivastava and Riddell, Trends Immunol, 494-502, 2015) has demonstrated that adoptively transferred T cells are detected in the peripheral blood of study subjects one day following a cell dose of 5×10⁹ cells/m² at a frequency of 1-3 cells/100 PBMC. DNA is extracted from PBMC using the Qiagen QiAmp kit. The primers used to detect the scFvFc:ξ gene are: 5′HcFc (5′-TCTTCCTCTACACAGCAAGCTCACCGTGG-3′; SEQ ID NO:64) and 3′HuZeta (5′-GAGGGTTCTTCCTTCTCGGCTTTC-3; SEQ ID NO:65) and a 360 basepair fragment spanning the Fc-CD4-TM-zeta sequence fusion site is amplified. The TaqMan hybridization probe is FAM-5′TTCACTCTGAA GAAGATGCCTAGCC3′-TAMRA (SEQ ID NO:66). A standard curve is generated from genomic DNA isolated from a T cell clone with a single copy of integrated plasmid spiked into unmodified T cells at frequencies of 10⁻², 10⁻³, 10⁻⁵, and 10⁻⁶. A control primer/probe set specific for the human beta-globin gene is used to generate a standard curve for cell number and permits the calculation of the frequency of genetically modified clone in a PBMC sample. The beta-globin amplimers are as follows: Pco3 (5′-ACACAACTGTGTTCACTAGC-3; SEQ ID NO:67), GII (5′-GTCTCCTT AAACCTGTCTTG-3′; SEQ ID NO:68) and the Taqman probe is HEX-5′ACCTGACTCCTGAGG AGAAGTCT3′-TAMRA (SEQ ID NO:69).

### Example 21: Isolation of Monocytes from the Peripheral Blood of Mice

Six-month-old adult mice or 2 week old young mice (C57BL/6N; Charles River, Germany) are given an intraperitoneal overdose of sodium thiopental (12.5 mg; Sandoz, Austria) and perfused with 20 ml of 10 mM phosphate-buffer saline (PBS)/2.7 mM (5.5 mM) EDTA/25 mg/ml heparin, pH 7.3 through the left ventricle. The collected effluent is centrifuged at 550×g for 10 min at 4° C. The cell pellet is then resuspended in 4 ml of PBS/EDTA solution and 380 μl (40 μl/1×10⁶ target cells) of S-pluriBead suspension (pluriBead S-Bead CD11b Cell Separation KIT, pluriSelect) is added and incubated for 30 min on a pluriSelect pluriPlix at ˜10 rpm/7.5° angle at room temperature. Following the incubation, the cell suspension is poured directly onto the strainer and then washed 14× with 1 ml of wash buffer in a circular motion. Following attachment of the provided connector, tube and strainer, 1 ml of detachment buffer is carefully added to the strainer (containing the isolated CD11b target cells) and the cells are then incubated for 10 min at room temperature. Following incubation, 1 ml of wash buffer is added to the strainer and cells are separated from the beads by pipetting up and down (10×). The Luer-Lock is opened and 1 ml of wash buffer is added to allow detached CD11b+ cells to run into the provided tube. The strainer is then washed 10× with 1 ml of wash buffer. The cells are then centrifuged at 250×g for 10 min. The supernatant is carefully discarded and cells are resuspended in 100 μl of desired vehicle (e.g. FACS or infusion buffer). Approximately 10.7±0.8 million (n=8) cells are isolated from one animal.

### Example 22: Evaluation of Phagocytosis by Mouse CD11b-Positive Monocytes

The phagocytic activity of monocytes is assessed using FITC-Dextran (Sigma Aldrich, 100 ng), FluoSpheres Red (580/605) Fluorescent Microspheres (Molecular Probes, 1 μm, 3.6×107 microspheres/ml), and FITC-β-Ala-Amyloid β-Protein (1-42) (Bachem, 100 ng). Approximately 500,000 cells are resuspended in 500 μl of culture medium (MEM+1 mg/ml BSA+0.35 mg/ml NaHCO3, pH 7.2) ±1 μg/ml lipopolysaccharide (LPS) and incubated overnight at 37° C./5% CO2, then centrifuged (300×g 10 min), resuspended in 100 μl FACS flow and analyzed (BD FACS Calibur).

Cells are also characterized for their antigen expression. Following cultivation, cells are centrifuged at 300×g for 10 min, resuspended in 50 μl of FACS buffer (1% EDTA, 0.5% FCS, pH 7.1) containing primary antibodies against CD11b (1:25; BD, 557395), CD11c (1:25; Miltenyi, 130-091-842), CD14 (1:25; BD, 553739), CD45 (1:25; Miltenyi, 130-091-609), CD68 (1:5; Thermo Fisher Scientific, MA1-82739), F4/80 (1:10; Serotec/Biorad, MCA497FT), Ly6C (1:25; Miltenyi, 130-093-134), and major histocompatibility complex II (MHCII; 1:25, Miltenyi, 130-081-601) and incubated at 4° C. for 30 min. Cells are subsequently washed, centrifuged and resuspended in 100 μl of FACS Flow and analyzed. All necessary IgG (IgG2a, 2b(k) and IgG1) controls are included.

### Example 23: Intravenous Infusion of Monocytes into Alzheimer's Disease Animal Model

APPSwDI transgenic mice (C57BL/6-Tg(Thy1-APPSwDutIowa) BWevn/Mmjax; The Jackson Laboratory), expressing amyloid precursor protein (APP) harboring the Swedish K670N/M671L, Dutch E693Q, and Iowa D694N mutations, are housed and provided open access to food and water under 12 h/12 h light-dark cycles. These mice are generated and have been extensively characterized previously by Davis et al. (Davis, Xu et al., J Biol Chem, 20296-20306, 2004). All animals are genotyped according to standardized methods.

Following transfection or transduction and expression of SMART vectors or controls, male APPSwDI mice receive an intravenous (i.v.) injection via the lateral tail vein of ˜5×10⁶ CD11b-positive monocytes (obtained from 2 week old wildtype mice) in 100 μl of heparinized saline at five, six, and seven months of age. Local anesthetic (5% Emla, AstraZeneca) is applied to dampen pain prior to injection. Animals receiving saline alone serve as negative controls. At the end of the experiment, animals are anesthetized by subcutaneous sodium thiopental (12.5 mg/ml, 1 ml) injection. Blood is taken directly from the heart, collected in EDTA tubes, and centrifuged at 400×g for 10 min. Plasma is stored at −80° C. until further use. The brain is removed and a medial sagittal cut is made to divide the brain into two hemispheres. The left hemisphere is post-fixed in 4% PFA overnight and then stored in a 20% sucrose/PBS solution until further use for immunostaining Regions of the cortex in the right hemisphere are removed and immediately frozen at −80° C. for detection of inflammatory markers and Western Blots.

Cortical tissue is thawed and dissolved in 100 μl ice-cold PBS containing a protease inhibitor cocktail (P-8340, Sigma), homogenized using an ultrasonic device (Hielscher Ultrasonic Processor, Germany) and then centrifuged at 16000×g for 10 min at 4° C. The supernatant is collected and samples are stored at −80° C. until further use in ELISA and Western Blots assays. Total protein is determined by Bradford protein assay.

The detection of inflammatory proteins (monocyte chemotactic protein-1, MCP-1; macrophage inflammatory protein-2, MIP-2; tumor necrosis factor-α, TNF-α; interleukin-1β, IL-1β) is performed using the Thermo Scientific SearchLight Protein Array Technology (THP Medical Products, Vienna). Briefly, cell extracts (diluted 1:2 in diluent) or calibrated standards are added to coated wells of the provided plate and incubated for 3 h. After washing, the biotinylated antibodies are added and following 30 min incubation the wells are washed again and incubated with streptavidin-horseradish peroxidase conjugate. After the final washing step the SuperSignal Chemiluminescent Substrate is added. All incubation steps are carried out on a shaker at 20° C. The luminescent signal is detected using a compatible CCD imaging and analysis system and the absorbance is measured at 450 nm. The concentration of each sample is quantified by comparing spot intensities with the corresponding standard curves calculated from values of the standard samples using the SearchLight Array Analyst Software.

Western blot analysis is performed using standard methods. Following extraction, cells are centrifuged and 20 μl of supernatant is loaded with sample buffer. Samples are separated in 10% Bis-Tris SDS-polyacrylamide gels for 35 min at 200V and then electrotransferred to nylon-PVDF Immobilon-P^{SQ} membranes for 90 min at 30V in 20% methanol blotting buffer. The Western Breeze Chromogenic System is used for the detection of specific proteins in cortical extracts. Briefly, blots are blocked for 30 min in blocking buffer, incubated with primary antibodies against actin (1:1000; Sigma, A2066), amyloid precursor protein (APP; 1:1000; Abcam, ab32136), catalase (1:10,000; Thermo, PA1-28372), or matrix metallopeptidase 2 (MMP-2; 1:1000; Abcam, ab37150) for 90 min, washed, and then incubated in alkaline phosphatase conjugated anti-rabbit (or anti-goat) IgG for 30 min. After washing, bound antibodies are visualized by p-nitro blue tetrazolium chloride and 5-bromo-4-chloro-3-indolyl phosphate. Values for protein expression are obtained by quantifying optical density of protein bands (corrected for actin) using Image J software.

Immunohistochemistry is performed using standard methods. Following fixation, the left brain hemisphere is placed on a cork, frozen in a CO₂ stream and subsequently cut into 40-μm cryostat (Leica CM 1950) sections. Monocytes are cultivated on collagen-coated=Nunc Lab-Tek II chamber slides (Thermo Scientific) and following incubation fixed with 4% PFA for 30 min at room temperature. The brain sections or cells are then washed with PBS and incubated in PBS/0.1% Triton (T-PBS) for 30 min at 20° C. while shaking. To quench endogenous peroxidase, sections/cells are treated with PBS/1% H₂O₂/5% methanol. After incubation, the sections/cells are then blocked in T-PBS/20% horse serum (GIBCO Invitrogen)/0.2% BSA (SERVA) for 30 min at 20° C. shaking. Following blocking, brain sections/cells are incubated with Aβ [4-5 kD, Invitrogen, 1:200], Aβ [1-16, Covance, 1:1000], or ionized calcium-binding adapter molecule 1 (Iba1) in T-PBS/0.2% BSA overnight at 20° C. Cells are then incubated with DAPI (1:10,000, Sigma) and propidium iodide (PI, 1-2 μg/ml, Sigma) for 1 h in the dark (shaking) at room temperature. Slides are then washed, cover-slipped with Vectashield Mounting Medium (Vector Laboratories), and visualized under a fluorescent microscope. In the case of non-conjugated primary antibodies, sections are then washed and incubated with the corresponding biotinylated secondary antibody (1:200, Vector Laboratories) in T-PBS/0.2% BSA for 1 h at 20° C. shaking. Following secondary antibody incubation, sections are rinsed with PBS and incubated in avidin-biotin complex solution (Elite ABC kit, Vector Laboratories) for 1 h at 20° C. shaking. Finally, the sections are washed with 50 mM Tris-buffered saline (TBS) and then incubated in 0.5 mg/ml 3,3′-diaminobenzidine (DAB, Sigma)/TBS/0.003% H₂O₂ at 20° C. in the dark until a signal is detected. Once DAB staining is visible, the reaction is stopped by adding TBS to the sections. The brain sections are rinsed with TBS, mounted onto glass slides, cover-slipped with Entellan (Merck, Darmstadt, Germany), and then evaluated under the microscope by a blind observer.

Images are captured with an Olympus BX61 (ProgRes C14 camera) microscope using Openlab 5.5.0 imaging software and acquired under the same exposure settings. For quantification, two to four brain sections per animal are evaluated for cortical staining patterns under the 10× (Iba1) or 20× (Aβ) objectives. The number of Aβ-positive plaques and Iba1-positive cells are evaluated and quantified using ImageJ software (NIH). Images are normalized to the same threshold levels and converted into binary formats. Iba1-positive cells are quantified using the particle analysis tool, set at 35-1000 μm² in a 3.8 mm² area. Plaque burden is calculated using area occupied by plaques divided by total cortical region. For specific plaques sizes, plaques are counted between 25-400 μm², 400-1600 μm², and >1600 μm² within a 1.9 mm² area. Evaluations are carried out by a blinded investigator.

### Example 24: Using Monocytes Transduced with SMART Receptors to Treat the APP-PS1 Mouse Model of AD

Doubly transgenic amyloid precursor protein (APP)+presenilin-1 (PS1) mice that are a cross between the mAPP transgenic line Tg2576 and the mPS1 transgenic line 5.1 are used in these studies. This breeding produces both APP+PS1 mice and nontransgenic mice (littermates). The mice used in the green fluorescent protein (GFP) transgenic mouse model for bone marrow donors are from The Jackson Laboratory [C57BL/6-Tg(UBC-GFP)30Scha/J (stock #004353)]. These transgenic mice express GFP under the direction of the human ubiquitin C promoter. Sixteen-month-old transgenic and nontransgenic mice are used for the single injection time course study and 9-month-old APP+PS1 mice are used for the 2 month multiple injection study as below. All mice are bred and maintained in an animal facility according to institutional guidelines.

### Adoptive Transfer of Monocytes to Animal Models of Disease

CD11b+ cells are collected using standard technology to one versed in the art. Transgenic GFP mice are overdosed with pentobarbital. GFP donor mice are killed and their femurs and tibias removed aseptically. Femur and tibia marrow cavities are flushed with RPMI media containing fetal bovine serum (FBS) and HEPES, pH 7.4, using a 25 gauge needle. Single-cell suspensions are prepared by repeat pipetting and the cell preparations passed through a 70 μm nylon mesh to remove particulate matter. Three milliliters per mouse of 1×RBC lysis buffer is added to the cells and incubated at room temperature for 5 min before adding cold PBS. Cells are centrifuged, washed twice in RPMI, and counted using a hemocytometer. CD11b+ bone marrow cells are collected using Miltenyi Biotec's LS columns and MidiMacs magnet following the manufacturer's instructions. Briefly, 100-150 million bone marrow cells from transgenic mice ubiquitously expressing GFP are suspended in 2.7 ml of PBS+0.5% BSA and incubated for 15 min together with CD11b antibody conjugated to magnetic microbeads at 4° C. (Miltenyi Biotec, catalog #130-049-601). These beads can isolate cells while binding only a fraction of the antigenic sites. The cell suspension is applied to the supplied column in a magnetic field and the CD11b+ fraction is separated from the unlabeled cells by washing three times with 3 ml of buffer. The column is separated from the magnet and CD11b+ cells are collected. The purity of immunomagnetically separated and GFP-transfected cells are analyzed using a FACScan (Becton Dickinson) equipped with a 488 nm argon laser. The bone marrow cells from non-GFP C57BL/6 mice are harvested as above. To 10̂7 total cells containing 10 μl of CD11b microbeads (incubated for 15 min at 4° C.), 10 μl of anti-CD11b-FITC (a fluorochrome-conjugated antibody, Miltenyi Biotec, #130-081-201) is added and incubated for 5 min at 4° C. Cells are washed by adding 2 ml of buffer (PBS+0.5% BSA) and centrifuged at 300-400×g for 10 min. Cells are resuspended in 500 μl of buffer and transported to Flow Cytometry core for analysis.

Cells are transduced with SMART receptor-encoding vectors by the methods detailed above. Subsequently, cells are either directly transplanted, or first maintained and expanded in the presence of soluble or plate bound ligand (eg aggregated Abeta, aSynuclein, or Tau) as detailed above for 24 hrs, 48 hrs, or up to 6 days prior to reisolation by MACS separation and transplantation.

Based upon the flow cytometry information, 5×10̂6 freshly isolated CD11b+ cells or SMART-transduced cells are then resuspended in 100 μl of saline and injected into the left heart ventricle for the single injection time course studies.

### Tissue Collection and Histochemical Procedures

On the day the mice are killed, mice are overdosed with pentobarbital (100 mg/kg). The brain, spleen, liver, and lung are removed and bisected, the right half is collected for flow cytometry (see below), and the left half is immersed in freshly prepared 4% paraformaldehyde in 100 mM PO4 buffer, pH 7.4. The organs are postfixed in paraformaldehyde for 24 h. The brain, liver, and spleen tissue are cryoprotected in a series of sucrose solutions, frozen, sectioned in the horizontal plane at 25 μm using a sliding microtome, and stored at 4° C. in Dulbecco's PBS for immunocytochemistry and histology. The lung tissue is paraffin embedded before being sectioned using a rotary microtome.

Immunohistochemistry is performed on free-floating sections as is standard in the field. A series of eight sections spaced 600 μm apart are incubated with primary antibody overnight at 4° C., then incubated in the biotinylated secondary antibody (2 h) followed by streptavidin-peroxidase. Peroxidase reactions include 1.4 mM diaminobenzidine with 0.03% hydrogen peroxide in PBS for 5 min.

Single and multiple immunofluorescent labeling are performed as follows: after incubation with the primary antibody, the free-floating sections are incubated for 2 h with the appropriate fluorophore-coupled secondary antibodies [AlexaFluor 594 (1:1500), AlexaFluor 488 (1:1500), AlexaFluor 350 (1:1500) (Invitrogen]. Sections are rinsed in Dulbecco's PBS and coverslipped with VECTASHIELD Mounting Medium. The following primary antibodies are used for immunohistochemistry: CD11b (rat monoclonal anti-CD11b, Serotec), GFP (chicken anti-GFP, AbCam), 6E10 (mouse monoclonal, Covance), CD68 (rat monoclonal, Serotec), CD45 (rat anti-mouse, Abd Serotec), F4/80 (rat anti-mouse, Abd Serotec), Iba-1 (rabbit anti-IBA-1, WAKO), and hemagglutinin (mouse anti-HA rhodamine, Roche).

Congo red histology is performed using sections mounted on slides and air dried (Lebson, Nash et al., J Neurosci, 9651-9658, 2010). Rehydrated sections are incubated in an alkaline alcoholic saturated sodium chloride solution (2.5 mM NaOH in 80% alcohol, freshly prepared) for 20 min, then incubated in 0.2% Congo red in alkaline alcoholic saturated sodium chloride solution (freshly prepared and filtered) for 30 min. Sections are rinsed through three rapid changes of 100% ethanol, cleared through three changes of xylene, then coverslipped with DPX. SMART treated mice may show significantly reduced amyloid pathology, Abeta accumulation, and improved cognitive function relative to control treated animals.

### Example 25: Delivery of Immature Dendritic Cells In Vivo

A DC-enriched population is generated from bone marrow using methods know in the art. After RBC lysis and washing with RPMI (Gibco, Grand Island, N.Y., USA), cells are resuspended in freezing media (Gibco), and stored in liquid nitrogen. Upon rapid thawing, cells are washed with RPMI (Gibco) and seeded in 6-well plates at a concentration of 2×10⁻⁶ cells/mL in complete media: RPMI 1640 (Gibco), 10% fetal bovine serum (fetal bovine serum, Gibco), 2 mmol/L L-glutamine (Gibco), 1% nonessential amino acids (Gibco), 1 mmol/L sodium pyruvate (Gibco), 1% penicillin-streptomycin and the cytokines, interleukin-4, granulocyte-macrophage colony stimulating factor, F1t-3 ligand (all at 5 ng/mL/cytokine, RD Systems, Minneapolis, Minn., USA). On the third day in vitro (DIV 3), 1 mL/well of complete media is added. On DIV 4, media containing non-adherent cells is removed and replaced with 0.75 mL complete media containing 10 μg/mL protamine sulfate (Sigma, St Louis, Mo., USA) and a lentiviral vector (LV) encoding a SMART chomeric receptor (multiplicity of infection (MOI)=10 to 30). Eighteen hours post LV transduction, virus-containing media is replaced with a combination of 75% complete media and 25% spun-down conditioned media from DIV 4. On DIV 6 to 7, cultures are harvested in their media, spun at 1,300 r.p.m. at room temperature, and resuspended in RPMI.

Modified dendritic cells are then transfused. A catheter is inserted into the carotid artery and 2×10⁶ dendritic cells are infused (0.3 mL over 1 minute), 2.5×10⁻⁶ cells are injected over 1 minute, or vehicle (50% RPMI 1640/50% complete media without cytokines) is injected over 1 minute. After infusion, the catheter is removed, the artery sutured, and the wound closed.

### Example 26: SMART-Expressing Monocyte Delivery Directly to the CNS

Monocytes or macrophage harboring a CRISPR vector can be delivered directly to the CNS via delivery to the intracerebroventricular (ICV) space or intracerebrally to the site of a lesion or otherwise. For example, Bone Marrow (BM) cells are harvested from the femur and tibiae of naïve mice and enriched for mononuclear cells on a Ficoll density gradient using standard techniques. A BM monocyte population is isolated by MACS enrichment using biotinylated anti-CD11B antibodies and streptavidin-coupled magnetic beads (Miltenyi Biotec) according to the manufacturer's protocols. Cells are subsequently transduced transfected or otherwise modified to express SMART vectors that bind to Abeta, such as SMART2-SMART5 or SMART9 and SMART10. For intravenous injections, 3.5×10̂6 cells per mouse are used; for ICV administration, 0.5̂10̂6 cells per mouse are used. Cells are injected into Alzheimer model animals such as the APP transgenic mice described above. Mice treated with SMART vector modified cells but not control cells may be protected from AD-like pathology such as Abeta plaque accumulation and behavioral learning deficiency.

### Example 27: Analysis of the Effect of SMART Cells in Mouse Models of Alzheimer's Disease

The ability of cells transduced with SMART receptors to modulate the expression of inflammatory genes in the brain of APPPS1 mice is evaluated after intracranial (IC) administration of SMART-expressing cells. APPPS1 mice contain human transgenes for both APP bearing the Swedish mutation (K670N, M671L) and PSEN1 containing an L166P mutation, both under the control of the Thy1 promoter.

On the day of surgery mice are weighed, anesthetized with isoflurane, and placed in a stereotaxic apparatus (51733D digital dual manipulator mouse stereotaxic frame; Stoelting). A mid-sagittal incisionis made to expose the cranium and four burr holes are drilled with a dental drill mounted in the stereotaxic frame over the frontal cortex and hippocampus to the following coordinates: frontal cortex, anteroposterior, +1.7 mm, lateral ±2.0 mm; hippocampus, anteroposterior −2.7 mm; lateral, ±2.5 mm, all taken from bregma. A 26 gauge needle attached to a 10 ml Hamilton syringe (Hamilton) containing the solution to be injected is lowered 3.0 mm ventral to bregma, and a 2 μl injection is made over a 2 min period. The incision is cleaned and closed with surgical staples.

Three days post-injection, mice are perfused with saline and the right hemisphere of the brains is dissected into frontal cortex, hippocampus, and rest of brain, and flash frozen. RNA is extracted from the left hippocampus using the Trizol Plus RNA Purification System (Ambion, Invitrogen) according to the manufacturer's instructions. RNA is quantified using the BioSpec Nano spectrophotometer (Shimadzu) and cDNA is reverse transcribed using the cDNA High Capacity kit (Applied Biosystems) according to the manufacturer's instructions. Real-time PCR is performed using the 384-well microfluidic card custom TaqMan® assays containing TaqMan® gene expression probes for genes of interest IL-1b, IL-6, TNFα, IL-12, YM-1, IL-1Rα, MRC1, IL-10, CD86, FCGR1B, and TGFb (Applied Biosystems, Invitrogen). All gene expression data is normalized to 18S rRNA expression. Fold change is determined using ΔCT-method. Data are presented as mean±SEM. Statistical analysis is performed using the JMP statistical analysis program (SAS). Statistical significance is assigned where the p value was lower than 0.05. One-way ANOVA and two-way ANOVA are used, where appropriate, to detect treatment differences and differences within treatment groups along the time course.

Treatment with SMART receptor-expressing myeloid cells may significantly increase the expression of IL-1b, IL-6, TNFα, and CD86, such as by approximately 2-fold or more. The expression of FCGR1B and IL-10 may be increased similarly or more, and the expression of IL-10 may be increased approximately 4-fold. By contrast, expression of the IL-1Ra may be decreased.

The ability of SMART receptor-expressing myeloid cells to reduce the amount of amyloid beta (Abeta) peptide in different regions of the brain of APPPS1 mice is evaluated after intracranial (IC), ICV, or intra-arterial or IV administration of SMART receptor-expressing myeloid cells. For instance, five mice per group received an IC injection of cells as described above. For the quantification of Abeta peptide, three days pot injection, after injection with a lethal dose of pentobarbital, mice are perfused intracardially with 25 ml of normal saline. Brains are rapidly removed and bisected in the mid-sagittal plane. The left half is immersion fixed in freshly prepared 4% paraformaldehyde. The right half is dissected, with the frontal cortex and hippocampus being isolated, flash frozen in liquid nitrogen, and stored at −80° C. The left hemibrain is passed through a series of 10, 20, and 30% sucrose solutions as cryoprotection and 25 μm frozen horizontal sections are collected using a sliding microtome and stored floating in PBS containing sodium azide at 4° C. Sections spaced 300 μm spanning the estimated injection site are initially mounted and stained by cresyl violet to identify the injection site. For all subsequent histology and immunohistochemistry six sections spanning the injection site (spaced 100 μm apart) are selected and analyzed. Free-floating immunohistochemistry for Abeta (rabbit polyclonal antibody Aβ1-16; Invitrogen) is performed. The percent area occupied by positive stain is calculated using Nikon elements BR software.

Treatment with SMART expressing cells may decrease the area of the brain that stains positive for the Abeta peptide. For example, in the frontal cortex or hippocampus, Abeta peptide may be detected in ˜4% of the tissue in mice treated with control cells not expressing said vectors, as compared to ˜2% of the tissue in mice that are treated with SMART receptor-expressing myeloid cells.

To evaluate the ability of SMART receptor-expressing myeloid cells to delay, prevent, or reverse the development of Alzheimer's disease (AD), 5×FAD mice are used. 5×FAD mice overexpress mutant human APP (695) with the Swedish (K670N, M671L), Florida (I716V), and London (V717I) familial Alzheimer's disease (FAD) mutations, along with human PS1 harboring two FAD mutations, M146L and L286V. Both transgenes are regulated by the mouse Thy1 promoter to drive over expression on the brain and recapitulate major features of AD. Mice are treated starting from 14 weeks of age weekly with SMART receptor-expressing myeloid cells or with vector only transduced myeloid cells. Mice are tested for Abeta plaque load with immunohistochemistry and by ELISA of tissue extracts. Mice are further tested for the number of microglia in the brain, and for reduction in cognitive deficit using the Morris Water maze (a spatial learning and memory task), the Radial Arm Water Maze (a spatial learning and memory task), the Y Maze (quantifies spontaneous alternation as a measure of spatial cognition), novelty preference in in an open field, operant learning to assess learning and memory, and fear conditioning (mousebiology.org website; Wang et al., (2015) Cell. pii: S0092-8674(15)00127-0).

To evaluate the ability of SMART receptor-expressing myeloid cells to delay, prevent, or reverse the development of Alzheimer's disease (AD), Tg2576 mice are used. Tg2576 mice overexpress a mutant form of APP (isoform 695) bearing the Swedish mutation (KM670/671NL). Mice are treated weekly from 98-99 weeks of age with SMART receptor-expressing myeloid cells or with cells only expressing vector sequences. Mice are tested for Abeta plaque load with immunohistochemistry and by ELISA of tissue extracts. Mice are further tested for the number of microglia in the brain, and for reduction in cognitive deficit using the Morris Water maze (a spatial learning and memory task), the Radial Arm Water Maze (a spatial learning and memory task), the Y Maze (quantifies spontaneous alternation as a measure of spatial cognition), novelty preference in in an open field, operant learning to assess learning and memory, and fear conditioning (mousebiology.org website; Wang et al., (2015) Cell. pii: S0092-8674(15)00127-0).

### Example 28: Induction of CCR7 and Migration Toward CCL19 and CCL21 in SMART Vector-Modified Microglia, Macrophages, and Dendritic Cells in the Presence of Ligand

In the presence of ligand, SMART-modified myeloid cells may induce CCR7 and migration toward CCL19 and CCL21 in microglial cells, macrophages, and dendritic cells. Microglial, Microglial, macrophages or dendritic cells are either cultured with cognate ligand, such as aggregated or multimerized Abeta peptide for SMART2-5 and SMART9-SMART10, or control media only. Cells are collected after 72 h, immuno-labeled with CCR7 specific anti-bodies, and analyzed by flow cytometry. To determine any functional consequences of increased CCR7 expression, a chemotaxis assay is performed. Microglia, macrophages or dendritic cells are stimulated with ligand or media control and placed in a two-chamber system. The number of cells migrating toward the chemokine ligands CCL19 and CCL21 is quantified (JEM (2005), 201, 647-657). For the chemotaxis assay, microglial, macrophages or dendritic cells are exposed to the ligand with or without treatment with 1 μg/ml LPS. SMART expressing microglia, macrophages or dendritic cells are transferred into the upper chamber of a transwell system (3 μm pore filter; Millipore) containing 450 μl medium with 100 ng/ml CCL19 or CCL21 (both from PeproTech) in the lower chamber. After a 1 h incubation period, the number of microglia, macrophages, or dendritic cells that have migrated to the lower chamber is counted in three independent areas by microscopy (JEM (2005), 201, 647-657).

### Example 29: Ability of Connate Ligands to Increase the Survival of SMART Chimeric Receptor-Expressing Macrophages and Dendritic Cells

To evaluate the role of SMART chimeric receptors in cell survival, SMART vector-expressing macrophages and dendritic cells are cultured in the presence of cognate ligand and cell viability is determined.

Murine bone marrow precursors are obtained by flushing tibial and femoral marrow cells with cold PBS. After one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amounts of 50 ng/ml M-CSF to produce macrophages, or 10 ng/ml GM-CSF to produce dendritic cells. For M2-type macrophages, 10 ng/ml IL-4 is added to the cultured cells. For M1-type macrophages, 50 ng/ml IFN-γ is added. In some experiments LPS or zymosan is added to the cell culture at day 5 at a concentration range of 1 μg/ml-0.01 ng/ml. Recombinant cytokines are purchased from Peprotech.

To analyze viability of bone marrow-derived macrophages, cells are prepared as above and cultured in MCSF. Cells are transduced, transfected, or otherwise modified to express SMART chimeric receptors using the techniques described above. Cells harboring SMART vectors or control vector only are either plated at 10⁵/200 μl in a 96-well plate (for viability analysis using a luciferase based-assay) or at 0.5×10⁶/1 ml in a 6-well plate (for Tripan Blue exclusion cell count) in non-tissue culture treated plates. Media containing fresh M-CSF is added at day 3, with or without cognate ligand for the SMART receptors. At indicated time points cells are gently detached from the plates with 3 mM EDTA and counted using a Burker chamber. For FACS analysis of live cells, macrophages are cultured either in 50 ng/ml MCSF for 6 days (+MCSF) or in 50 ng/ml MCSF for 4 days before MCSF is removed for an additional 36 hrs (−MCSF). Cells are stained using CD11b antibody and DAPI. For luciferase viability assays, cell viability is measured at day 5 of culture in graded concentrations of growth factors GMCSF (dendritic cells), MCSF (M1 macrophages), or MCSF+IL-4 (M2 macrophages). Cells are directly incubated with ToxGlo reagent (Promega) and luciferase activity (luminescence) is determined. For FACS analysis of viable macrophages cultured in the presence of inflammatory mediators IFN-γ, LPS, or zymosan, cells are collected at day 5 and stained using CD11b antibody and DAPI. All experiments are conducted in the presence or absence of cognate ligand for the SMART receptors, such as Abeta aggregates or soluble multimers for SMART2-SMART5 and SMART9-SMART10.

1. A polynucleotide encoding a chimeric receptor, wherein the chimeric receptor comprises:
(1) an extracellular ligand-binding domain, wherein the ligand is an agent associated with a neurological disease, disorder, or injury;
(2) a transmembrane domain; and
(3) a signaling domain,
wherein binding of the ligand to the chimeric receptor expressed in an immune cell activates the signaling domain, and the activated signaling domain induces and/or enhances
(i) cell survival of the immune cell, (ii) proliferation of the immune cell, (iii) migration of the immune cell, (iv) functionality of the immune cell, or any combination thereof.

2. The polynucleotide of claim 1, wherein the polynucleotide comprises a nucleic acid sequence selected from the group consisting of SEQ ID NOs: 38-53.

3. The polynucleotide of claim 1, wherein the chimeric receptor comprises an amino acid sequence selected form the group consisting of SEQ ID NOs: 22-37.

4. The polynucleotide of claim 1, wherein the ligand-binding domain is selected from the group consisting of a single-domain antibody, a nanobody, a heavy-chain antibody, a V_{NAR} fragment, a single-chain Fv domain (scFv), a V_{L} domain linked to a V_{H} domain by a flexible linker, an antibody Fab, and an extracellular domain of a receptor.

5. The polynucleotide of claim 1, wherein the agent associated with a neurological disease, disorder, or injury is selected from the group consisting of amyloid beta, Tau protein, and alpha-synuclein.

6. The polynucleotide of claim 1, wherein the agent associated with a neurological disease, disorder, or injury is selected from the group consisting of antisense GGCCCC (G2C4) repeat-expansion RNA, amyloid beta, oligomeric amyloid beta, amyloid beta plaques, amyloid precursor protein or fragments thereof, Tau protein, phosphorylated or truncated Tau protein, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides.

7. The polynucleotide of claim 1, wherein the ligand-binding domain is selected from the group consisting of an anti-amyloid beta single-chain variable fragment (scFv) domain, an anti-tau-NFT single-chain variable fragment (scFv) domain, and an anti-alpha-synuclein single-chain variable fragment (scFv) domain.

8. The polynucleotide of claim 1, wherein the neurological disease, disorder, or injury is selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, multiple sclerosis, and CNS herpes.

9. The polynucleotide of claim 1, wherein the transmembrane domain is a transmembrane domain from a protein selected from the group consisting of a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-kappaB, a TLR (toll-like receptor), TLR5, Myd88, lymphocyte receptor chain, IL-2 receptor, IgE, IgG, CD16α, FcγRIII, FcγRII, CD28, 4-1BB, CD4, and CD8.

10. The polynucleotide of claim 1, wherein the transmembrane domain is a transmembrane domain selected from the group consisting of a CD8 transmembrane domain, a DAP12 transmembrane domain, a CASF-1R transmembrane domain, and a TLR5 transmembrane domain.

11. The polynucleotide of claim 1, wherein the signaling domain is a signaling domain are from one or more proteins selected from the group consisting of a receptor tyrosine kinase (RTK), an M-CSF receptor, CSF-1R, Kit, TIE3, an ITAM-containing protein, DAP12, DAP10, an Fc receptor, FcR-gamma, FcR-epsilon, FcR-beta, TCR-zeta, CD3-gamma, CD3-delta, CD3-epsilon, CD3-zeta, CD3-eta, CD5, CD22, CD79a, CD79b, CD66d, TNF-alpha, NF-KappaB, a TLR (toll-like receptor), TLR5, Myd88, TOR/CD3 complex, lymphocyte receptor chain, IL-2 receptor, IgE, IgG, CD16α, FcγRIII, FcγRII, CD28, 4-1BB, and any combination thereof.

12. The polynucleotide of claim 1, wherein the signaling domain is a signaling domain selected from the group consisting of a 4-1BB intracellular domain, a CD3-zeta ITAM domain, a CD3-zeta intracellular domain, a CSF-1R receptor tyrosine kinase (RTK) intracellular domain, a DAP12 intracellular domain, a TCR-zeta intracellular domain, a TLR5 intracellular domain, a CD28 intracellular domain, a DAP10 intracellular domain, an FcR-gamma intracellular domain, and any combination thereof.

13. The polynucleotide of claim 1, wherein the immune cell is an innate immune cell.

14. The polynucleotide of claim 13, wherein the innate immune cell is a myeloid cell.

15. The polynucleotide of claim 13, wherein the innate immune cell is an innate immune cell selected from the group consisting of macrophages, M1 macrophages, activated M1 macrophages, M2 macrophages, neutrophils, NK cells, dendritic cells, monocytes, osteoclasts, Langerhans cells, Kupffer cells, microglia, M1 microglia, activated M1 microglia, M2 microglia, astrocytes, A1 astrocytes, A2 astrocytes, myeloid derived suppressor cells, and any combination thereof.

16. The polynucleotide of claim 1, wherein the immune cell is an adaptive immune cell.

17. The polynucleotide of claim 16, wherein the adaptive immune cell is an adaptive immune cell selected from the group consisting of T cells, T helper cells, cytotoxic T cells, memory T cells, regulatory T cells, natural killer T cells, mucosal associate invariant T cells, gamma delta T cells, B cells, memory B cells, follicular B cells, marginal zone B cells, B-1 cells, B-2 cells, regulatory B cells, and any combination thereof.

18. The polynucleotide of claim 1, wherein the chimeric receptor further comprises a flexible linker located between the transmembrane domain and the signaling domain.

19. The polynucleotide of claim 18, wherein the flexible linker is a flexible linker selected from the group consisting of a CD8 hinge domain, a TLR5 hinge domain, and a CSF-1R linker domain.

20. The polynucleotide of claim 1, wherein the chimeric receptor further comprises a signal peptide at the N-terminus of the chimeric receptor.

21. The polynucleotide of claim 20, wherein the signal peptide is a CD8 secretory signal peptide.

22. The polynucleotide of claim 1, wherein the chimeric receptor further comprises a heterodimerization domain.

23. The polynucleotide of claim 22, wherein the heterodimerization domain is an inducible heterodimerization domain.

24. The polynucleotide of claim 23, wherein the heterodimerization domain is a FK506 binding protein (FKBP) heterodimerization domain.

25. The polynucleotide of claim 23, wherein the heterodimerization domain is a T2089L mutant of FKBP-rapamycin binding domain (FRB*) heterodimerization domain.

26. The polynucleotide of claim 1, wherein binding of the ligand to the chimeric receptor expressed in the immune cell induces one or more activities selected from the group consisting of:
a. TREM1 phosphorylation;
b. DAP12 phosphorylation;
c. activation of one or more tyrosine kinases;
d. activation of phosphatidylinositol 3-kinase (PI3K);
e. activation of protein kinase B;
f. recruitment of phospholipase C-gamma (PLC-gamma) to a cellular plasma membrane, activation of PLC-gamma, or both;
g. recruitment of TEC-family kinase dVav to a cellular plasma membrane;
h. activation of nuclear factor-kB (NF-kB);
i. inhibition of MAPK signaling;
j. phosphorylation of linker for activation of T cells (LAT), linker for activation of B cells (LAB), or both;
k. activation of IL-2-induced tyrosine kinase (Itk);
l. modulation of one or more pro-inflammatory mediators selected from the group consisting of IFN-γ, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, MCP-1, and any combination thereof;
m. modulation of one or more anti-inflammatory mediators selected from the group consisting of IL-4, IL-10, TGF-β, IL-13, IL-35, IL-16, IFN-α, IL-1Rα, VEGF, G-CSF, soluble receptors for TNF, soluble receptors for IL-6, and any combination thereof;
n. phosphorylation of extracellular signal-regulated kinase (ERK);
o. modulated expression of C-C chemokine receptor 7 (CCR7);
P. induction of microglial cell chemotaxis toward CCL19 and CCL21 expressing cells;
q. normalization of disrupted ITAM-dependent gene expression;
r. recruitment of Syk, ZAP70, or both to an ITAM complex;
s. increased activity of one or more ITAM-dependent genes or CSF-1R-dependent genes;
t. increased maturation of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof;
u. increased ability of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof to prime or modulate the function of T cells;
v. enhanced ability, normalized ability, or both of bone marrow-derived dendritic cells to prime or modulate function of antigen-specific T cells;
w. induction of osteoclast production, increased rate of osteoclastogenesis, or both;
x. increased survival of dendritic cells, macrophages, M1 macrophages, activated M1 macrophages, M2 macrophages, monocytes, osteoclasts, Langerhans cells, Kupffer cells, microglia, M1 microglia, activated M1 microglia, M2 microglia, Astrocytes, A1 astrocytes, A2 astrocytes, or any combination thereof;
y. increased function of dendritic cells, macrophages, M1 macrophages, activated M1 macrophages, M2 macrophages, microglia, M1 microglia, activated M1 microglia, M2 microglia, astrocytes, A1 astrocytes, A2 astrocytes, or any combination thereof;
z. increasing phagocytosis by dendritic cells, macrophages, M1 macrophages, activated M1 macrophages, M2 macrophages, monocytes, microglia, M1 microglia, activated M1 microglia, M2 microglia, astrocytes, A1 astrocytes, A2 astrocytes, or any combination thereof;
aa. induction of one or more types of clearance selected from the group consisting of apoptotic neuron clearance, nerve tissue debris clearance, non-nerve tissue debris clearance, bacteria clearance, other foreign body clearance, disease-causing protein clearance, disease-causing peptide clearance, disease-causing nucleic acid clearance, and any combination thereof; optionally wherein the disease-causing protein is selected from the group consisting of amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from the group consisting of DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA;
bb. induction of phagocytosis of one or more of apoptotic neurons, nerve tissue debris, non-nerve tissue debris, dysfunctional synapses, bacteria, other foreign bodies, disease-causing proteins, disease-causing peptides, disease-causing nucleic acids, or any combination thereof; optionally wherein the disease-causing protein is selected from the group consisting of amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from the group consisting of DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA;
cc. increased expression of one or more stimulatory molecules selected from the group consisting of CD83, CD86 MHC class II, CD40, and any combination thereof;
dd. modulated expression of one or more proteins selected from the group consisting of C1qa, C1qB, C1qC, C1s, C1R, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, VEGF, PDL-1, PDL-2, ICOS, B7-H3, B7-H4, OX40L, FOXP3, IDO, CD39, CD73, CD80, CD86, CD83, CD11b, CD14, CD33, Siglec-5, Siglec-7, Siglec-9, IFN-gamma, IFN-alpha,IFN-beta, IL-18, IL-12, IL-10, IL-6, IL-2, IL-1 (beta and alpha), TNF-alpha, TGF-beta, IRF1, IRF3, STAT1, STAT3, HIF1-alpha, GMZA, GMZB, GZMH, PRF1, GNLY, CXCL9, CXCL10, CCL5, CX3CL1, CCL2, MADCAM1, ICAM1, VCAM1, VEGF, GMCSF, MCSF, S1c7a2, Cxc19, Serpinb2, Ptgs2, Cxc13, Cd38, Arg1, Mg12, Retn1a, Ear11, Tmem26, Mrc1, Socs2, Ch25h, Chi313, S1c17a2, Flt1, TIM3, LAG3, CD137, GAL9, OX40, GITR, Osteopontin, MID1, AXL, ITGAX, LPL, SPP1, ATP6VoD2, SIGLECH, CD33, TMEM119, EMR1, CDH23, GLO1, RASGRF2, and any combination thereof;
ee. increased memory; and
ff. reduced cognitive deficit.

27. The polynucleotide of claim 1, wherein the polynucleotide is a DNA polynucleotide.

28. The polynucleotide of claim 1, wherein the polynucleotide is an RNA polynucleotide.

29. A vector comprising the polynucleotide of claim 1.

30. The vector of claim 29, wherein the vector is a lentiviral vector, a retroviral vector, a sleeping beauty vector, an AAV vector, or a non-viral plasmid vector.

31. An isolated chimeric receptor encoded by the polynucleotide of claim 1.

32. The chimeric receptor of claim 31, wherein the ligand-binding domain binds a ligand associated with Alzheimer's disease pathology.

33. The chimeric receptor of claim 32, wherein the ligand is Amyloid beta or tau.

34. The chimeric receptor of claim 31, wherein the ligand-binding domain binds a ligand associated with Parkinson's disease pathology.

35. The chimeric receptor of claim 34, wherein the ligand is alpha-synuclein.

36. The chimeric receptor of claim 31, wherein the ligand-binding domain binds a ligand associated with amyotrophic lateral sclerosis pathology.

37. The chimeric receptor of claim 36, wherein the ligand is a dipeptide repeat derived by RAN translation at the C9ORF72 gene.

38. An isolated host cell comprising the polynucleotide of claim 1.

39. An isolated host cell comprising the vector of claim 29.

40. An isolated host cell comprising the chimeric receptor of claim 31.

41. The isolated host cell of claim 38, wherein the isolated host cell is an immune cell.

42. The isolated host cell of claim 41, wherein the immune cell is an innate immune cell.

43. The isolated host cell of claim 42, wherein the innate immune cell is a myeloid cell.

44. The isolated host cell of claim 42, wherein the innate immune cell is selected from the group consisting of a macrophage, an M1 macrophage, an activated M1 macrophage, an M2 macrophage, a neutrophil, a NK cell, a dendritic cell, a monocyte, an osteoclast, a Langerhans cell, a Kupffer cell, a microglial cell, an M1 microglial cell, an activated M1 microglial cell, an M2 microglial cell, an astrocyte, an A1 astrocyte, and an A2 astrocyte.

45. The isolated host cell of claim 42, wherein the immune cell is an adaptive immune cell.

46. The isolated host cell of claim 45, wherein the adaptive immune cell is selected from the group consisting of a T cell, a T helper cell, a cytotoxic T cell, a memory T cell, a regulatory T cell, a natural killer T cell, a mucosal associate invariant T cell, a gamma delta T cell, a B cell, a memory B cell, a follicular B cell, a marginal zone B cell, a B-1 cell, a B-2 cell, and a regulatory B cell.

47. The isolated host cell of claim 38, wherein the host cell lacks one or more genes encoding one or more immune molecules that allow for recognition by the adaptive immune system.

48. The isolated host cell of claim 47, wherein the one or more immune molecules are MHC class I molecules, MHC class I co-receptors, MHC class II molecules, MHC class II co-receptors, or any combination thereof.

49. The isolated host cell of claim 47, wherein the one or more genes were deleted using a nuclease selected from the group consisting of a Cas9 nuclease, a TALEN, and a ZFN.

50. An isolated myeloid cell expressing the chimeric receptor of claim 31, wherein the cell phenotype is modified in vitro or in vivo by addition of pro-inflammatory or anti-inflammatory agents or cytokines selected from the group consisting of GM-CSF, MCSF, IL-1, IL4, IL10, IL12, TNFα, TGF-beta, and LPS.

51-54. (canceled)

55. A method of producing an immune cell expressing a chimeric receptor, comprising:
(a) isolating an immune cell;
(b) introducing the vector of claim 29 into the cell; and
(c) culturing the cell so that the chimeric receptor is expressed.

56. The method of claim 55, wherein the immune cell is an innate immune cell.

57. The method of claim 56, wherein the innate immune cell is a myeloid cell.

58. The method of claim 56, wherein the innate immune cell is selected from the group consisting of a macrophage, an M1 macrophage, an activated M1 macrophage, an M2 macrophage, a neutrophil, a NK cell, a dendritic cell, a monocyte, an osteoclast, a Langerhans cell, a Kupffer cell, a microglial cell, an M1 microglial cell, an activated M1 microglial cell, an M2 microglial cell, an astrocyte, an A1 astrocyte, and an A2 astrocyte.

59. The method of claim 55, wherein the immune cell is an adaptive immune cell.

60. The method of claim 59, wherein the adaptive immune cell is selected from the group consisting of a T cell, a T helper cell, a cytotoxic T cell, a memory T cell, a regulatory T cell, a natural killer T cell, a mucosal associate invariant T cell, a gamma delta T cell, a B cell, a memory B cell, a follicular B cell, a marginal zone B cell, a B-1 cell, a B-2 cell, and a regulatory B cell.

61. An isolated immune cell comprising a chimeric receptor produced by the method of claim 55.

62. The isolated cell of claim 38, wherein the cell further expresses one or more signaling factors that promote an M2 phenotype by inhibiting a TNF-alpha/NF-KappaB pathway a TLR/MyD88 pathway, or both.

63. The isolated cell of claim 62, wherein the one or more signaling factors that promote an M2 phenotype by inhibiting a TNF-alpha/NF-KappaB pathway are one or more signaling factors selected from the group consisting of a dominant negative IKK-alpha, a dominant negative IKK-alpha IKK-beta, a dominant negative IKK-alpha IKBa (IKBa-DN), a MEKK isoform, and any combination thereof.

64. The isolated cell of claim 62, wherein the one or more signaling factors that promote an M2 phenotype by inhibiting a TLR/MyD88 pathway are one or more dominant negative forms of MyD88.

65. A pharmaceutical composition comprising the polynucleotide of claim 1, and a pharmaceutically acceptable carrier.

66. A pharmaceutical composition comprising the vector of claim 29, and a pharmaceutically acceptable carrier.

67. A pharmaceutical composition comprising the chimeric receptor of claim 31, and a pharmaceutically acceptable carrier.

68. A pharmaceutical composition comprising the isolated cell of claim 38, and a pharmaceutically acceptable carrier.

69. A method of preventing, reducing risk, or treating a neurological disease, disorder, or injury comprising administering to an individual in need thereof a therapeutically effective amount of the isolated cell of claim 38.

70. A method of preventing, reducing risk, or treating a neurological disease, disorder, or injury in an individual in need thereof, comprising:
(a) obtaining a plurality of isolated immune cells;
(b) introducing the vector of claim 29 into the plurality of isolated immune cells; and
(c) administering to the individual a therapeutically effective amount of the plurality of isolated immune cells containing the vector.

71. The method of claim 70, wherein the plurality of isolated immune cells is administered peripherally into the individual without irradiation.

72. The method of claim 70, wherein the vector contained in the plurality of isolated immune cells is expressed after administration of the plurality of immune cells to the individual.

73. The method of claim 70, wherein the administered plurality of isolated immune cells infiltrates the central nervous system of the individual.

74. A method for therapeutic delivery of cells to a central nervous system for preventing, reducing risk, or treating a neurological disease, disorder, or injury in an individual in need thereof, comprising:
(a) obtaining a blood sample, a bone marrow sample, or a brain tissue sample from a suitable donor;
(b) immunolabeling a plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells in the blood sample, the bone marrow sample, or the brain tissue sample;
(c) isolating the immunolabeled plurality of cells from the blood sample, the bone marrow sample, or the brain tissue sample;
(d) introducing the vector of claim 29 into the isolated plurality of cells; and
(e) administering to the periphery of the individual, without irradiation of the individual or any portion of the individual, a therapeutically effective amount of the plurality of cells containing the vector, wherein the administered plurality of cells infiltrates the central nervous system of the individual.

75. The method of claim 73, wherein the expression of the vector in the administered cells increases recruitment of the cells across the blood-brain barrier and into the central nervous system of the individual.

76. The method of claim 73, wherein the administered cells further recruit myeloid cells having an M2-like protective phenotype or an A2-like protective phenotype into the central nervous system of the individual.

77. The method of claim 73, wherein the cells induce production of endogenous neuronal survival agents, trophic agents, or both locally in the central nervous system of the individual.

78. The method of claim 73, wherein binding of the ligand to the chimeric receptor induces an increase in myeloid cell activation, proliferation, survival, phagocytosis, and/or functionality.

79. The method of claim 70, wherein the neurological disease, disorder, or injury is Alzheimer's disease and the cells infiltrate Amyloid beta plaques in the central nervous system of the individual.

80. The method of claim 69, wherein the neurological disease, disorder, or injury is selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, multiple sclerosis, and CNS herpes.

81. The method of claim 70, wherein the cells are one or more innate immune cells.

82. The method of claim 81, wherein the one or more innate immune cells are myeloid cells.

83. The method of claim 82, wherein the one or more innate immune cells are selected from the group consisting of macrophages, M1 macrophages, activated M1 macrophages, M2 macrophages, neutrophils, NK cells, dendritic cells, monocytes, osteoclasts, Langerhans cells, Kupffer cells, microglia, M1 microglia, activated M1 microglia, M2 microglia, astrocytes, A1 astrocytes, A2 astrocytes, and any combination thereof.

84. The method of claim 70, wherein the cells are one or more adaptive immune cells.

85. The method of claim 84, wherein the one or more adaptive immune cells are selected from the group consisting of T cells, T helper cells, cytotoxic T cells, memory T cells, regulatory T cells, natural killer T cells, mucosal associate invariant T cells, gamma delta T cells, B cells, memory B cells, follicular B cells, marginal zone B cells, B-1 cells, B-2 cells, regulatory B cells, and any combination thereof.

86. The method of claim 69, wherein the administering induces one or more activities selected from the group consisting of:
a. TREM1 phosphorylation;
b. DAP12 phosphorylation;
c. activation of one or more tyrosine kinases;
d. activation of phosphatidylinositol 3-kinase (PI3K);
e. activation of protein kinase B;
f. recruitment of phospholipase C-gamma (PLC-gamma) to a cellular plasma membrane, activation of PLC-gamma, or both;
g. recruitment of TEC-family kinase dVav to a cellular plasma membrane;
h. activation of nuclear factor-kB (NF-kB);
i. inhibition of MAPK signaling;
j. phosphorylation of linker for activation of T cells (LAT), linker for activation of B cells (LAB), or both;
k. activation of IL-2-induced tyrosine kinase (Itk);
l. modulation of one or more pro-inflammatory mediators selected from the group consisting of IFN-γ, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, and MCP-1;
m. modulation of one or more anti-inflammatory mediators selected from the group consisting of IL-4, IL-10, TGF-β, IL-13, IL-35, IL-16, IFN-α, IL-1Rα, VEGF, G-CSF, soluble receptors for TNF, and soluble receptors for IL-6;
n. phosphorylation of extracellular signal-regulated kinase (ERK);
o. modulated expression of C-C chemokine receptor 7 (CCR7);
p. induction of microglial cell chemotaxis toward CCL19 and CCL21 expressing cells;
q. normalization of disrupted ITAM-dependent gene expression;
r. recruitment of Syk, ZAP70, or both to an ITAM complex;
s. increased activity of one or more ITAM-dependent genes or CSF-1R-dependent genes;
t. increased maturation of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof;
u. increased ability of dendritic cells, monocytes, microglia, M1 microglia, activated M1 microglia, and M2 microglia, macrophages, M1 macrophages, activated M1 macrophages, M2 macrophages, astrocytes, A1 astrocytes, A2 astrocytes, or any combination thereof to prime or modulate the function of T cells;
v. enhanced ability, normalized ability, or both of bone marrow-derived dendritic cells to prime or modulate function of antigen-specific T cells;
w. induction of osteoclast production, increased rate of osteoclastogenesis, or both;
x. increased survival of dendritic cells, macrophages, M1 macrophages, activated M1 macrophages, M2 macrophages, monocytes, osteoclasts, Langerhans cells, Kupffer cells, microglia, M1 microglia, activated M1 microglia, M2 microglia, Astrocytes, A1 astrocytes, A2 astrocytes, or any combination thereof;
y. increased function of dendritic cells, macrophages, M1 macrophages, activated M1 macrophages, M2 macrophages, microglia, M1 microglia, activated M1 microglia, M2 microglia, astrocytes, A1 astrocytes, A2 astrocytes, or any combination thereof;
z. increasing phagocytosis by dendritic cells, macrophages, M1 macrophages, activated M1 macrophages, M2 macrophages, monocytes, microglia, M1 microglia, activated M1 microglia, M2 microglia, astrocytes, A1 astrocytes, A2 astrocytes, or any combination thereof;
aa. induction of one or more types of clearance selected from the group consisting of apoptotic neuron clearance, nerve tissue debris clearance, non-nerve tissue debris clearance, bacteria clearance, other foreign body clearance, disease-causing protein clearance, disease-causing peptide clearance, disease-causing nucleic acid clearance, and any combination thereof; optionally wherein the disease-causing protein is selected from the group consisting of amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from the group consisting of DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA;
bb. induction of phagocytosis of one or more of apoptotic neurons, nerve tissue debris, non-nerve tissue debris, dysfunctional synapses, bacteria, other foreign bodies, disease-causing proteins, disease-causing peptides, disease-causing nucleic acids, or any combination thereof; optionally wherein the disease-causing protein is selected from the group consisting of amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin-1, ataxin-2, ataxin-3, ataxin-7, ataxin-8, ataxin-10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products; the disease-causing peptide is selected from the group consisting of DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the disease-causing nucleic acids are antisense GGCCCC (G2C4) repeat-expansion RNA;
cc. increased expression of one or more stimulatory molecules selected from the group consisting of CD83, CD86 MHC class II, CD40, and any combination thereof;
dd. modulated expression of one or more proteins selected from the group consisting of C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOX5AP, ITGAM, SLC7A7, CD4, ITGAX, PYCARD, VEGF, PDL-1, PDL-2, ICOS, B7-H3, B7-H4, OX40L, FOXP3, IDO, CD39, CD73, CD80, CD86, CD83, CD11b, CD14, CD33, Siglec-5, Siglec-7, Siglec-9, IFN-gamma, IFN-alpha,IFN-beta, IL-18, IL-12, IL-10, IL-6, IL-2, IL-1 (beta and alpha), TNF-alpha, TGF-beta, IRF1, IRF3, STAT1, STAT3, HIF1-alpha, GMZA, GMZB, GZMH, PRF1, GNLY, CXCL9, CXCL10, CCL5, CX3CL1, CCL2, MADCAM1, ICAM1, VCAM1, VEGF, GMCSF, MCSF, S1c7a2, Cxc19, Serpinb2, Ptgs2, Cxc13, Cd38, Arg1, Mg12, Retn1a, Ear11, Tmem26, Mrc1, Socs2, Ch25h, Chi313, S1c17a2, Flt1, TIM3, LAG3, CD137, GAL9, OX40, GITR, Osteopontin, MID1, AXL, ITGAX, LPL, SPP1, ATP6VoD2, SIGLECH, CD33, TMEM119, EMR1, CDH23, GLO1, RASGRF2, and any combination thereof;
ee. increased memory; and
ff. reduced cognitive deficit.

87. A method of testing efficacy of therapeutic delivery of cells to a central nervous system for preventing, reducing risk, or treating a neurological disease, disorder, or injury in an individual in need thereof, comprising:
obtaining a blood sample or a bone marrow sample from a suitable donor;
immunolabeling a plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells in the blood sample or the bone marrow sample;
isolating the immunolabeled plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells from the blood sample or the bone marrow sample;
introducing the vector of claim 29 into the isolated plurality of Cd11b/CD18+ and/or CD123+ an/or CD14+ and/or CD33+ and/or CD43+ and/or CD11b⁺, CD45^{low} or, CD11b⁺, CD45^{high} and/or CD68+ cells;
administering to the periphery or brain of the individual, without irradiation of the individual or any portion of the individual, a therapeutically effective amount of the plurality of cells containing the vector, wherein the administered plurality of cells infiltrates the locus of the neurological disease, disorder, or injury within the central nervous system of the individual;
detecting the presence of the administered plurality of cells at the locus of the neurological disease, disorder, or injury within the central nervous system of the individual; and
determining the therapeutic effect of the administered plurality of cells at the locus of the neurological disease, disorder, or injury.


SEQUENCE LISTING (70 sequences)

SEQ ID NO: 1 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 3 - protein, 459 aa, Artificial Sequence
  Synthetic Construct
LFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 4 - protein, 25 aa, Artificial Sequence
  Synthetic Construct
LFTPVVVACMSIMALLLLLLLLLLY

SEQ ID NO: 5 - protein, 434 aa, Homo sapiens
KYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 6 - protein, 25 aa, Artificial Sequence
  Synthetic Construct
LFTPVVVACMSVMSLLVLLLLLLLY

SEQ ID NO: 7 - protein, 441 aa, Mus musculus
KYKQKPKYQVRWKIIERYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDSEGDSSYKNIHLEKKYVRRDSGFSSQGVDTYVEMRPVSTSSSDSFFKQDLDKEASRPLELWDLLHFSSQVAQGMAFLASKNCIHRDVAARNVLLTSGHVAKIGDFGLARDIMNDSNYVVKGNARLPVKWMAPESIFDCVYTVQSDVWSYGILLWEIFSLGLNPYPGILVNNKFYKLVKDGYQMAQPVFAPKNIYSIMQSCWDLEPTRRPTFQQICFLLQEQARLERRDQDYANLPSSGGSSGSDSGGGSSGGSSSEPEEESSSEHLACCEPGDIAQPLLQPNNYQFC

SEQ ID NO: 8 - protein, 31 aa, Artificial Sequence
  Synthetic Construct
DEEEVLKSLKFSLFIVCTVTLTLFLMTILTV

SEQ ID NO: 9 - protein, 198 aa, Homo sapiens
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 10 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 21 aa, Homo sapiens
MALPVTALLLPLALLLHAARP

SEQ ID NO: 12 - protein, 18 aa, Artificial Sequence
  Synthetic Construct
GSTSGSGKPGSGEGSTKG

SEQ ID NO: 13 - protein, 244 aa, Artificial Sequence
  Synthetic Construct
EVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKR

SEQ ID NO: 14 - protein, 47 aa, Homo sapiens
TTTPAPRPPTPAPTIASQPLSLRPEACRPAAGGAVHTRGLDFACDIY

SEQ ID NO: 15 - protein, 22 aa, Homo sapiens
IWAPLAGTCGVLLLSLVITLYC

SEQ ID NO: 16 - protein, 42 aa, Homo sapiens
KRGRKKLLYIFKQPFMRPVQTTQEEDGCSCRFPEEEEGGCEL

SEQ ID NO: 17 - protein, 92 aa, Homo sapiens
LRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 18 - protein, 240 aa, Artificial Sequence
  Synthetic Construct
EVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSAGGGGSGGGGSGGGGSDIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELK

SEQ ID NO: 19 - protein, 235 aa, Artificial Sequence
  Synthetic Construct
EVQLVQSGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTSSGGGGSGGGGSGGGGSQSVLTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVL

SEQ ID NO: 20 - protein, 218 aa, Artificial Sequence
  Synthetic Construct
SRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 21 - protein, 96 aa, Artificial Sequence
  Synthetic Construct
SRILWHEMWHEGLEEASRLYFGERNVKGMFEVLEPLHAMMERGPQTLKETSFNQAYGRDLMEAQEWCRKYMKSGNVKDLLQAWDLYYHVFRRISKT

SEQ ID NO: 22 - protein, 447 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 768 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRTTTPAPRPPTPAPTIASQPLSLRPEACRPAAGGAVHTRGLDFACDIYIWAPLAGTCGVLLLSLVITLYCKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 24 - protein, 881 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRTTTPAPRPPTPAPTIASQPLSLRPEACRPAAGGAVHTRGLDFACDIYIWAPLAGTCGVLLLSLVITLYCKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 404 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRTTTPAPRPPTPAPTIASQPLSLRPEACRPAAGGAVHTRGLDFACDIYLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 26 - protein, 400 aa, Artificial Sequence
  Synthetic Construct
MALPVTALLLPLALLLHAARPEVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSAGGGGSGGGGSGGGGSDIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELKTTTPAPRPPTPAPTIASQPLSLRPEACRPAAGGAVHTRGLDFACDIYLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 27 - protein, 443 aa, Artificial Sequence
  Synthetic Construct
MALPVTALLLPLALLLHAARPEVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSAGGGGSGGGGSGGGGSDIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELKTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 764 aa, Artificial Sequence
  Synthetic Construct
MALPVTALLLPLALLLHAARPEVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSAGGGGSGGGGSGGGGSDIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELKTTTPAPRPPTPAPTIASQPLSLRPEACRPAAGGAVHTRGLDFACDIYIWAPLAGTCGVLLLSLVITLYCKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 29 - protein, 724 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 30 - protein, 837 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - protein, 438 aa, Artificial Sequence
  Synthetic Construct
MALPVTALLLPLALLLHAARPEVQLVQSGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTSSGGGGSGGGGSGGGGSQSVLTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 759 aa, Artificial Sequence
  Synthetic Construct
MALPVTALLLPLALLLHAARPEVQLVQSGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTSSGGGGSGGGGSGGGGSQSVLTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLTTTPAPRPPTPAPTIASQPLSLRPEACRPAAGGAVHTRGLDFACDIYIWAPLAGTCGVLLLSLVITLYCKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 33 - protein, 395 aa, Artificial Sequence
  Synthetic Construct
MALPVTALLLPLALLLHAARPEVQLVQSGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTSSGGGGSGGGGSGGGGSQSVLTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLTTTPAPRPPTPAPTIASQPLSLRPEACRPAAGGAVHTRGLDFACDIYLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 34 - protein, 594 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 35 - protein, 256 aa, Artificial Sequence
  Synthetic Construct
MALPVTALLLPLALLLHAARPTTTPAPRPPTPAPTIASQPLSLRPEACRPAAGGAVHTRGLDFACDIYLRPVQAQAQSDCSCSTVSPGVLAGIVMGDLVLTVLIALAVYFLGRLVPRGRGAAEAATRKQRITETESPYQELQGQRSDVYSDLNTQRPYYKSRILWHEMWHEGLEEASRLYFGERNVKGMFEVLEPLHAMMERGPQTLKETSFNQAYGRDLMEAQEWCRKYMKSGNVKDLLQAWDLYYHVFRRISKT

SEQ ID NO: 36 - protein, 552 aa, Artificial Sequence
  Synthetic Construct
MALPVTALLLPLALLLHAARPEVQLHQSGPELVKPGASVKISCKTSGYTFTEYTMHWVKQSHGKSLEWIGGVNPYNDNTSYIRKLQGKVTLTVDRSSSTAYMELRSLTSEDSAVYFCARYGGLRPYYFPMDFWGQGTSVTVSSGGGGSGGGGSGGGGSQVVLSQSPAILSASPGERVTLTCRASSSVNYMHWYQQKPGSSPKAWIYATSNLASGVPDRFSASGSGTSYSLTISRVEAEDAATYYCQQWRTNPPTFGAGTKLELKRTTTPAPRPPTPAPTIASQPLSLRPEACRPAAGGAVHTRGLDFACDIYIWAPLAGTCGVLLLSLVITLYCSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 37 - protein, 689 aa, Artificial Sequence
  Synthetic Construct
MALPVTALLLPLALLLHAARPLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFCSRILWHEMWHEGLEEASRLYFGERNVKGMFEVLEPLHAMMERGPQTLKETSFNQAYGRDLMEAQEWCRKYMKSGNVKDLLQAWDLYYHVFRRISKTRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - DNA, 1458 bp, Artificial Sequence
  Synthetic Construct
atggctctgcctgtgacagctctgctgctgcctctggccctgctgctgcatgccgccagacccgatatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcaccagcgtgaccgtgacaacaacacccgcccctagacctccaacccctgccccaacaatcgccagccagcctctgtctctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctaccagcagggacagaaccagctgtacaacgagctgaacctgggcagacgggaagagtacgacgtgctggacaagcggagaggccgggatcctgaaatgggcggcaagccccagcggagaaagaaccctcaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgccccctagataa

SEQ ID NO: 39 - DNA, 2307 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggaccaccacccctgcccctagaccacctacacccgccccaacaatcgccagccagcctctgtctctgaggcccgaggcttgtagaccagctgctggcggagccgtgcacacaagaggactggacttcgcctgcgacatctacatctgggcccctctggccggcacatgtggcgtgctgctgctgagcctcgtgatcaccctgtactgcaagtacaagcagaaacccaagtaccaagtgcggtggaagatcatcgagagctacgagggcaacagctacacattcatcgaccccacccagctgccctacaatgagaagtgggagttcccccggaacaacctgcagttcggcaagacactgggcgctggcgcctttggcaaagtggtggaagccacagccttcggcctgggcaaagaggatgccgtgctgaaggtggccgtgaaaatgctgaagtccaccgcccacgccgacgagaaagaggccctgatgagcgagctgaagatcatgagccacctgggccagcacgagaacatcgtgaatctgctgggcgcctgcacccacggcggacctgtgctcgtgattaccgagtactgctgctacggcgacctgctgaacttcctgcggagaaaggccgaggccatgctgggcccttctctgtcccctggacaggatccagagggcggcgtggactacaagaacatccacctggaaaagaaatacgtgcggcgggacagcggctttagcagccagggcgtggacacctacgtggaaatgaggcccgtgtccaccagcagcaacgacagcttcagcgagcaggacctggacaaagaagatggcagacccctggaactgagggatctgctgcacttcagcagtcaggtggcccagggcatggcctttctggcctccaagaactgcatccaccgggacgtggccgccagaaatgtgctgctgaccaacggacacgtggccaagatcggcgactttggcctggcccgggacatcatgaacgacagcaactatatcgtgaagggcaacgcccggctgcccgtgaaatggatggcccccgagagcatcttcgactgcgtgtacaccgtgcagagcgacgtgtggtcctatggcatcctgctgtgggagatcttctccctgggcctgaacccttaccccggcatcctcgtgaacagcaagttctacaagctcgtgaaggacggctaccagatggcccagcctgccttcgcccccaagaatatctacagcatcatgcaggcctgttgggccctggaacccacccacagacctaccttccagcagatctgtagctttctgcaggaacaggctcaggaagatcggagagagcgggactacaccaacctgcccagctcctctagaagcggcggctctggaagcagcagctccgagctggaagaagagtccagcagcgagcacctgacctgttgcgagcagggcgatattgcccagcccctgctgcagcccaacaactaccagttctgctga

SEQ ID NO: 40 - DNA, 2646 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggaccaccacccctgcccctagaccacctacacccgccccaacaatcgccagccagcctctgtctctgaggcccgaggcttgtagaccagctgctggcggagccgtgcacacaagaggactggacttcgcctgcgacatctacatctgggcccctctggccggcacatgtggcgtgctgctgctgagcctcgtgatcaccctgtactgcaagtacaagcagaaacccaagtaccaagtgcggtggaagatcatcgagagctacgagggcaacagctacacattcatcgaccccacccagctgccctacaatgagaagtgggagttcccccggaacaacctgcagttcggcaagacactgggcgctggcgcctttggcaaagtggtggaagccacagccttcggcctgggcaaagaggatgccgtgctgaaggtggccgtgaaaatgctgaagtccaccgcccacgccgacgagaaagaggccctgatgagcgagctgaagatcatgagccacctgggccagcacgagaacatcgtgaatctgctgggcgcctgcacccacggcggacctgtgctcgtgattaccgagtactgctgctacggcgacctgctgaacttcctgcggagaaaggccgaggccatgctgggcccttctctgtcccctggacaggatccagagggcggcgtggactacaagaacatccacctggaaaagaaatacgtgcggcgggacagcggctttagcagccagggcgtggacacctacgtggaaatgaggcccgtgtccaccagcagcaacgacagcttcagcgagcaggacctggacaaagaagatggcagacccctggaactgagggatctgctgcacttcagcagtcaggtggcccagggcatggcctttctggcctccaagaactgcatccaccgggacgtggccgccagaaatgtgctgctgaccaacggacacgtggccaagatcggcgactttggcctggcccgggacatcatgaacgacagcaactatatcgtgaagggcaacgcccggctgcccgtgaaatggatggcccccgagagcatcttcgactgcgtgtacaccgtgcagagcgacgtgtggtcctatggcatcctgctgtgggagatcttctccctgggcctgaacccttaccccggcatcctcgtgaacagcaagttctacaagctcgtgaaggacggctaccagatggcccagcctgccttcgcccccaagaatatctacagcatcatgcaggcctgttgggccctggaacccacccacagacctaccttccagcagatctgtagctttctgcaggaacaggctcaggaagatcggagagagcgggactacaccaacctgcccagctcctctagaagcggcggctctggaagcagcagctccgagctggaagaagagtccagcagcgagcacctgacctgttgcgagcagggcgatattgcccagcccctgctgcagcccaacaactaccagttctgcagagtgaagttctccagaagcgccgacgcccctgcctaccagcagggacagaaccagctgtacaacgagctgaacctgggcagacgggaagagtacgacgtgctggacaagcggagaggcagggaccctgagatgggcggaaagccccagagaagaaagaacccccaggaaggcctgtataacgaactgcagaaagacaagatggccgaagcctacagcgagatcggcatgaagggcgagcggagaagaggcaagggccacgatggcctgtaccagggcctgagcaccgccaccaaggatacctacgacgccctgcacatgcaggctctgccccccagataa

SEQ ID NO: 41 - DNA, 1215 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggaccaccacccctgcccctagaccacctacacccgccccaacaatcgccagccagcctctgtctctgaggcccgaggcttgtagaccagctgctggcggagccgtgcacacaagaggactggacttcgcctgcgacatctacctgaggcctgtgcaggcccaggcccagagcgattgcagctgtagcacagtgtccccaggcgtgctggccggaatcgtgatgggagatctggtgctgaccgtgctgatcgccctggccgtgtatttcctgggcagactggtgcctagaggcaggggagctgccgaagccgccaccagaaagcagcggatcaccgagacagagagcccctaccaggaactgcagggacagcggagcgacgtgtacagcgacctgaacacccagcgcccctactacaagtga

SEQ ID NO: 42 - DNA, 1203 bp, Artificial Sequence
  Synthetic Construct
atggctctgcctgtgacagctctgctgctgcctctggccctgctgctgcatgccgctagacccgaagtgaagctggtggaatctggcggcgacctcgtgaagcctggcggctctctgaagctgagctgtgccgccagcggcttcaccttcagcagctacgccatgagctgggtgcgccagaaccccgagaagcggctggaatgggtggccagcatcagcaagggcggcaacacctactaccccaacagcgtgaagggccggttcaccatctcccgggacaacgcccggaacatcctgtacctgcagatgagcagcctgcggagcgaggacaccgccctgtactattgtgccagaggctggggcgactacggatggttcgcctattggggccaagtgaccctcgtgaccgtgtctgctggcggcggaggatctggcggaggcggaagtggcggagggggcagcgatatcaagatgacccagagccccagctctatgtacgccagcctgggcgagcgcgtgaccatcacctgtaaagccagccaggacatcaaccgctacctgaactggttccagcagaagcccggcaagagccccaagaccctgatctaccgggccaacagactgctggatggcgtgcccagcagattcagcggctctggcagcggccaggactacagcctgacaatcagcagcctggactacgaggacatgggcatctactactgcctgcagtacgacgagttccccctgacctttggcgacggcaccaagctggaactgaaaaccaccacccctgcccccagacctccaaccccagcccctacaattgccagccagccactgagcctgaggcccgaggcttgtagacctgctgcaggcggagccgtgcacaccagaggactggatttcgcctgcgacatctacctgcggcctgtgcaggctcaggcccagagcgattgcagctgctctaccgtgtctccaggcgtgctggccggaatcgtgatgggcgatctggtgctgaccgtgctgattgccctggccgtgtacttcctgggcagactggtgcctagaggcagaggcgctgccgaagccgccaccagaaagcagagaatcaccgagacagagagcccctaccaggaactgcagggacagcggagcgacgtgtacagcgacctgaacacccagcggccctactacaagtga

SEQ ID NO: 43 - DNA, 1332 bp, Artificial Sequence
  Synthetic Construct
atggctctgcctgtgacagctctgctgctgcctctggccctgctgctgcatgccgctagacccgaagtgaagctggtggaatctggcggcgacctcgtgaagcctggcggctctctgaagctgagctgtgccgccagcggcttcaccttcagcagctacgccatgagctgggtgcgccagaaccccgagaagcggctggaatgggtggccagcatcagcaagggcggcaacacctactaccccaacagcgtgaagggccggttcaccatctcccgggacaacgcccggaacatcctgtacctgcagatgagcagcctgcggagcgaggacaccgccctgtactattgtgccagaggctggggcgactacggatggttcgcctattggggccaagtgaccctcgtgaccgtgtctgctggcggcggaggatctggcggaggcggaagtggcggagggggcagcgatatcaagatgacccagagccccagctctatgtacgccagcctgggcgagcgcgtgaccatcacctgtaaagccagccaggacatcaaccgctacctgaactggttccagcagaagcccggcaagagccccaagaccctgatctaccgggccaacagactgctggatggcgtgcccagcagattcagcggctctggcagcggccaggactacagcctgacaatcagcagcctggactacgaggacatgggcatctactactgcctgcagtacgacgagttccccctgacctttggcgacggcaccaagctggaactgaaaaccaccacccctgcccccagacctccaaccccagcccctacaattgccagccagccactgagcctgaggcccgaggcttgtagacctgctgcaggcggagccgtgcacaccagaggactggatttcgcctgcgacatctacatctgggcccctctggccggcacatgcggagtgctgctgctgagcctcgtgatcaccctgtactgtagagtgaagttcagccgcagcgccgacgcccctgcatatcagcagggacagaaccagctgtacaacgagctgaacctgggcagacgggaagagtacgacgtgctggacaagcggagaggcagggaccctgagatgggcggaaagccccagcggagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgccctgcacatgcaggccctgcctcccaggtaa

SEQ ID NO: 44 - DNA, 2295 bp, Artificial Sequence
  Synthetic Construct
atggctctgcctgtgacagctctgctgctgcctctggccctgctgctgcatgccgctagacccgaagtgaagctggtggaatctggcggcgacctcgtgaagcctggcggctctctgaagctgagctgtgccgccagcggcttcaccttcagcagctacgccatgagctgggtgcgccagaaccccgagaagcggctggaatgggtggccagcatcagcaagggcggcaacacctactaccccaacagcgtgaagggccggttcaccatctcccgggacaacgcccggaacatcctgtacctgcagatgagcagcctgcggagcgaggacaccgccctgtactattgtgccagaggctggggcgactacggatggttcgcctattggggccaagtgaccctcgtgaccgtgtctgctggcggcggaggatctggcggaggcggaagtggcggagggggcagcgatatcaagatgacccagagccccagctctatgtacgccagcctgggcgagcgcgtgaccatcacctgtaaagccagccaggacatcaaccgctacctgaactggttccagcagaagcccggcaagagccccaagaccctgatctaccgggccaacagactgctggatggcgtgcccagcagattcagcggctctggcagcggccaggactacagcctgacaatcagcagcctggactacgaggacatgggcatctactactgcctgcagtacgacgagttccccctgacctttggcgacggcaccaagctggaactgaaaaccaccacccctgcccccagacctccaaccccagcccctacaattgccagccagccactgagcctgaggcccgaggcttgtagacctgctgcaggcggagccgtgcacaccagaggactggatttcgcctgcgacatctacatctgggcccctctggccggcacatgcggagtgctgctgctgagcctcgtgatcaccctgtactgcaagtacaagcagaaacccaagtaccaagtgcggtggaagatcatcgagagctacgagggcaacagctacaccttcatcgaccccacccagctgccctacaacgagaagtgggagttccccagaaacaacctgcagttcggcaagacactgggcgctggcgcctttggcaaagtggtggaagccacagccttcggcctgggcaaagaggacgccgtgctgaaagtggccgtgaagatgctgaagtccaccgcccacgccgacgagaaagaggccctgatgagcgagctgaagatcatgagccacctgggccagcacgagaacatcgtgaacctgctgggagcctgcacccacggcggacctgtgctcgtgattaccgagtactgctgctacggcgacctgctgaacttcctgcggagaaaggccgaggccatgctgggcccttctctgtcccctggacaggatccagagggcggcgtggactacaagaacatccacctggaaaagaaatacgtgcggcgggacagcggctttagcagccagggcgtggacacctacgtggaaatgaggcccgtgtccaccagcagcaacgacagcttctccgagcaggacctggacaaagaagatggcagacccctggaactgcgggatctgctgcatttcagtagccaggtggcccagggcatggcctttctggccagcaagaactgcatccaccgggacgtggccgccagaaacgtgctgctgacaaacggacacgtggccaagatcggcgactttggcctggcccgggacatcatgaacgacagcaactacattgtgaagggcaacgccagactgcccgtgaagtggatggcccccgagagcatcttcgactgcgtgtacaccgtgcagagcgacgtgtggtcctacggcatcctgctgtgggagatcttctccctgggcctgaacccctaccctggcatcctcgtgaacagcaagttctacaagctcgtgaaggacggctaccagatggcccagcctgccttcgcccccaagaatatctacagcatcatgcaggcctgttgggccctggaacccacccacagacctacctttcagcagatctgtagctttctgcaggaacaggcccaggaagatcggagagagcgggactacaccaacctgcccagctcctctagaagcggcggcagcggatctagcagctccgagctggaagaggaaagcagcagcgagcacctgacctgttgcgagcagggcgatattgctcagcctctgctgcagcccaacaactaccagttctgctga

SEQ ID NO: 45 - DNA, 2175 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggctgttcacccccgtggtggtggcctgcatgagcatcatggcactcctcttgcttttgctcctcctgctgctgtacaagtacaagcagaaacccaagtaccaagtgcggtggaagatcatcgagagctacgagggcaacagctacacattcatcgaccccacccagctgccctacaatgagaagtgggagttcccccggaacaacctgcagttcggcaagacactgggcgctggcgcctttggcaaagtggtggaagccacagccttcggcctgggcaaagaggatgccgtgctgaaggtggccgtgaaaatgctgaagtccaccgcccacgccgacgagaaagaggccctgatgagcgagctgaagatcatgagccacctgggccagcacgagaacatcgtgaatctgctgggcgcctgcacccacggcggacctgtgctcgtgatcacagagtactgctgctacggcgacctgctgaacttcctgcggagaaaggccgaggccatgctgggcccttctctgtcccctggacaggatccagagggcggcgtggactacaagaacatccacctggaaaagaaatacgtgcggcgggacagcggctttagcagccagggcgtggacacctacgtggaaatgaggcccgtgtccaccagcagcaacgacagcttcagcgagcaggacctggacaaagaagatggcagacccctggaactgagggatctgctgcacttcagcagtcaggtggcccagggcatggcctttctggcctccaagaactgcatccaccgggacgtggccgccagaaacgtgctgctgacaaacggacacgtggccaagatcggcgactttggcctggcccgggacatcatgaacgacagcaactatatcgtgaagggcaacgcccggctgcccgtgaaatggatggcccccgagagcatcttcgactgcgtgtacaccgtgcagagcgacgtgtggtcctatggcatcctgctgtgggagatcttctccctgggcctgaacccttaccccggcatcctcgtgaacagcaagttctacaagctcgtgaaggacggctaccagatggcccagcctgccttcgcccccaagaatatctacagcattatgcaggcctgttgggccctggaacccacccacagacctaccttccagcagatctgtagctttctgcaggaacaggctcaggaagatcggagagagcgggactacaccaacctgcccagctcctctagaagcggcggctctggaagcagcagctccgagctggaagaagagtccagcagcgagcacctgacctgttgcgagcagggcgatattgcccagcccctgctgcagcccaacaactaccagttctgctga

SEQ ID NO: 46 - DNA, 2514 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggctgttcacccccgtggtggtggcctgcatgagcatcatggcactcctcttgcttttgctcctcctgctgctgtacaagtacaagcagaaacccaagtaccaagtgcggtggaagatcatcgagagctacgagggcaacagctacacattcatcgaccccacccagctgccctacaatgagaagtgggagttcccccggaacaacctgcagttcggcaagacactgggcgctggcgcctttggcaaagtggtggaagccacagccttcggcctgggcaaagaggatgccgtgctgaaggtggccgtgaaaatgctgaagtccaccgcccacgccgacgagaaagaggccctgatgagcgagctgaagatcatgagccacctgggccagcacgagaacatcgtgaatctgctgggcgcctgcacccacggcggacctgtgctcgtgatcacagagtactgctgctacggcgacctgctgaacttcctgcggagaaaggccgaggccatgctgggcccttctctgtcccctggacaggatccagagggcggcgtggactacaagaacatccacctggaaaagaaatacgtgcggcgggacagcggctttagcagccagggcgtggacacctacgtggaaatgaggcccgtgtccaccagcagcaacgacagcttcagcgagcaggacctggacaaagaagatggcagacccctggaactgagggatctgctgcacttcagcagtcaggtggcccagggcatggcctttctggcctccaagaactgcatccaccgggacgtggccgccagaaacgtgctgctgacaaacggacacgtggccaagatcggcgactttggcctggcccgggacatcatgaacgacagcaactatatcgtgaagggcaacgcccggctgcccgtgaaatggatggcccccgagagcatcttcgactgcgtgtacaccgtgcagagcgacgtgtggtcctatggcatcctgctgtgggagatcttctccctgggcctgaacccttaccccggcatcctcgtgaacagcaagttctacaagctcgtgaaggacggctaccagatggcccagcctgccttcgcccccaagaatatctacagcattatgcaggcctgttgggccctggaacccacccacagacctaccttccagcagatctgtagctttctgcaggaacaggctcaggaagatcggagagagcgggactacaccaacctgcccagctcctctagaagcggcggctctggaagcagcagctccgagctggaagaagagtccagcagcgagcacctgacctgttgcgagcagggcgatattgcccagcccctgctgcagcccaacaactaccagttctgcagagtgaagttctccagaagcgccgacgcccctgcctaccagcagggacagaaccagctgtacaacgagctgaacctgggcagacgggaagagtacgacgtgctggacaagcggagaggcagggaccctgagatgggcggaaagccccagagaagaaagaacccccaggaaggcctgtataacgaactgcagaaagacaagatggccgaagcctacagcgagatcggcatgaagggcgagcggagaagaggcaagggccacgatggcctgtaccagggcctgagcaccgccaccaaggatacctacgacgccctgcacatgcaggctctgccccccagataa

SEQ ID NO: 47 - DNA, 1317 bp, Artificial Sequence
  Synthetic Construct
atggctctgcctgtgacagctctgctgctgcctctggccctgctgctgcatgccgctagacctgaagtgcagctggtgcagtctggcggcggactggtggaacctggcggaagcctgagactgagctgtgccgtgtccggcttcgacttcgagaaggcctggatgagctgggtgcgccaggctcctggacagggactgcagtgggtggccagaatcaagagcacagccgatggcggcaccaccagctatgctgcccctgtggaaggccggttcatcatcagccgggacgacagccggaacatgctgtacctgcagatgaacagcctgaaaaccgaggacaccgccgtgtactactgcacctctgcccactggggccagggcacactcgtgacatctagcggaggcggaggatcaggcggcggaggaagtggcggagggggatctcagtctgtgctgacccagcctcctagcgtgtccgtgtctccaggccagaccgccaggatcacatgttctggcgaggccctgcccatgcagttcgcccactggtatcagcagaggcccggaaaggcccccgtgatcgtggtgtacaaggacagcgaaagacccagcggcgtgcccgagagattttccggctctagcagcggcacaaccgccaccctgacaatcactggcgtgcaggccgaggacgaggccgactactactgccagagccccgacagcaccaacacatacgaggtgttcggcggaggcaccaagctgaccgtgctgacaacaacccctgcccccagacctcctaccccagcccctacaattgccagccagcctctgagcctgaggcccgaggcttgtagaccagctgctggcggagccgtgcacacaagaggactggacttcgcctgcgacatctacatctgggcccctctggccggcacatgtggcgtgctgctgctgagcctcgtgatcaccctgtactgtagagtgaagttcagcagaagcgccgacgcccctgcctaccagcagggacagaaccagctgtacaacgagctgaacctgggcagacgggaagagtacgacgtgctggacaagcggagaggcagggaccctgagatgggcggaaagccccagagaagaaagaacccccaggaaggcctgtataacgaactgcagaaagacaagatggccgaggcctacagcgagatcggcatgaagggcgagcggagaagaggcaagggccacgatggcctgtaccagggcctgagcaccgccaccaaggacacctatgacgcactgcacatgcaggctctgccccccagatga

SEQ ID NO: 48 - DNA, 2280 bp, Artificial Sequence
  Synthetic Construct
atggctctgcctgtgacagctctgctgctgcctctggccctgctgctgcatgccgctagacctgaagtgcagctggtgcagtctggcggcggactggtggaacctggcggaagcctgagactgagctgtgccgtgtccggcttcgacttcgagaaggcctggatgagctgggtgcgccaggctcctggacagggactgcagtgggtggccagaatcaagagcacagccgatggcggcaccaccagctatgctgcccctgtggaaggccggttcatcatcagccgggacgacagccggaacatgctgtacctgcagatgaacagcctgaaaaccgaggacaccgccgtgtactactgcacctctgcccactggggccagggcacactcgtgacatctagcggaggcggaggatcaggcggcggaggaagtggcggagggggatctcagtctgtgctgacccagcctcctagcgtgtccgtgtctccaggccagaccgccaggatcacatgttctggcgaggccctgcccatgcagttcgcccactggtatcagcagaggcccggaaaggcccccgtgatcgtggtgtacaaggacagcgaaagacccagcggcgtgcccgagagattttccggctctagcagcggcacaaccgccaccctgacaatcactggcgtgcaggccgaggacgaggccgactactactgccagagccccgacagcaccaacacatacgaggtgttcggcggaggcaccaagctgaccgtgctgacaacaacccctgcccccagacctcctaccccagcccctacaattgccagccagcctctgagcctgaggcccgaggcttgtagaccagctgctggcggagccgtgcacacaagaggactggacttcgcctgcgacatctacatctgggcccctctggccggcacatgtggcgtgctgctgctgagcctcgtgatcaccctgtactgcaagtacaagcagaaacccaagtaccaagtgcggtggaagatcatcgagagctacgagggcaacagctacaccttcatcgaccccacccagctgccctacaacgagaagtgggagttcccccggaacaacctgcagttcggcaagacactgggcgctggcgcctttggcaaagtggtggaagccacagccttcggcctgggcaaagaggacgccgtgctgaaggtggccgtgaagatgctgaagtccaccgcccacgccgacgagaaagaggccctgatgagcgagctgaagatcatgagccacctgggccagcacgagaacatcgtgaatctgctgggcgcctgcacccacggcggacctgtgctcgtgattaccgagtactgctgctacggcgacctgctgaacttcctgcggagaaaggccgaggccatgctgggcccttctctgtctcccggacaggatccagagggcggcgtggactacaagaacatccacctggaaaagaaatacgtgcggcgggacagcggctttagcagccagggcgtggacacctacgtggaaatgaggcccgtgtccaccagcagcaacgacagcttcagcgagcaggacctggacaaagaagatggcagacccctggaactgcgggatctgctgcatttcagtagccaggtggcccagggcatggcctttctggccagcaagaactgcatccaccgggacgtggccgccagaaacgtgctgctgacaaacggacacgtggccaagatcggcgactttggcctggcccgggacatcatgaacgacagcaactatatcgtgaagggcaacgcccggctgcccgtgaaatggatggcccccgagagcatcttcgactgcgtgtacaccgtgcagagcgacgtgtggtcctacggcatcctgctgtgggagatcttctccctgggcctgaacccctaccctggcatcctcgtgaacagcaagttctacaagctcgtgaaggacggctaccagatggcccagcctgccttcgcccccaagaatatctacagcatcatgcaggcctgttgggccctggaacccacccacagacctaccttccagcagatctgtagctttctgcaggaacaggctcaggaagatcggagagagcgggactacaccaacctgcccagcagctctagatctggcggcagcggatctagcagctccgagctggaagaggaaagcagcagcgagcacctgacctgctgcgagcagggcgatattgcccagccactgctgcagcccaacaactaccagttctgctga

SEQ ID NO: 49 - DNA, 1188 bp, Artificial Sequence
  Synthetic Construct
atggctctgcctgtgacagctctgctgctgcctctggccctgctgctgcatgccgctagacctgaagtgcagctggtgcagtctggcggcggactggtggaacctggcggaagcctgagactgagctgtgccgtgtccggcttcgacttcgagaaggcctggatgagctgggtgcgccaggctcctggacagggactgcagtgggtggccagaatcaagagcacagccgatggcggcaccaccagctatgctgcccctgtggaaggccggttcatcatcagccgggacgacagccggaacatgctgtacctgcagatgaacagcctgaaaaccgaggacaccgccgtgtactactgcacctctgcccactggggccagggcacactcgtgacatctagcggaggcggaggatcaggcggcggaggaagtggcggagggggatctcagtctgtgctgacccagcctcctagcgtgtccgtgtctccaggccagaccgccaggatcacatgttctggcgaggccctgcccatgcagttcgcccactggtatcagcagaggcccggaaaggcccccgtgatcgtggtgtacaaggacagcgaaagacccagcggcgtgcccgagagattttccggctctagcagcggcacaaccgccaccctgacaatcactggcgtgcaggccgaggacgaggccgactactactgccagagccccgacagcaccaacacatacgaggtgttcggcggaggcaccaagctgaccgtgctgacaacaacccctgcccccagacctcctaccccagcccctacaattgccagccagcctctgagcctgaggcccgaggcttgtagaccagctgctggcggagccgtgcacacaagaggactggacttcgcctgcgacatctacctgaggcctgtgcaggctcaggcccagagcgattgcagctgctctactgtgtcccctggcgtgctggcaggcatcgtgatgggagatctggtgctgacagtgctgatcgccctggccgtgtatttcctgggcagactggtgcctagaggcaggggagctgccgaagccgccaccagaaagcagcggatcaccgagacagagagcccctaccaggaactgcagggacagcggagcgacgtgtacagcgacctgaacacccagaggccctactacaagtga

SEQ ID NO: 50 - DNA, 1785 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggaccaccacccctgcccctagaccacctacacccgccccaacaatcgccagccagcctctgtctctgaggcccgaggcttgtagaccagctgctggcggagccgtgcacacaagaggactggacttcgcctgcgacatctacatctgggcccctctggccggcacatgtggcgtgctgctgctgagcctcgtgatcaccctgtactgcaagcggggcagaaagaagctgctgtacatcttcaagcagcccttcatgcggcccgtgcagaccacccaggaagaggacggctgctcctgcagattccccgaggaagaagaaggcggctgcgagctgtctagaggcgtccaggtggaaaccatcagcccaggcgacggcagaaccttccctaagaggggccagacctgcgtggtgcactacaccggcatgctggaagatggcaagaagttcgacagcagccgggaccggaacaagcccttcaagttcatgctgggcaagcaggaagtgatccggggctgggaagagggcgtggcacagatgtctgtgggccagagagccaagctgaccatctcccccgattacgcctacggcgccacaggccaccctggcatcattcctccacacgccacactggtgttcgacgtggaactgctgaagctggaaaccaggggggtccaggtggaaacaatctctcccggcgacggccggacctttccaaaacggggacagacatgtgtggtgcattatacagggatgctggaagatgggaaaaaattcgattcctcccgcgaccgcaacaaaccttttaagtttatgctggggaaacaggaagtgattagaggctgggaagagggggtggcacagatgagcgtgggacagcgggccaaactgacaatcagccctgactatgcctatggggccaccggacaccccggaatcatcccacctcatgctaccctggtgtttgatgtggaactgctgaaactggaatga

SEQ ID NO: 51 - DNA, 771 bp, Artificial Sequence
  Synthetic Construct
atggctctgcctgtgacagctctgctgctgcctctggccctgctgctgcatgccgccagacctacaacaacccctgcccccagacctcctaccccagcccctacaattgccagccagcctctgagcctgaggcccgaggcttgtagaccagctgctggcggagccgtgcacaccagaggactggatttcgcctgcgacatctacctgcggcctgtgcaggctcaggcccagagcgattgcagctgctctaccgtgtctcctggcgtgctggccggaatcgtgatgggcgatctggtgctgaccgtgctgattgccctggccgtgtacttcctgggcagactggtgcctagaggcagaggcgctgccgaagccgccaccagaaagcagagaatcaccgagacagagagcccctaccaggaactgcagggacagcggagcgacgtgtacagcgacctgaacacccagcggccctactacaagagccggatcctgtggcacgagatgtggcatgagggcctggaagaggccagcagactgtacttcggcgagcggaacgtgaagggcatgttcgaggtgctggaacccctgcacgccatgatggaaagaggccctcagaccctgaaagagacaagcttcaaccaggcctacggccgggacctgatggaagcccaggaatggtgccggaagtacatgaagtccggcaatgtgaaggacctgctgcaggcctgggacctgtactaccacgtgttccggcggatcagcaagacctga

SEQ ID NO: 52 - DNA, 1659 bp, Artificial Sequence
  Synthetic Construct
atggctctgcctgtgacagctctgctgctgcctctggccctgctgctgcatgccgctagacctgaggtgcagctgcatcagagcggccctgagctcgtgaaacctggcgcctccgtgaagatcagctgcaagaccagcggctacaccttcaccgagtacaccatgcactgggtcaagcagagccacggcaagagcctggaatggatcggcggcgtgaacccctacaacgacaacaccagctacatccggaagctgcagggcaaagtgaccctgaccgtggacagaagcagctccaccgcctacatggaactgcggagcctgaccagcgaggacagcgccgtgtacttctgcgccagatatggcggcctgcggccctactacttccccatggatttttggggccagggcaccagcgtgaccgtgtctagcggaggcggaggatctggcggcggaggaagtggcggagggggatctcaggtggtgctgtctcagagccctgccatcctgtctgccagccctggcgagagagtgacactgacctgtagggccagcagcagcgtgaactatatgcactggtatcagcagaagcccggctccagccccaaggcctggatctacgccacaagcaatctggccagcggcgtgcccgatagattttccgcctctggcagcggcacctcctactccctgaccatcagcagagtggaagccgaggacgccgccacctactactgtcagcagtggcggaccaacccccccacatttggcgccggaacaaagctggaactgaagcggaccaccacccctgcccctagaccacctacacccgccccaacaatcgccagccagcctctgtctctgaggcccgaggcttgtagaccagctgctggcggagccgtgcacacaagaggactggacttcgcctgcgacatctacatctgggcccctctggccggcacatgtggcgtgctgctgctgagcctcgtgatcaccctgtactgcagcagaggcgtccaggtggaaaccatctctcccggcgacggcagaaccttccctaagaggggccagacctgcgtggtgcactacaccggcatgctggaagatggcaagaagttcgacagcagccgggaccggaacaagcccttcaagttcatgctgggcaagcaggaagtgatccggggctgggaagagggcgtggcacagatgtctgtgggccagagagccaagctgaccatctcccccgattacgcctacggcgccacaggccaccctggcatcattcctccacacgccacactggtgttcgacgtggaactgctgaagctggaaaccaggggggtccaggtggaaacaatcagccctggcgacggccggacctttccaaagcgcggacagacatgtgtggtgcattatacagggatgctggaagatgggaaaaaattcgattcctcccgcgaccgcaacaaaccttttaagtttatgctggggaaacaggaagtgattagaggctgggaagagggggtggcacagatgagcgtgggacagcgggccaaactgactatcagcccagactatgcctatggggccaccggacaccccggaatcatcccacctcatgctaccctggtgtttgatgtggaactgctgaaactggaatga

SEQ ID NO: 53 - DNA, 2070 bp, Artificial Sequence
  Synthetic Construct
atggccctgcctgtgacagctctgctgctgcctctggccctgctgctgcatgctgccagacctctgttcacccctgtggtggtggcctgcatgagcatcatggcactcctcttgcttttgctcctcctgctgctgtacaagtacaagcagaaacccaagtaccaagtgcggtggaagatcatcgagagctacgagggcaacagctacaccttcatcgaccccacccagctgccctacaacgagaagtgggagttcccccggaacaacctgcagttcggcaagacactgggcgctggcgcctttggcaaagtggtggaagccacagccttcggcctgggcaaagaggacgccgtgctgaaggtggccgtgaagatgctgaagtccaccgcccacgccgacgagaaagaggccctgatgagcgagctgaagatcatgagccacctgggccagcacgagaacatcgtgaatctgctgggcgcctgcacccacggcggacctgtgctcgtgatcaccgagtactgctgctacggcgacctgctgaacttcctgcggagaaaggccgaggccatgctgggcccttctctgtcccctggacaggatccagagggcggcgtggactacaagaacatccacctggaaaagaaatacgtgcggcgggacagcggcttcagcagtcagggcgtggacacctacgtggaaatgcggcctgtgtccaccagcagcaacgacagcttcagcgagcaggacctggacaaagaagatggcagacccctggaactgcgggatctgctgcactttagcagccaggtggcccagggcatggcctttctggccagcaagaactgcatccaccgggacgtggccgccagaaacgtgctgctgacaaacggacacgtggccaagatcggcgactttggcctggcccgggacatcatgaacgacagcaactatatcgtgaagggcaacgcccggctgcccgtgaaatggatggcccccgagagcatcttcgactgcgtgtacaccgtgcagagcgacgtgtggtcctacggcatcctgctgtgggagatcttctccctgggcctgaacccctaccctggcatcctcgtgaacagcaagttctacaagctcgtgaaggacggctaccagatggcccagcctgccttcgcccccaagaatatctacagcattatgcaggcctgttgggccctggaacccacccacagacctaccttccagcagatctgtagctttctgcaggaacaggctcaggaagatcggagagagcgggactacaccaacctgcccagcagctctagaagcggcggcagcggatctagcagctccgagctggaagaggaaagcagcagcgagcacctgacctgctgcgagcagggcgatattgcccagcctctgctgcagcccaacaactaccagttctgcagccggatcctgtggcacgagatgtggcatgagggcctggaagaagccagcaggctgtacttcggcgagcggaacgtgaagggaatgttcgaggtgctggaacccctgcacgccatgatggaaaggggccctcagaccctgaaagagacaagcttcaaccaggcctacggccgggacctgatggaagcccaggaatggtgccggaagtacatgaagtccggcaatgtgaaggacctgctgcaggcctgggacctgtactaccacgtgttccggcggatcagcaagaccagagtgaagttctccagaagcgccgacgcccctgcctaccagcagggacagaaccagctgtacaacgagctgaacctgggcagacgggaagagtacgacgtgctggacaagcggagaggcagggaccctgagatgggcggaaagccccagagaagaaagaacccccaggaaggcctgtataacgaactgcagaaagacaagatggccgaagcctacagcgagatcggcatgaagggcgagagaaggcggggcaagggacacgatggcctgtaccagggactgagcaccgccaccaaggatacctacgacgccctgcacatgcaggctctgccccccagataa

SEQ ID NO: 54 - DNA, 21 bp, Artificial Sequence
  Synthetic Construct
ctccactcacggcaaattcaa

SEQ ID NO: 55 - DNA, 22 bp, Artificial Sequence
  Synthetic Construct
gatgacaagcttcccattctcg

SEQ ID NO: 56 - DNA, 21 bp, Artificial Sequence
  Synthetic Construct
ccgtcagccgatttgctatct

SEQ ID NO: 57 - DNA, 22 bp, Artificial Sequence
  Synthetic Construct
acggcagagaggaggttgactt

SEQ ID NO: 58 - DNA, 22 bp, Artificial Sequence
  Synthetic Construct
acaacaaaaaagcctcgtgctg

SEQ ID NO: 59 - DNA, 22 bp, Artificial Sequence
  Synthetic Construct
ccattgaggtggagagctttca

SEQ ID NO: 60 - DNA, 22 bp, Artificial Sequence
  Synthetic Construct
ggcaaacccaaggtctacgttc

SEQ ID NO: 61 - DNA, 21 bp, Artificial Sequence
  Synthetic Construct
tacctcattggccagctgctt

SEQ ID NO: 62 - DNA, 20 bp, Artificial Sequence
  Synthetic Construct
aggacctgggttggaagtgg

SEQ ID NO: 63 - DNA, 20 bp, Artificial Sequence
  Synthetic Construct
agttggcatggtagcccttg

SEQ ID NO: 64 - DNA, 29 bp, Artificial Sequence
  Synthetic Construct
tcttcctctacacagcaagctcaccgtgg

SEQ ID NO: 65 - DNA, 24 bp, Artificial Sequence
  Synthetic Construct
gagggttcttccttctcggctttc

SEQ ID NO: 66 - DNA, 25 bp, Artificial Sequence
  Synthetic Construct
ttcactctgaagaagatgcctagcc

SEQ ID NO: 67 - DNA, 20 bp, Artificial Sequence
  Synthetic Construct
acacaactgtgttcactagc

SEQ ID NO: 68 - DNA, 20 bp, Artificial Sequence
  Synthetic Construct
gtctccttaaacctgtcttg

SEQ ID NO: 69 - DNA, 23 bp, Artificial Sequence
  Synthetic Construct
acctgactcctgaggagaagtct

SEQ ID NO: 70 - protein, 15 aa, Artificial Sequence
  Synthetic Construct
GGGGSGGGGSGGGGS